(19) United States
(12) Patent Application Publication
(10) Pub. No.: US 2010/0166707 A1
(43) Pub. Date: Jul. 1, 2010

(54) EXTRACELLULAR MATRIX/METASTASIS MODIFIER GENES FOR THE PREVENTION OR INHIBITION OF METASTASIS OR GROWTH OF TUMOR AND FOR CHARACTERIZATION OF TUMOR

(75) Inventors: Kent W. Hunter, Potomac, MD (US); Nigel Crawford, N. Bethesda, MD (US); Douglas R. Lowy, Bethesda, MD (US); Xiaolan Qian, Potomac, MD (US); Hoda Anton-Culver, Newport Beach, CA (US); Agryrios Ziogas, Irvine, CA (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignees: GOVERNMENT OF THE UNITED STATES OF AMERICA, REPRES, Rockville, MD (US); THE REGENTS OF THE UNIVERSITY OF CALIFORNIA, Oakland, CA (US)

(21) Appl. No.: 12/280,260

(22) PCT Filed: Feb. 23, 2007
(86) PCT No.: PCT/US2007/004767

§ 371 Date: Oct. 7, 2008

Related U.S. Application Data
(60) Provisional application No. 60/776,643, filed on Feb. 24, 2006.
(60) Provisional application No. 60/788,463, filed on Mar. 31, 2006.

(51) Int. Cl. A61K 31/7088; C07H 21/02; C12N 15/63; C12N 5/10; A61K 35/12; C12Q 1/68; A61P 35/00

(52) U.S. Cl. 424/93.2; 536/23.1; 435/320.1; 435/325; 514/44 R; 435/6

(57) ABSTRACT

Disclosed are methods involving the administration of an extracellular matrix (ECM)/metastasis modifier gene, e.g., Anakin, Necdin, CentaurinD3 (CentD3), Csf1r, Brd4, Pi16, and Luc7l, for the prevention or inhibition of metastasis or of tumor growth. Further disclosed are methods of characterizing a tumor or cancer in a subject comprising detecting (i) a single nucleotide polymorphism (SNP) in an Anakin gene or a Brd4 gene of the subject, (ii) an amino acid substitution in an Anakin protein in the subject, or (iii) a level of expression of an Anakin gene or a Brd4 gene in the subject. Methods of screening a compound for anti-cancer activity and use of a compound with anti-cancer activity for the preparation of a medicament to treat or prevent cancer in a subject are also disclosed. Also disclosed is a method of inhibiting Sipa-1 in a subject.

### CROSS-REFERENCE TO RELATED APPLICATIONS

This patent application claims the benefit of U.S. Provisional Patent Application No. 60/776,643, filed Feb. 24, 2006, and U.S. Provisional Patent Application No. 60/788,463, filed Mar. 31, 2006, which are each incorporated by reference.

### BACKGROUND OF THE INVENTION

The process of metastasis is of great importance to the clinical management of cancer since the majority of cancer mortality is associated with metastatic disease rather than the primary tumor (Liotta et al., Principles of molecular cell biology of cancer: Cancer metastasis (4th ed.), Cancer: Principles & Practice of Oncology, ed. S. H. V. DeVita and S. A. Rosenberg, Philadelphia, Pa.: J.B. Lippincott Co., 134-149 (1993)). In most cases, cancer patients with localized tumors have significantly better prognoses than those with disseminated tumors. Since recent evidence suggests that the first stages of metastasis can be an early event (Schmidt-Kittler et al., Proc. Natl. Acad. Sci. U.S.A., 100 (13): 7737-7742 (2003)) and that 60-70% of patients have initiated the metastatic process by the time of diagnosis, a better understanding of the factors leading to tumor dissemination is of vital importance. However, even patients that have no evidence of tumor dissemination at primary diagnosis are at risk for metastatic disease. Approximately one-third of women who are sentinel lymph node negative at the time of surgical resection of the primary breast tumor will subsequently develop clinically detectable secondary tumors (Heimann et al., Cancer Res., 60 (2): 298-304 (2000)). Even patients with small primary tumors and node negative status (T1N0) at surgery have a significant chance (15-25%) of developing distant metastases (Heimann et al., J. Clin. Oncol., 18 (3): 591-599 (2000)). The foregoing shows that there is a need for a method of characterizing a tumor or a cancer in a subject, especially in terms of the metastatic capacity of a tumor.

### BRIEF SUMMARY OF THE INVENTION

The invention provides methods of preventing or inhibiting metastasis of a cancer cell in a subject. The method comprises administering a gene, or a gene product thereof, or a combination thereof, which gene is an extracellular matrix (ECM)/metastasis modifier gene. An ECM/metastasis modifier gene is a gene for which the expression correlates with the expression of one or more ECM genes. Examples of such modifier genes may include, for instance, Anakin, Necdin (Ndn), CentD3 (Centaurin D3), Csf1r, Brd4 (Bromodomain 4), Pi16, and Luc7l. Also, an ECM/metastasis modifier gene is a gene which co-localizes with the ECM genes. Additional attributes of such ECM/metastasis genes, as well as the identification of such ECM/metastasis genes, are further described herein.

In one embodiment of the inventive method, the method comprises administering to the subject a pharmaceutical composition comprising (i) a nucleic acid comprising a nucleotide sequence encoding an Anakin protein, (ii) a vector comprising the nucleic acid, (iii) a host cell comprising the vector, (iv) an Anakin gene product, or (v) a combination thereof, in an amount that is effective to inhibit or prevent metastasis of the cancer cell in the subject. In another embodiment of the method, the method comprises administering to the subject a pharmaceutical composition comprising (i) a nucleic acid comprising a nucleotide sequence encoding a Necdin protein, (ii) a vector comprising the nucleic acid, (iii) a host cell comprising the vector, (iv) a Necdin gene product, or (v) a combination thereof. In yet another embodiment of the method, the method comprises administering to the subject a pharmaceutical composition comprising (i) a nucleic acid comprising a nucleotide sequence encoding a Brd4 protein, (ii) a vector comprising the nucleic acid, (iii) a host cell comprising the vector, (iv) a Brd4 gene product, or (v) a combination thereof.

The invention also provides methods of preventing or inhibiting tumor growth in a subject. The method comprises administering an ECM/metastasis modifier gene, a gene product thereof, or a combination thereof. In one embodiment of the method, the method comprises administering to the subject a pharmaceutical composition comprising (i) a nucleic acid comprising a nucleotide sequence encoding an Anakin protein, (ii) a vector comprising the nucleic acid, (iii) a host cell comprising the vector, (iv) an Anakin gene product, or (v) a combination thereof. In another embodiment of the method, the method comprises administering to the subject a pharmaceutical composition comprising (i) a nucleic acid comprising a nucleotide sequence encoding a Necdin protein, (ii) a vector comprising the nucleic acid, (iii) a host cell comprising the vector, (iv) a Necdin gene product, or (v) a combination thereof. In another embodiment of the method, the method comprises administering to the subject a pharmaceutical composition comprising (i) a nucleic acid comprising a nucleotide sequence encoding a Brd4 protein, (ii) a vector comprising the nucleic acid, (iii) a host cell comprising the vector, (iv) a Brd4 gene product, or (v) a combination thereof. In yet another embodiment of the method, the method comprises administering to the subject a pharmaceutical composition comprising (i) a nucleic acid comprising a nucleotide sequence encoding a protein (ii) a vector comprising the nucleic acid, (iii) a host cell comprising the vector, (iv) a gene product, or (v) a combination thereof, wherein the protein or the gene product is encoded by a gene selected from the group consisting of CentD3, Csf1r, Pi16, and Luc7l.

Isolated, purified, or synthetic nucleic acids, inclusive of diagnostic primers and probes, are further provided herein for use in the inventive methods. The invention further provides isolated, purified, or synthetic antibodies, or antigen binding portions thereof, which specifically bind to a murine Anakin protein or an Anakin allelic variant. Kits comprising diagnostic agents and pharmaceutical compositions comprising therapeutic agents are also provided by the invention. In one pharmaceutical composition, the composition comprises (i) a nucleic acid comprising a nucleotide sequence encoding a protein, (ii) a vector comprising the nucleic acid, (iii) a host cell comprising the vector, (iv) a gene product, or (v) a combination thereof, wherein the protein or gene product is encoded by a gene selected from the group consisting of Anakin, Ndn, CentD3, Csf1r, Brd4, Pi16, and Luc7l, and a pharmaceutically acceptable carrier.

In addition, methods of characterizing a tumor or a cancer in a subject are provided herein. In one method, the method comprises detecting (i) a SNP in an Anakin gene of the subject, (ii) an amino acid substitution in an Anakin protein in the subject, or (iii) a level of expression of an Anakin gene in the subject. In another method, the method comprises detecting (i) a SNP in a Brd4 gene of the subject or (ii) a level of expression of a Brd4 gene in the subject.

Further provided by the invention is a method for screening a compound for anti-cancer activity. The method comprises (a) providing a cell that (i) under-expresses a nucleic acid comprising a nucleotide sequence encoding an Anakin protein or a Brd4 protein or (ii) comprises an Anakin or Brd4 allelic variant, (b) contacting the cell with a compound of interest, and (c) assaying for anti-cancer activity.

The invention also provides use of a compound with anti-cancer activity for the preparation of a medicament to treat or prevent cancer in a subject who has been tested for (i) a SNP in an Anakin gene or a Brd4 gene of the subject, (ii) an amino acid substitution in an Anakin protein in the subject, or (iii) an expression level of an Anakin gene or Brd4 gene in the subject.

The invention further provides a method of inhibiting Sipa-1 in a subject in need thereof. The method comprises administering to the subject (i) a nucleic acid comprising a nucleotide sequence encoding an Anakin protein, (ii) a vector comprising the nucleic acid, (iii) a host cell comprising the vector, (iv) an Anakin gene product, or (v) a combination thereof.

### BRIEF DESCRIPTION OF THE DRAWINGS

FIGS. 1A-1D depict a series of Western blots of cells co-transfected with empty vector or vector encoding Sipa-1 and with empty vector or vector encoding Anakin or AQP2. FIG. 1A is a Western blot of the co-transfected cells immunoprecipitated for Sipa-1, V5, or AQP2 and immunoblotted with anti-V5 antibody. FIG. 1B is a Western blot of the co-transfected cells immunoprecipitated for Sipa-1, V5, or AQP2 and immunoblotted with anti-AQP2 antibody. FIG. 1C is a Western blot of the cell extracts of co-transfected cells immunoblotted with anti-V5 antibody. FIG. 1D is a Western blot of the cell extracts of co-transfected cells immunoblotted with anti-AQP2 antibody.

FIGS. 2A-2C depict a series of Western blots of cells co-transfected with empty vector or vector encoding Sipa-1, with empty vector or vector encoding Anakin or AQP2, and with vector encoding Epac-HA, a guanine nucleotide exchange factor for Rap. FIG. 2A is a Western blot of the cell fraction of the cell extracts of co-transfected cells, which cell fraction was pulled down with RalGDS beads, and immunoblotted with anti-Rap-1 antibody. FIG. 2B is a Western blot of the cell extracts of the co-transfected cells immunoblotted with an anti-Rap-1 antibody. FIG. 2C is Western blot of the cell extracts of the co-transfected cells immunoblotted with an anti-Epac HA antibody.

FIG. 3 depicts a Western blot of Mvt1 cells stably transfected with vector encoding Anakin (clone 1 and clone 2), of Mvt1 cells stably transfected with vector encoding β-galactosidase (β-gal clone 3), or untransfected Mvt1 cells immunoblotted with anti-Kai1 antibody.

FIG. 4 depicts a graph of the weight (in grams) of tumors of mice subcutaneously implanted with Mvt1 cells stably transfected with vector encoding Anakin (Anakin 1-Anakin 4) or of mice implanted with an equal number of Mvt1 cells transfected with vector encoding β-galactosidase.

FIG. 5 depicts a graph of the relative (β-galactosidase (β-gal) activity of cells transfected with a β-gal reporter construct comprising the promoter of the Anakin gene from either an AKR tumor (high metastatic capacity; white bar) or a DBA tumor (low metastatic capacity; diagonal-lined bar).

FIG. 6 depicts the average tumor weight (in grams) obtained from mice implanted with Mvt-1 cells expressing a control β-gal gene (β-gal Clonal Isolate 1 (diagonal lined bar) and β-gal Clonal Isolate 2 (criss-crossed bar)) or Brd4 (Brd4 Clonal Isolate 1 (vertical lined bar), Brd4 Clonal Isolate 2 (dashed lined bar), Brd4 Clonal Isolate 3 (plus signed bar), and Brd4 Clonal Isolate 4 (bar with open triangles)).

FIG. 7 depicts the pulmonary metastasis count of mice implanted with Mvt-1 cells expressing a control β-gal gene (β-gal Clonal Isolate 1 (diagonal lined bar) and β-gal Clonal Isolate 2 (criss-crossed bar)) or Brd4 (Brd4 Clonal Isolate 1 (vertical lined bar), Brd4 Clonal Isolate 2 (dashed lined bar), Brd4 Clonal Isolate 3 (plus signed bar), and Brd4 Clonal Isolate 4 (bar with open triangles)).

### DETAILED DESCRIPTION OF THE INVENTION

The invention provides methods of preventing or inhibiting metastasis of a cancer cell in a subject and methods of preventing or inhibiting tumor growth in a subject, which methods involve the administration of an ECM/metastasis modifier gene, or a gene product thereof. The invention also provides methods of characterizing a tumor or a cancer in a subject comprising detecting (i) a single nucleotide polymorphism (SNP) in an ECM/metastasis modifier gene in the subject, (ii) an amino acid substitution in a protein encoded by such a gene of the subject, or (iii) an expression level of such a gene in the subject.

As used herein, the term “ECM/metastasis modifier gene” refers to a gene that has expression levels that correlate with the expression levels of ECM genes. Desirably, the ECM/metastasis modifier gene additionally (1) maps to an ECM efficiency quantitative trait loci (eQTL) interval, (2) contains polymorphisms in the coding or promoter region of the gene, (3) alters the endogenous ECM gene transcription upon in vitro ectopic expression of the ECM/metastasis modifier gene, (4) alters metastasis in transplant assays upon in vitro ectopic expression of the ECM/metastasis modifier gene, and/or (5) is associated with metastatic breast cancer in human epidemiological studies. The evidence provided herein suggests that Anakin, Ndn, CentD3, Csf 1r, Brd4, Pi16, and Luc7l are ECM/metastasis modifier genes.

With respect to the inventive methods, the phrase “metastasis of a cancer cell” refers to the transmission of a cancer cell from an original site to one or more sites elsewhere in the body, e.g., from one organ or part to another not directly connected with it by way of, for example, blood vessels or lymphatics. The metastasis of a cancer cell can, for example, lead to the formation of a secondary or subsequent tumor at a site other than the location of the primary tumor. The cancer cell of the inventive methods can be a cell of any cancer, such as those cancers described herein. Preferably, the cancer cell is a metastatic cancer cell.

In one embodiment of the inventive method of preventing or inhibiting metastasis of a cancer cell, the method comprises administering to the subject a pharmaceutical composition comprising (i) a nucleic acid comprising a nucleotide sequence encoding an Anakin protein, (ii) a vector comprising the nucleic acid, (iii) a host cell comprising the vector, (iv) an Anakin gene product, or (v) a combination thereof, in an amount that is effective to inhibit or prevent metastasis of the cancer cell in the subject.

In this regard, the invention further provides a pharmaceutical composition comprising (i) a nucleic acid comprising a nucleotide sequence encoding an Anakin protein, (ii) a vector comprising the nucleic acid, (iii) a host cell comprising the vector, (iv) an Anakin gene product, or (v) a combination thereof, and a pharmaceutically acceptable carrier.

Anakin proteins, as well as nucleic acids comprising nucleotide sequences each encoding an Anakin protein, are known in the art. For instance, the amino acid sequence of the human Anakin protein is available from the GenBank database of the National Center for Biotechnology Information (NCBI) website as Accession No. NP_{—}0055871 and herein as SEQ ID NO: 1. Also, a nucleotide sequence encoding the human Anakin protein is available from the GenBank database as Accession No. NM_{—}015056 and herein as SEQ ID NO: 2. Further, the amino acid sequence of the murine Anakin protein is available from the GenBank database of the NCBI website as Accession No. NP_{—}082520.1 and herein as SEQ ID NO: 3. Also, a nucleotide sequence encoding the murine Anakin protein is available from the GenBank database as Accession No. NM_{—}028244 and herein as SEQ ID NO: 4.

In another embodiment of the inventive method of preventing or inhibiting metastasis of a cancer cell, the method comprises administering to the subject a pharmaceutical composition comprising (i) a nucleic acid comprising a nucleotide sequence encoding a Necdin protein, (ii) a vector comprising the nucleic acid, (iii) a host cell comprising the vector, (iv) a Necdin gene product, or (v) a combination thereof, and a pharmaceutically acceptable carrier in an amount that is effective to inhibit or prevent metastasis of the cancer cell in the subject.

In this regard, the invention further provides a pharmaceutical composition comprising (i) a nucleic acid comprising a nucleotide sequence encoding a Necdin protein, (ii) a vector comprising the nucleic acid, (iii) a host cell comprising the vector, (iv) a Necdin gene product, or (v) a combination thereof, and a pharmaceutically acceptable carrier.

Necdin proteins, as well as nucleic acids comprising nucleotide sequences each encoding a Necdin protein, are known in the art. For instance, the amino acid sequence of the human Necdin protein is available from the GenBank database of the NCBI website as Accession No. NP_{—}002478 and herein as SEQ ID NO: 9. Also, a nucleotide sequence encoding the human Necdin protein is available from the GenBank database as Accession No. NM_{—}002487 and herein as SEQ ID NO: 10. The amino acid sequence of the mouse Necdin protein is available from the GenBank database of the NCBI website as Accession No. NP_{—}035012 and herein as SEQ ID NO: 11. Also, a nucleotide sequence encoding the human Necdin protein is available from the GenBank database as Accession No. NM_{—}010882 and herein as SEQ ID NO: 12.

In another embodiment of the inventive method of preventing or inhibiting metastasis of a cancer cell, the method comprises administering to the subject a pharmaceutical composition comprising (i) a nucleic acid comprising a nucleotide sequence encoding a Brd4 protein, (ii) a vector comprising the nucleic acid, (iii) a host cell comprising the vector, (iv) a Brd4 gene product, or (v) a combination thereof, in an amount that is effective to inhibit or prevent metastasis of the cancer cell in the subject.

In this regard, the invention further provides a pharmaceutical composition comprising (i) a nucleic acid comprising a nucleotide sequence encoding a Brd4 protein, (ii) a vector comprising the nucleic acid, (iii) a host cell comprising the vector, (iv) a Brd4 gene product, or (v) a combination thereof, and a pharmaceutically acceptable carrier.

Brd4 proteins, as well as nucleic•acids comprising nucleotide sequences each encoding a Brd4 protein, are known in the art. For instance, the amino acid sequence of the long isoform of the human Brd4 protein is available from the GenBank database of the National Center for Biotechnology Information (NCBI) website as Accession No. NP_{—}490597.1 and herein as SEQ ID NO: 109. Also, a nucleotide sequence encoding the long isoform of the human Brd4 protein is available from the GenBank database as Accession No. NM_{—}058243.1 and herein as SEQ ID NO: 108. The amino acid sequence of the short isoform of the human Brd4 protein is available from the GenBank database of the National Center for Biotechnology Information (NCBI) website as Accession No. NP_{—}055114 and herein as SEQ ID NO: 111. Also, a nucleotide sequence encoding the short isoform of the human Brd4 protein is available from the GenBank database as Accession No. NM_{—}014299.1 and herein as SEQ ID NO: 110. Further, the amino acid sequence of one isoform of the murine Brd4 protein is available from the GenBank database of the NCBI website as Accession No. NP_{—}065254.2. The nucleotide sequence encoding this isoform is available from the GenBank database as Accession No. NM_{—}020508.2. The amino acid sequence of another isoform of the murine Brd4 protein is available from the GenBank database of the NCBI website as Accession No. NP_{—}932762.1 and its corresponding nucleotide sequence is available as Accession No. NM_{—}198094.1.

For purposes herein “gene product” refers to any molecule encoded by a gene. Gene products include, for example, proteins, mRNAs, primary RNA transcripts, alternatively spliced transcripts, allelic variants, and the like. Thus, an “Anakin gene product” as used herein refers to a molecule encoded by an Anakin gene and can be, for instance, an Anakin protein or an Anakin mRNA. Likewise, a “Necdin gene product” as used herein refers to a molecule encoded by a Necdin gene and can be, for instance, a Necdin protein or a Necdin mRNA.

With respect to the inventive methods and materials described herein, the term “protein” is meant a molecule comprising one or more (e.g., one, two, three, four, five, or more) polypeptide chains. The protein can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The protein can be glycosylated, amidated, carboxylated, phosphorylated, esterified, N-acylated, cyclized via, e.g., a disulfide bridge, or converted into an acid addition salt and/or optionally dimerized or polymerized, or conjugated.

When the protein is in the form of a salt, preferably, the protein is in the form of a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

For purposes herein, the term “protein” encompasses functional portions and functional variants of the parent protein. For instance, Anakin proteins encompass functional portions and functional variants of an Anaking protein, e.g., SEQ ID NO: 1 or 3. Also, for instance, Necdin proteins encompass functional portions and functional variants of a Necdin protein, e.g., the Necdin protein comprising the amino acid sequence of SEQ ID NO: 9. Further, for example, Brd4 proteins encompass functional portions and functional variants of Brd4 proteins, e.g., SEQ ID NO: 109 or 111.

The term “functional portion” when used in reference to a protein refers to any part or fragment of the protein, which part or fragment retains the biological activity of the protein of which it is a part. Functional portions encompass, for example, those parts of a protein (the parent protein) that retain the ability to function to a similar extent, the same extent, or to a higher extent, as the parent protein. For example, a functional portion of an Anakin protein (e.g., a protein comprising the amino acid sequence of SEQ ID NO: 1 or 3) retains the ability to prevent or inhibit metastasis to a similar extent, the same extent, or to a higher extent, as the parent Anakin protein. Also, for example, a functional portion of a Necdin protein (e.g., a protein comprising the amino acid sequence of SEQ ID NO: 9) retains the ability to prevent or inhibit metastasis to a similar extent, the same extent, or to a higher extent, as the parent Necdin protein. Furthermore, for example, a functional portion of a Brd4 protein (e.g., a protein comprising the amino acid sequence of SEQ ID NO: 109 or 111) retains the ability to prevent or inhibit metastasis to a similar extent, the same extent, or to a higher extent, as the parent Brd4 protein. In reference to the parent protein, the functional portion can comprise, for instance, about 10%, 25%, 30%, 50%, 68%, 80%, 90%, 95%, or more of the parent protein. The functional portion can comprise additional amino acids at the amino or carboxy terminus of the portion, or at both termini, which additional amino acids are not found in the amino acid sequence of the parent protein. Desirably, the additional amino acids do not interfere with the biological function of the functional portion

The term “functional variant” as used herein refers to a protein having substantial or significant sequence identity or similarity to a parent protein, which functional variant retains the biological activity of the protein of which it is a variant. Functional variants encompass, for example, those variants of a protein (the parent protein) that retain the ability to bind to function to a similar extent, the same extent, or to a higher extent, as the parent protein. For instance, a functional variant of an Anakin protein (e.g., a protein comprising the amino acid sequence of SEQ ID NO: 1 or 3) retains the ability to prevent or inhibit metastasis to a similar extent, the same extent, or to a higher extent, as the parent Anakin protein. Also, for instance, a functional variant of a Necdin protein (e.g., a protein comprising the amino acid sequence of SEQ ID NO: 9) retains the ability to prevent or inhibit metastasis to a similar extent, the same extent, or to a higher extent, as the parent Necdin protein. Furthermore, for instance, a functional variant of a Brd4 protein (e.g., a protein comprising the amino acid sequence of SEQ ID NO: 109 or 111) retains the ability to prevent or inhibit metastasis to a similar extent, the same extent, or to a higher extent, as the parent Brd4 protein. In reference to the parent protein, the functional variant can, for instance, be at least about 30%, 50%, 75%, 80%, 90%, 98% or more identical to the parent protein.

The functional variant can, for example, comprise the amino acid sequence of the parent protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the protein.

The proteins of the inventive pharmaceutical compositions (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the proteins of the inventive pharmaceutical compositions (including functional portions and functional variants thereof) can be isolated and/or purified from a source, such as a plant, a bacterium, an insect, a mammal, e.g., a rat, a human, etc. Methods of isolation and purification are well-known in the art. Alternatively, the proteins of the inventive pharmaceutical compositions (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the proteins of the inventive pharmaceutical compositions (including functional portions and functional variants thereof) can be synthetic, recombinant, isolated, and/or purified.

The invention further provides methods of preventing or inhibiting tumor growth in a subject. In one embodiment, the method comprises administering to the subject a pharmaceutical composition comprising (i) a nucleic acid comprising a nucleotide sequence encoding an Anakin protein, (ii) a vector comprising the nucleic acid, (iii) a host cell comprising the vector, (iv) an Anakin gene product, or (v) a combination thereof, and a pharmaceutically acceptable carrier.

In another embodiment of the inventive method of preventing or inhibiting tumor growth in a subject, the method comprises administering to the subject a pharmaceutical composition comprising (i) a nucleic acid comprising a nucleotide sequence encoding a Necdin protein, (ii) a vector comprising the nucleic acid, (iii) a host cell comprising the vector, (iv) a Necdin gene product, or (v) a combination thereof, and a pharmaceutically acceptable carrier.

In another embodiment of the inventive method of preventing or inhibiting tumor growth in a subject, the method comprises administering to the subject a pharmaceutical composition comprising (i) a nucleic acid comprising a nucleotide sequence encoding a Brd4 protein, (ii) a vector comprising the nucleic acid, (iii) a host cell comprising the vector, (iv) a Brd4 gene product, or (v) a combination thereof, and a pharmaceutically acceptable carrier.

In yet another embodiment of the inventive method of preventing or inhibiting tumor growth in a subject, the method comprises administering to the subject a pharmaceutical composition comprising (i) a nucleic acid comprising a nucleotide sequence encoding a protein, (ii) a vector comprising the nucleic acid, (iii) a host cell comprising the vector, (iv) a gene product, or (v) a combination thereof, wherein the protein or gene product is encoded by a gene selected from the group consisting of CentaurinD3 (CentD3), Csf1r, Pi16, and Luc7l, and a pharmaceutically acceptable carrier.

In this regard, the invention further provides a pharmaceutical composition comprising (i) a nucleic acid comprising a nucleotide sequence encoding a protein, (ii) a vector comprising the nucleic acid, (iii) a host cell comprising the vector, (iv) a gene product, or (v) a combination thereof, wherein the protein or gene product is encoded by a gene selected from the group consisting of CentD3, Csf1r, Pi16, and Luc7l, and a pharmaceutically acceptable carrier.

CentD3, Csf1r, Brd4, Pi16, and Luc7l genes are known in the art, and include the genes comprising the nucleotide sequences of Gene Entrez Nos. 106592 (CentD3), 12978 (Csf1r), 57261 (Brd4), 74116 (Pi16), and 66978 (Luc7l) and herein as SEQ ID NOs: 14, 16, 18, 20, and 22, respectively. Additional genes include SEQ ID NOs: 24 (Brd4) and 26 (Luc7l).

The Anakin protein of the inventive pharmaceutical composition encompasses functional portions and functional variants of an Anakin protein, e.g., the Anakin protein comprising the amino acid sequence of SEQ ID NO: 1 or 3. Similarly, the Necdin protein of the inventive pharmaceutical composition encompasses functional portions and functional variants of a Necdin protein, e.g., the Necdin protein comprising the amino acid sequence of SEQ ID NO: 9. Also, the Brd4 protein of the inventive pharmaceutical composition encompasses functional portions and functional variants of a Brd4 protein, e.g., the Brd4 protein comprising the amino acid sequence of SEQ ID NO: 109 or 111. Likewise, the protein encoded by a gene selected from the group consisting of CentaurinD3 (CentD3), Csf1r, Pi16, and Luc7l, encompasses functional portions and functional variants of the corresponding parent protein encoded by the gene.

In an embodiment of the inventive methods of preventing or inhibiting metastasis of a cancer cell in a subject, the subject is a mammal that is afflicted with cancer and the method effectively treats cancer. In another embodiment of the inventive method of preventing or inhibiting metastasis of a cancer cell in a subject, the subject is a mammal that has a predisposition to cancer and the method effectively prevents cancer.

Likewise, in an embodiment of the inventive methods of preventing or inhibiting tumor growth in a subject, the subject is a mammal that is afflicted with cancer and the method effectively treats cancer. In another embodiment of the inventive method of preventing or inhibiting tumor growth in a subject, the subject is a mammal that has a predisposition to cancer and the method effectively prevents cancer.

In these respects, the invention further provides methods of preventing or treating cancer in a subject. In particular, the invention provides a method of preventing or treating cancer in a subject comprising administering to the subject a pharmaceutical composition comprising (i) a nucleic acid comprising a nucleotide sequence encoding an Anakin protein, a Necdin protein, or a Brd4 protein (ii) a vector comprising the nucleic acid, (iii) a host cell comprising the vector, (iv) an Anakin gene product, a Necdin gene product, or a Brd4 gene product, or (v) a combination thereof, and a pharmaceutically acceptable carrier.

As would be appreciated by one ordinarily skilled, the inventive pharmaceutical compositions can be administered in any suitable form. For example, when the pharmaceutical composition comprises a nucleic acid, the nucleic acid can be administered in the form of a liposome. Alternatively, the nucleic acid can be administered in the form of a vector.

The vector of the inventive pharmaceutical compositions can be any suitable vector, and can be used to transform or transfect any suitable host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the PET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech).

The vectors of the inventive pharmaceutical compositions can be prepared using standard recombinant DNA techniques described in, for example, Sambrook et al., supra, and Ausubel et al., supra. Constructs of vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

Desirably, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the vectors of the inventive pharmaceutical compositions include, for instance, neomycin/G418 resistance genes, hygromycin resistance genes, histidinol resistance genes, tetracycline resistance genes, and ampicillin resistance genes.

The vector can comprise a native or normative promoter operably linked to the siRNA or shRNA of the invention. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The vectors of the inventive pharmaceutical compositions can be designed for either transient expression, for stable expression, or for both. Also, the vectors can be made for constitutive expression or for inducible expression. Further, the vectors can be made to include a suicide gene.

As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

Alternatively, the nucleic acid can be administered upon administration of a host cell comprising any of the vectors described herein. The term “host cell” as used herein refers to any type of cell that can contain the vector of the inventive pharmaceutical composition. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the vector, the host cell is preferably a prokaryotic cell, e.g., a DH5α cell.

One of ordinary skill in the art will readily appreciate that the nucleic acids, vectors, host cells, and gene products of the inventive pharmaceutical compositions (herein collectively referred to as “therapeutic or diagnostic agents”) can be modified in any number of ways, such that the therapeutic efficacy of the therapeutic or diagnostic agent is increased through the modification. For instance, the therapeutic or diagnostic agents can be conjugated either directly or indirectly through a linker to a targeting moiety. The practice of conjugating compounds or therapeutic or diagnostic agents to targeting moieties is known in the art. See, for instance, Wadwa et al., J. Drug Targeting 3: 111 (1995) and U.S. Pat. No. 5,087,616. The term “targeting moiety” as used herein, refers to any molecule or agent that specifically recognizes and binds to a cell-surface receptor, such that the targeting moiety directs the delivery of the therapeutic or diagnostic agent to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). The term “linker” as used herein, refers to any agent or molecule that bridges the therapeutic or diagnostic agent to the targeting moiety. One of ordinary skill in the art recognizes that sites on the therapeutic or diagnostic agent which are not necessary for the function of the therapeutic or diagnostic agent are ideal sites for attaching a linker and/or a targeting moiety, provided that the linker and/or targeting moiety, once attached to the therapeutic or diagnostic agent do(es) not interfere with the function of the therapeutic or diagnostic agent, i.e., the ability to inhibit or prevent metastasis of a cancer cell, the ability to prevent or inhibit tumor growth, or the ability to treat or prevent cancer.

Alternatively, the therapeutic or diagnostic agent can be modified into a depot form, such that the manner in which the therapeutic or diagnostic agent is released into the body to which it is administered is controlled with respect to time and location within the body (see, for example, U.S. Pat. No. 4,450,150). Depot forms of therapeutic or diagnostic agent can be, for example, an implantable composition comprising the therapeutic or diagnostic agent and a porous or non-porous material, such as a polymer, wherein the therapeutic or diagnostic agent is encapsulated by or diffused throughout the material and/or degradation of the non-porous material. The depot is then implanted into the desired location within the body and the therapeutic or diagnostic agent is released from the implant at a predetermined rate.

With respect to the inventive pharmaceutical compositions, the pharmaceutically acceptable carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular therapeutic or diagnostic agent, as well as by the particular method used to administer the therapeutic or diagnostic agent. Accordingly, there are a variety of suitable formulations of the pharmaceutical composition of the invention. The following formulations for oral, aerosol, parenteral, subcutaneous, intravenous, intramuscular, intraarterial, intrathecal, interperitoneal, rectal, and vaginal administration are exemplary and are in no way limiting. More than one route can be used to administer the therapeutic or diagnostic agent and in instances, a particular route can provide a more immediate and more effective response than another route.

It will be appreciated by one of skill in the art that, in addition to the following described pharmaceutical compositions, the therapeutic or diagnostic agents can be formulated as inclusion complexes, such as cyclodextrin inclusion complexes, or liposomes.

Topical formulations are well-known to those of skill in the art. Such formulations are particularly suitable in the context of the present invention for application to the skin.

Formulations suitable for oral administration can consist of (a) liquid solutions, such as an effective amount of the therapeutic or diagnostic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic or diagnostic agent in a flavor, usually sucrose and acacia or tragacanth, as well as pastilles comprising the therapeutic or diagnostic agent in an inert base, such as gelatin and glycerin, or sucrose and acacia, emulsions, gels, and the like containing, in addition to, such excipients as are known in the art.

The therapeutic or diagnostic agent, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The therapeutic or diagnostic agent can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the therapeutic or diagnostic agent in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the ldnd previously described.

Injectable formulations are in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHD Handbook on Injectable Drugs, Toissel, 4th ed., pages 622-630 (1986)).

Additionally, the therapeutic or diagnostic agent, or compositions comprising therapeutic or diagnostic agent, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

For purposes of all of the inventive methods, the administered amount or dose of the therapeutic or diagnostic agent should be sufficient to effect a therapeutic response in the subject or animal over a reasonable time frame. For example, the dose of the therapeutic or diagnostic agent should be sufficient to prevent or inhibit metastasis in a period of from about 2 hours or longer, e.g., 12 to 24 or more hours, from the time of administration. Also, for instance, the dose of the therapeutic or diagnostic agent should be sufficient to prevent or inhibit tumor growth in a period of from about 2 hours of longer, e.g., 12 to 24 or more hours, from the time of administration. In certain embodiments, the time period could be even longer. The dose will be determined by the efficacy of the particular therapeutic or diagnostic agent and the condition of the animal (e.g., human), as well as the body weight of the animal (e.g., human) to be treated. Many assays for determining an administered dose are known in the art. For purposes of the invention, an assay, which comprises comparing the extent to which the metastasis of a cancer cell is inhibited upon administration of a given dose of a therapeutic or diagnostic agent to a mammal among a set of mammals of which is each given a different dose of the therapeutic or diagnostic agent could be used to determine a starting dose to be administered to a mammal. The extent to which the metastasis of a cancer cell is inhibited or to which the tumor growth is inhibited upon administration of a certain dose can be assayed by methods known in the art, including, for instance, the method described herein as Examples 5, 6, and 8.

The dose of the therapeutic or diagnostic agent also will be determined by the existence, nature and extent of any adverse side effects that might accompany the administration of a particular therapeutic or diagnostic agent. Typically, the attending physician will decide the dosage of the therapeutic or diagnostic agent with which to treat each individual patient, taking into consideration a variety of factors, such as age, body weight, general health, diet, sex, therapeutic or diagnostic agent to be administered, route of administration, and the severity of the condition being treated. By way of example and not intending to limit the present invention, the dose of the therapeutic or diagnostic agent can be about 0.001 to about 1000 mg/kg body weight of the subject being treated/day, from about 0.01 to about 10 mg/kg body weight/day, about 0.01 mg to about 1 mg/kg body weight/day.

The invention also provides methods of detecting cancer or a predisposition to cancer in a subject. In one method, the method comprises detecting (i) a single nucleotide polymorphism (SNP) in an Anakin gene of the subject, (ii) an amino acid substitution in an Anakin protein in the subject, or (iii) an expression level of an Anakin gene in the subject, wherein detection of (i) or (ii) or an under-expression of the Anakin gene is indicative of cancer or a predisposition to cancer in the subject. In another method, the method comprises detecting (i) a single nucleotide polymorphism (SNP) in a Brd4 gene of the subject or (ii) an expression level of a Brd4 gene in the subject, wherein detection of (i) or an under-expression of the Brd4 gene is indicative of cancer or a predisposition to cancer in the subject.

The data presented herein supports that SNPs of an Anakin gene or a Brd4 gene, expression levels of an Anakin gene or a Brd4 gene, and amino acid substitutions of an Anakin protein, are further useful in methods other than diagnostic methods. For example, the data presented herein as Example 7 demonstrates that a SNP in an Anakin gene correlates with certain characteristics of tumors and cancers. Also, for example, the data presented herein as Example 9 demonstrates that a SNP in a Brd4 gene correlates with, certain characteristics of tumors and cancers. Furthermore, the data presented herein demonstrates that low expression or an under-expression of an Anakin gene or a Brd4 gene is associated with highly metastatic tumors. In this regard, the invention provides methods of characterizing a tumor or a cancer in a subject. In one method, the method comprises detecting (i) a single nucleotide polymorphism (SNP) in an Anakin gene of the subject, (ii) an amino acid substitution in an Anakin protein in the subject, or (iii) an expression level of an Anakin gene in the subject. In another method, the method comprises detecting (i) a single nucleotide polymorphism (SNP) in a Brd4 gene of the subject or (ii) an expression level of a Brd4 gene in the subject.

The inventive method of characterizing a tumor or cancer can include characterizing one, two, or any number of tumor or cancer characteristics. Preferably, the method characterizes the tumor or cancer in terms of one or more of metastatic capacity, tumor stage, tumor grade, nodal involvement, regional metastasis, distant metastasis, tumor size, and/or sex hormone receptor status.

The term “metastatic capacity” as used herein is synonymous with the term “metastatic potential” and refers to the chance that a tumor will become metastatic. The metastatic capacity of a tumor can range from high to low, e.g., from 100% to 0%. In this respect, the metastatic capacity of a tumor can be, for instance, 100%, 90%, 80%, 75%, 60%, 50%, 40%, 30%, 25%, 15%, 10%, 5%, 3%, 1%, or 0%. For example, a tumor having a metastatic capacity of 100% is a tumor having a 100% chance of becoming metastatic. Also, a tumor having a metastatic capacity of 50%, for example, is a tumor having a 50% chance of becoming metastatic. Further, a tumor with a metastatic capacity of 25%, for instance, is a tumor having a 25% chance of becoming metastatic.

“Tumor stage” as used herein refers to whether the cells of the tumor or cancer have remained localized (e.g., cells of the tumor or cancer have not metastasized from the primary tumor), have metastasized to only regional or surrounding tissues relative to the site of the primary tumor, or have metastasized to tissues that are distant from the site of the primary tumor.

“Tumor grade” as used herein refers to the degree of abnormality of cancer cells, a measure of differentiation, and/or the extent to which cancer cells are similar in appearance and function to healthy cells of the same tissue type. The degree of differentiation often relates to the clinical behavior of the particular tumor. Based on the microscopic appearance of cancer cells, pathologists commonly describe tumor grade by degrees of severity. Such terms are standard pathology terms, and are known and understood by one of ordinary skill in the art (see Crawford et al., Breast Cancer Research 8:R16; e-publication on Mar. 21, 2006)).

“Nodal involvement” as used herein refers to the presence of a tumor cell within a lymph node as detected by, for example, microscopic examination of a section of a lymph node.

“Regional metastasis” as used herein means the metastasis of a tumor cell to a region that is relatively close to the origin, i.e., the site of the primary tumor. For example, regional metastasis includes metastasis of a tumor cell to a regional lymph node that drains the primary tumor, i.e., that is connected to the primary tumor by way of the lymphatic system. Also, regional metastasis can be, for instance, the metastasis of a tumor cell to the liver in the case of a primary tumor that is in contact with the portal circulation. Further, regional metastasis can be, for example, metastasis to a mesenteric lymph node in the case of colon cancer. Furthermore, regional metastasis can be, for instance, metastasis to an axillary lymph node in the case of breast cancer.

The term “distant metastasis” as used herein refers to metastasis of a tumor cell to a region that is non-contiguous with the primary tumor (e.g., not connected to the primary tumor by way of the lymphatic or circulatory system). For instance, distant metastasis can be metastasis of a tumor cell to the brain in the case of breast cancer, a lung in the case of colon cancer, and an adrenal gland in the case of lung cancer.

“Sex hormone receptor status” as used herein means the status of whether a sex hormone receptor is expressed in the tumor cells or cancer cells. Sex hormone receptors are known in the art, including, for instance, the estrogen receptor, the testosterone receptor, and the progesterone receptor. Preferably, when characterizing certain cancers, such as breast cancer, the sex hormone receptor is the estrogen receptor or progesterone receptor.

As the metastatic capacity, tumor stage, tumor grade, nodal involvement, regional metastasis, distant metastasis, tumor size, and sex hormone receptor status are factors when considering a stage of a cancer, e.g., breast cancer, the inventive method of characterizing a tumor or cancer in a subject preferably effectively stages the tumor or cancer.

Further, as, for instance, the metastatic capacity, tumor stage, tumor grade, nodal involvement, regional metastasis, distant metastasis, tumor size, and sex hormone receptor status are factors considered when determining a treatment for a subject afflicted with a tumor or cancer, the invention further provides methods of determining a treatment for a subject afflicted with a tumor or a cancer. In one method, the method comprises detecting (i) a single nucleotide polymorphism (SNP) in an Anakin gene of the subject, (ii) an amino acid substitution in an Anakin protein in the subject, or (iii) an expression level of an Anakin gene in the subject. In another method, the method comprises detecting (i) a single nucleotide polymorphism (SNP) in a Brd4 gene of the subject or (ii) an expression level of a Brd4 gene in the subject.

Furthermore, the invention provides methods of determining the metastatic capacity of a tumor. In one method, the method comprises detecting (i) a single nucleotide polymorphism (SNP) in an Anakin gene of the subject, (ii) an amino acid substitution in an Anakin protein in the subject, or (iii) an expression level of an Anakin gene in the subject, wherein detection of (i) or (ii) or an under-expression of the Anakin gene is indicative of a high metastatic capacity of the tumor in the subject. In another method, the method comprises detecting (i) a SNP in a Brd4 gene of the subject or (ii) an expression level of a Brd4 gene in the subject, wherein detection of (i) or an under-expression of the Brd4 gene is indicative of a high metastatic capacity of the tumor in the subject.

With respect to the inventive methods involving detecting an expression level of an Anakin gene or a Brd4 gene, a variety of techniques known in the art can be used to detect an expression level of the Anakin gene or Brd4 gene. For example, Western blotting can be used to compare the levels of Anakin protein or Brd4 protein expressed in two different cell populations. Alternatively, Northern blotting can be used to compare the levels of Anakin mRNA or Brd4 mRNA expressed in two different cell populations. Finally, Southern blotting can be used to compare the number of copies of the Anakin gene or Brd4 gene found in two different cell populations. These processes are described in Sambrook et al. (2001), supra. In a preferred embodiment of the inventive method of detecting cancer or a predisposition to cancer, detecting an expression level of an Anakin gene or Brd4 gene comprises detecting a level of Anakin mRNA or Anakin protein, or Brd4 mRNA or Brd4 protein.

With respect to the inventive methods involving detection of an amino acid substitution in an Anakin protein, any suitable method of detecting an amino acid substitution in a protein known in the art can be used. For example, a method comprising comparing by way of using the BLAST2sequences software program available at the NCBI website a given sequence suspected to have an amino acid substitution to an Anakin amino acid sequence, e.g., a human Anakin amino acid sequence, can be used. Alternatively, immunoassays using an antibody specific for a particular amino acid substitution in an Anakin protein can be used.

In this regard, the invention further provides an antibody, or antigen binding portion thereof, which specifically binds to a murine Anakin protein or an Anakin allelic variant. The murine Anakin protein to which the antibody or antigen binding portion thereof binds can be any murine Anakin protein as described herein. Preferably, the murine Anakin protein comprises the amino acid sequence of SEQ ID NO: 3. More preferably, the antibody or antigen binding portion thereof does not cross-react with a human Anakin protein, (e.g., SEQ ID NO: 1). For example, the antibody or antigen binding portion thereof can bind to an epitope of the murine Anakin protein which is unique to the murine Anakin. The Anakin allelic variant can be any allelic variant encoded by any allele containing an Anakin gene. Preferably, the Anakin allelic variant comprises the amino acid sequence of SEQ ID NO: 1 with an amino acid substitution of Leu to Pro at position 436 of SEQ ID NO: 1. In a more preferred embodiment, the antibody or antigen binding portion thereof binds to an epitope comprising the amino acid at position 436 of the wildtype Anakin amino acid sequence (SEQ ID NO: 1) or of the Anakin allelic variant.

The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the murine Anakin protein or Anakin allelic variant. Desirably, the antibody is specific for the murine Anakin protein or Anakin allelic variant, such that there is minimal cross-reaction with other peptides or proteins.

Methods of testing antibodies for the ability to bind to a murine Anakin protein or Anakin allelic variant are known in the art and include any antibody-antigen binding assay, such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display furthermore can be used to generate the antibody of the invention. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

The invention also provides antigen binding portions of any of the antibodies described herein. The antigen binding portion can be any portion that has at least one antigen binding site, such as Fab, F(ab′)2, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)). Antibody fragments of the invention, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The inventive antibodies and antigen binding portions can be packaged as a component of a kit. In this regard, the invention further provides a kit comprising any of the antibodies or antigen binding portions described herein and a set of user instructions. The kit can further comprise additional agents or materials, such as a vial of antibodies specific for a wildtype Anakin protein and a vial of antibodies specific for an Anakin allelic variant.

With respect to the inventive methods involving detection of a SNP in an Anakin gene or a Brd4 gene, the SNP can be a base transition or a base transversion. For purposes herein, the term “single nucleotide polymorphism” or “SNP” is defined as an inter-individual, single nucleotide variation in a genetic sequence that occurs at appreciable frequency in a population. More specifically, a SNP is a single-base nucleotide substitution that can result from a base transition (A for G, T for C) or base transversion (G or A for T or C). Also, the SNP can be one that results in an amino acid substitution, for example, a leucine to proline substitution. The amino acid substitution can be a conservative or non-conservative amino acid substitution. The amino acid substitution can be one that leads to a mutant protein having a different biological function (catalytic activity, binding activity, subcellular localization, etc.) and/or a different activity level when compared to the wildtype protein. Alternatively, the single nucleotide polymorphism can be a silent polymorphism, e.g., one that does not result in an amino acid substitution. In a preferred embodiment of the invention, the SNP results in an amino acid substitution. In a more preferred embodiment, the amino acid substitution is a Leu substituted for a Pro at position 436 of the human Anakin gene (SEQ ID NO: 1).

The SNP can be located in any region of the Anakin gene or Brd4 gene, e.g., an exon, an intron, the 5′ untranslated region (UTR), the 3′ UTR, the promoter, the polyA tail, etc. The Anakin and Brd4 genes are known in the art; the sequences of which are available as described herein.

Preferably, the SNP is located within the promoter of the Anakin gene, within the exon of the Anakin gene, or within both, e.g., a first SNP is located within the promoter and a second SNP is located within an exon of the Anakin gene. The exon can be any exon of the Anakin gene. For instance, the exon can be one of Exons 1-16. Preferably, the exon can be Exon 13 of the Anakin gene. For example, the SNP can be a T→C at position 1421 of the human Anakin gene (SEQ ID NO: 2). Also, the SNP can be an insertion of A after nucleotide position 1540 or an insertion of A after nucleotide position −1132, wherein the nucleotide A of the ATG translation initiation site is +1. Detection of such SNPs can also be achieved through detection of the complementary SNP on the noncoding strand of the human Anakin gene. For instance, if the SNP is a T→C polymorphism on the coding strand, then the complementary SNP would be A→G on the noncoding strand. In this regard, the SNP also can be a SNP that is complementary to the T→C SNP at position 1421 of the human Anakin gene.

With respect to Brd4, the SNP preferably is located within the human Brd4 gene, which gene is located within human chromosome 19. Preferably, the SNP is located within an intron of the human Brd4 gene. As such, the SNP in the Brd4 gene does not result in an amino acid substitution. The intron of the Brd4 gene can be any intron of the Brd4 gene. For instance, the intron can be one of Introns 1 to 18, e.g., Intron 6, Intron 9, Intron 10, Intron 11, Intron 13, and Intron 15. Preferably, the SNP is a SNP at position 15224477 of human chromosome 19 (position 14290 of SEQ ID NO: 112), a SNP at position 15213372 of human chromosome 19 (position 3185 of SEQ ID NO: 112), or a SNP at position 15224052 of human chromosome 19 (position 13,865 of SEQ ID NO: 112). More preferably, the SNP is an A→G SNP at position 15224477 of human chromosome 19 (position 14290 of SEQ ID NO: 112), a G→A SNP at position 15213372 of human chromosome 19 (position 3185 of the SEQ ID NO: 112), or a G→T SNP at position 15224052 of human chromosome 19 (position 13865 of SEQ ID NO: 112). Such SNPs are published in the dbSNP database of the NCBI website as Accession Nos. rs8104223, rs4808272, and rs11880801, respectively. Most preferably, the SNP is a G→T SNP at position 15224052 of human chromosome 19 (position 13865 of SEQ ID NO: 112. Detection of such SNPs can also be achieved through detection of the complementary SNP on the opposite strand of the human Brd4 gene. For instance, the complementary SNP of the A→G SNP would be a T→C SNP on the complementary (opposite) strand.

The SNPs described herein can be detected on one or both copies of the Anakin gene of a subject or on one or both copies of the Brd4 gene of a subject. In this regard, the subject can be described as heterozygous or homozygous for the SNP. If a subject is said to be heterozygous for the T→C SNP at position 1421 of the human Anakin gene, for example, it is meant that the subject has only one copy of the Anakin gene with the T→C variation, while the other copy of the Anakin gene in the subject does not have the T→C variation. Rather, the other copy has a T at that nucleotide position. For a subject that is homozygous for a given SNP, it is meant that both copies of the Anakin gene in that subject have the SNP or variation at the specified nucleotide position.

Methods of detecting a SNP are known in the art (see, for instance, Li et al., Nucleic Acids Research, 28(2): e1 (i-v) (2000); Liu et al., Biochem Cell Bio 80: 17-22 (2000); and Burczak et al., Polymorphism Detection and Analysis, Eaton Publishing, 2000). Suitable methods include, for instance, cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP) restriction fragment length polymorphism (RFLP), and direct DNA sequencing. Preferably, a method of detecting a SNP comprises a PCR reaction using gene-specific primers and SNP-specific probes. One illustration of such a method is described herein as Example 7. The SNP-specific probe is preferably labeled for detection. Suitable labels for probes are known in the art and include, for example, radioactive labels and fluorochromes, e.g., VIC (Applied Biosystems®), carboxy fluorescein (FAM), and 6-carboxy-tetramethyl-rhodamine (TAMRA). Preferred primers and probes to be used in the inventive methods involving detection of an Anakin SNP are disclosed herein as SEQ ID NOs: 5 to 8.

In this respect, the invention also provides a nucleic acid comprising a nucleotide sequence selected from the group consisting of SEQ ID NOs: 5 to 8.

The nucleic acids of the invention or of the inventive pharmaceutical compositions can be single-stranded or double-stranded, synthesized or obtained from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. The term “oligonucleotide” or “nucleic acid” as used herein means a polymer of DNA or RNA, (i.e., a polynucleotide).

With respect to the nucleic acids of the invention or of the inventive pharmaceutical compositions, it is preferred that no insertions, deletions, inversions, and/or substitutions are present. However, it may be suitable in some instances for the nucleic acids of the invention or of the inventive pharmaceutical compositions to comprise one or more insertions, deletions, inversions, and/or substitutions.

The nucleic acids of the invention or of the inventive pharmaceutical compositions can be constructed based on chemical synthesis and/or enzymatic ligation reactions using procedures known in the art. See, for example, Sambrook et al., Molecular Cloning: A Laboratory Manual, 3^{rd} Ed., Cold Spring Harbor Press, Cold Spring Harbor, N.Y. (2001) and Ausubel et al., Current Protocols in Molecular Biology, Greene Publishing Associates and John Wiley & Sons, New York, N.Y. (1994). For example, an oligonucleotide can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acid molecules, siRNA molecules, and shRNA molecules include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3) w, and 2,6-diaminopurine. Alternatively, one or more of the oligonucleotides of the present invention can be purchased from companies, such as Macromolecular Resources (Fort Collins, Colo.) and Synthegen (Houston, Tex.).

The nucleic acids of the invention or of the inventive pharmaceutical compositions can be modified to comprise a detectable label. The detectable label can be, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The nucleic acids of the invention can be packaged as a component of a kit. In this regard, the invention further provides a kit comprising a nucleic acid which specifically hybridizes to a portion of a nucleic acid comprising a nucleotide sequence encoding an Anakin protein or Anakin allelic variant and a set of user instructions. With respect to the kit of the invention, the Anakin protein can comprise the amino acid sequence of SEQ ID NO: 1 or 3, while the nucleic acid comprising a nucleotide sequence encoding an Anakin protein can comprise the nucleotide sequence of SEQ ID NO: 2 or 4. Also, the Anakin allelic variant can comprise the amino acid sequence of SEQ ID NO: 1 with an amino add substitution of Leu to Pro at position 436 of SEQ ID NO: 1. Further, the nucleic acid comprising a nucleotide sequence encoding an Anakin allelic variant can comprise the nucleotide sequence of SEQ ID NO: 2 with a T→C SNP at position 1421 of SEQ ID NO: 2. Furthermore, the nucleic acid which specifically hybridize to the specified nucleic acid can be, for instance, the nucleic acids comprising the nucleotide sequence of SEQ ID NOs: 5 to 8. The kit can further comprise additional agents or materials, such as a reagents used in a PCR, a vial of antibodies specific for a wildtype Anakin protein, and a vial of antibodies specific for an Anakin allelic variant.

The inventive methods of detecting cancer or a predisposition to cancer, methods of determining the metastatic capacity of a tumor, characterizing a tumor or a cancer, and a method of determining a treatment for a subject afflicted with a tumor or cancer can be performed in vitro or in vivo. For example, the method can comprise detecting in an in vitro sample obtained from a subject (i) a SNP in an Anakin gene or a Brd4 gene of a subject, (ii) an amino acid substitution in an Anakin protein in a subject, or (iii) a level of expression of an Anakin gene or a Brd4 gene in a subject. Alternatively, the detecting can occur in vivo by for example, administering a labeled oligonucleotide primer, e.g., a radioactive oligo, that hybridizes to a SNP in an Anakin gene or a Brd4 gene, an Anakin nucleic acid molecule encoding an amino acid substitution in an Anakin protein, or a wild-type Anakin or Brd4 gene. Preferably, the method of detecting cancer or a predisposition to cancer is performed in vitro.

With respect to the methods involving detection of (i) an Anakin SNP or Brd4 SNP, (ii) an amino acid substitution in an Anakin protein, or (iii) an expression level of an Anakin gene or Brd4 gene, the method can further comprise comparing (i) the nucleotide sequence of the Anakin gene or Brd4 gene of the subject, (ii) the amino acid sequence of the Anakin protein of the subject, or (iii) the expression level of the Anakin gene or Brd4 gene in the subject to a control. The control can be, for example, (i) a nucleotide sequence of the Anakin gene or Brd4 gene, (ii) an amino acid sequence of the Anakin protein, or (iii) an expression level of the Anakin gene or a Brd4 gene of a subject that is known as “normal” or disease-free, e.g., known to not be afflicted with cancer. Alternatively, the control can be (i) a nucleotide sequence of the Anakin gene or Brd4 gene, (ii) an amino acid sequence of the Anakin protein, or (iii) an expression level of the Anakin gene or Brd4 gene of a subject that is known as “abnormal” or diseased, e.g., known to be afflicted with cancer. Additionally or alternatively, the control can be (i) a nucleotide sequence of the Anakin gene or Brd4 gene, (ii) an amino acid sequence of the Anakin protein, or (iii) a level of expression of the Anakin gene or Brd4 gene of a population of subjects that are known to be “normal” or “abnormal.” For instance, the control can be a database containing information on (i) the nucleotide sequences of the Anakin gene or Brd4 gene, (ii) the amino acid sequences of the Anakin protein, or (iii) the levels of expression of the Anakin gene or Brd4 gene of the subjects of the population.

Further, in such methods involving detection of (i) an Anakin SNP or Brd4 SNP, (ii) an amino acid substitution in an Anakin protein, or (iii) a level of expression, e.g., an under-expression, of an Anakin gene or Brd4 gene, the tumor can be a tumor of any cancer, such as any of the cancers described herein, while the cancer can be any cancer, such as any of the cancers described herein. The cancer can be an epithelial cancer, e.g., a breast cancer, a prostate cancer, or a renal cell carcinoma. Preferably, the epithelial cancer is breast cancer or renal cell carcinoma. The cancer alternatively can be a non-epithelial cancer. Preferably, the cancer or tumor is a metastatic tumor or a metastatic cancer. The metastatic cancer can be any type of cancer as discussed herein.

The invention further provides methods of screening a compound for anti-cancer activity. In one method, the method comprises (a) providing a cell that (i) under-expresses an Anakin gene or (ii) comprises an Anakin allelic variant, (b) contacting the cell with a compound of interest, and (c) assaying for anti-cancer activity. In another method, the method comprises (a) providing a cell that (i) under-expresses a Brd4 gene or (ii) comprises a Brd4 allelic variant, (b) contacting the cell with a compound of interest, and (c) assaying for anti-cancer activity.

Also, the invention provides use of a compound with anti-cancer activity for the preparation of a medicament to treat or prevent cancer in a subject who has been tested for (i) a single nucleotide polymorphism (SNP) in an Anakin gene of the subject, (ii) an amino acid substitution in an Anakin protein in the subject, or (iii) an expression level of an Anakin gene in the subject.

Further provided is the use of a compound with anti-cancer activity for the preparation of a medicament to treat or prevent cancer in a subject who has been tested for (i) a single nucleotide polymorphism (SNP) in a Brd4 gene of the subject or (ii) an expression level of a Brd4 gene in the subject.

The anti-cancer activity can be any anti-cancer activity, including, but not limited to the reduction or inhibition of any of uncontrolled cell growth, loss of cell adhesion, altered cell morphology, foci formation, colony formation, in vivo tumor growth, and metastasis. Suitable methods for assaying for anti-cancer activity are known in the art (see, for example, Gong et al., Proc Nad Acad Sci USA, 101(44):15724-15729 (2004)—Epub 2004 Oct. 21; and Examples 3 and 4 set forth below.)

The compound can be any compound, including, but not limited to a small molecular weight compound, peptide, peptidomimetic, macromolecule, natural product, synthetic compound, and semi-synthetic compound. With respect to the inventive method of screening, the method can comprise screening more than one compound of interest simultaneously or separately. For example, the method can comprise screening a library of compounds with cells under-expressing an Anakin gene. Such libraries, e.g., small molecular weight compound libraries, are known in the art and are available from organizations, including, but not limited to the National Cancer Institute. Preferably, the method comprises screening more than one compound at a time. With respect to the inventive use of the compound, the compound can be a compound known to have anti-cancer activity, such as, for instance, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. Alternatively, the compound can be a compound identified through the inventive method of screening.

For purposes herein, the cancer can be any cancer. As used herein, the term “cancer” is meant any malignant growth or tumor caused by abnormal and uncontrolled cell division that may spread to other parts of the body through the lymphatic system or the blood stream. The cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

The cancer can be an epithelial cancer. As used herein the term “epithelial cancer” refers to an invasive malignant tumor derived from epithelial tissue that can metastasize to other areas of the body, e.g., a carcinoma. Preferably, the epithelial cancer is breast cancer or renal cell carcinoma. Alternatively, the cancer can be a non-epithelial cancer, e.g., a sarcoma, leukemia, myeloma, lymphoma, neuroblastoma, glioma, or a cancer of muscle tissue or of the central nervous system (CNS).

The cancer can be a non-epithelial cancer. As used herein, the term “non-epithelial cancer” refers to an invasive malignant tumor derived from non-epithelial tissue that can metastasize to other areas of the body.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein. Preferably, the cancer is a metastatic cancer.

As used herein, the term “subject” is meant any living organism. Preferably, the subject is a mammal. The term “mammal” as used herein refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is further preferred that the mammals are from the order Artiodactyla, including Bovines (cows) and Swines (pigs) or of the order Perssodactyla, including Equines (horses). It is further preferred that the mammals are of the order Primates, Ceboids, or Simoids (monkeys) or of the order Anthropoids (humans and apes). An especially preferred mammal is the human.

The nucleic acids of the invention or of the inventive pharmaceutical compositions and inventive antibodies can be isolated, purified, and/or synthetic. The term “isolated” as used herein means having been removed from its natural environment. The term “purified” as used herein means having been increased in purity, wherein “purity” is a relative term, and not to be necessarily construed as absolute purity. The term “synthetic” refers to partially or wholly synthesized materials.

The data presented herein further supports that the Anakin protein can inhibit the Sipa-1 GTPase catalytic activity. Sipa-1 (also known in the art as Spa-1) was originally cloned as a mitogen-inducible protein (Hattori et al., Mol. Cell. Biol., 15(1): 552-560 (1995)) that was subsequently shown to be a negative regulator of Rap-1 (Kurachi et al., J. Biol. Chem., 272(44): 28081-28088 (1997)). Sipa-1 has been shown to have significant effects on cellular adhesion (Tsukamoto et al., J. Biol. Chem., 274(26): 18463-18469 (1999)) and has been demonstrated to have effects on cell cycle progression (Hattori et al., supra): Yajnik et al., Cell, 112(5): 673-684 (2003)). Sipa-1 has recently been shown to interact with a bromodomain protein, Brd4, and alterations in the relative ratio of these two proteins disrupted normal cell cycle proliferation (Yajnik et al., supra). The Sipa-1 homozygous knockout animals are viable but eventually develop a myeloproliferative stem cell disorder (Farina et al., Mol. Cell. Biol., 24(20): 9059-9069 (2004)). The amino acid sequence of the Sipa-1 protein is available from the GenBank database (Accession number NP_{—}694985 or NP_{—}006738 (human) and NP_{—}035509 (mouse)). Further, it has been shown that metastatic capacity correlates with cellular Sipa-1 levels (Park et al., Nature Genetics, epublication on Sep. 4, 2005) and that a polymorphism in the region of the Sipa-1 gene which encodes the PDZ domain correlates with high metastatic potential (Park et al., 2005, supra).

In this regard, the invention provides a method of inhibiting Sipa-1 in a subject in need thereof. The method comprises administering to the subject (i) a nucleic acid comprising a nucleotide sequence encoding an Anakin protein, (ii) a vector comprising the nucleic acid, (iii) a host cell comprising the vector, (iv) an Anakin gene product, or (v) a combination thereof. The nucleic acid can comprise the nucleotide sequence of SEQ ID NO: 2 or 4. The Anakin gene product can be an Anakin protein (e.g., a protein comprising the amino acid sequence of SEQ ID NO: 1 or 3) or an Anakin mRNA. Preferably, the method effectively inhibits Sipa-1 GTPase activity. Methods of measuring GTPase activity are known in the art and include the method described herein in Example 2.

The terms “inhibit,” “prevent,” “reduce,” and “treat,” as well as words stemming therefrom, as used herein, do not necessarily imply 100% or complete inhibition, prevention, reduction, or treatment. Rather, there are varying degrees of inhibition, prevention, reduction, or treatment of which one of ordinary skill in the art recognizes as having a potential benefit or therapeutic effect. For purposes herein, the term “prevent” also includes the delaying the onset of the disease being prevented. In this respect, the inventive methods can provide any amount of prevention or inhibition of metastasis of a cancer cell, any level of prevention or inhibition of tumor growth, or any degree of prevention or treatment of a cancer in a subject.

### EXAMPLES

The following examples further illustrate the invention but, of course, should not be construed as in any way limiting its scope.

The following cells and reagents are used in the examples described herein:

The Mvt1 cell line was obtained as a gift from Lalage Wakefield (NCI, Bethesda). These cells are cultured in Dulbecco's Modification of Eagle's Medium (Cellgro, Va.) containing 10% fetal bovine serum (Cellgro, Va.), with culture medium being replaced at three day intervals. When the cells achieved confluency, they are washed once with 5 ml phosphate-buffered saline (PBS), incubated with 2 ml of trypsin-EDTA for 5 minutes, and passaged at a 1:30 dilution into a fresh culture flask.

### Example 1

This example demonstrates a method for identifying Sipa-1 binding partners.

The identification of Sipa-1 binding partners, especially those which bound to the PDZ domain of Sipa-1, is sought by performing a yeast two hybrid screen.

Yeast two hybrid screens using different regions of the human Sipa-1 protein. (Entrez Gene ID No: 6494) as bait are performed by ProNet technology (Myriad Genetics, Salt Lake City, Utah). The baits, which are used in the yeast two hybrid system, as well as the number of molecules shown to interact with the bait, are shown in Table 1.

TABLE 1

| | Amino Acid | | |
|---|---|---|---|
| | Coordinates | | Interactors |
| Bait Name | of Sipa-1 | Library(ies) Searched | Released |
| 16739_1 | 550 to 903 | Breast_cancer/Prostate_cancer, | 2 |
| | | Mouse_embryo, Spleen | |
| 16739_2 | 660 to 799 | Breast_cancer/Prostate_cancer, | 0 |
| | | Mouse_embryo, Spleen | |
| 16739_3 | 600 to 851 | Breast_cancer/Prostate_cancer, | 12 |
| | | Mouse_embryo, Spleen | |
| 16739_4 | 680 to 1030 | Breast_cancer/Prostate_cancer, | 2 |
| | | Mouse_embryo, Spleen | |
| 6411_3 | 170 to 350 | Brain, Spleen, Macrophage, | 0 |
| | | Breast_cancer/Prostate_cancer, | |
| | | Mouse_embryo | |
| 6411_4 | 340 to 550 | Brain, Spleen, Macrophage, | 0 |
| | | Breast_cancer/Prostate_cancer, | |
| | | Mouse_embryo | |
| 6411_7 | 850 to 1042 | Brain, Spleen, Macrophage, | 5 |
| | | Breast_cancer/Prostate_cancer, | |
| | | Mouse_embryo | |
| 6411_15 | −4 to 300 | Breast_cancer/Prostate_cancer, | 4 |
| | | Mouse_embryo, Spleen | |
| 6411_17 | 780 to 1043 | Breast_cancer/Prostate_cancer, | 6 |
| | | Mouse_embryo, Spleen | |
| 6411_31 | 750 to 903 | Breast_cancer/Prostate_cancer, | 3 |
| | | Mouse_embryo, Spleen | |
| 6411_32 | 278 to 560 | Mouse_embryo, | 1 |
| | | Breast_cancer/Prostate_cancer, | |
| | | Spleen | |
| 6411_33 | 250 to 361 | Mouse_embryo, | 0 |
| | | Breast_cancer/Prostate_cancer, | |
| | | Spleen | |

Thirty clones are found to bind to at least one of the Sipa-1 baits. The sequences of the clones are searched by the BLAST engine of the National Center of Biotechnology Information (NCBI) website. Table 2 lists the clones that are found to bind to at least one of the Sipa-1 baits.

TABLE 2

| Gene Symbol | Human Gene ID* | Mouse Gene ID* |
|---|---|---|
| Acin1 | 22985 | 56215 |
| ARPC3 | 10094 | 56378 |
| Calm2 | 805 | 12314 |
| Cdc42(191) | 998 | 12540 |
| EXOSC5 | 56915 | 27998 |
| Fasn | 2194 | 14104 |
| FLJ10276 | 55108 | 100383 |
| Gart | 2618 | 14450 |
| GTF2H2 | 2966 | 23894 |
| Itgb4(1805) | 3691 | 192897 |
| Kiaa0179 | 23076 | 72462 |
| LOC237422 | 55188 | 237422 |
| mAK078290 | 50944 | 243961 |
| mARRB1 | 408 | 109689 |
| mATP9A | 10079 | 11981 |
| mELMO2 | 63916 | 140579 |
| mKrt1-10 | 3858 | 16661 |
| mPLCB3 | 5331 | 18797 |
| mPRDX2 | 7001 | 21672 |
| mPRKAR1A | 5573 | 19084 |
| mSHANK3 | 85385 | 58234 |
| mUSP48 | 84196 | 362636 |
| NPC1 | 4864 | 18145 |
| Ric8b | 55188 | 237422 |
| s100A9 | 6280 | 20202 |
| Sipa1 | 6494 | 20469 |
| Snx2 | 6643 | 67804 |
| TNIP1(636) | 10318 | 57783 |
| Unc84B(717) | 25777 | 223697 |
| USF2 | 7392 | 22282 |

*Gene ID Nos. of the EntrezGene database of the NCBI website

A clone is found to bind to only the Sipa-1 baits comprising the PDZ domain of Sipa-1 (amino acids 683-752 of Sipa-1). This clone is sequenced by direct sequencing and the sequence is used to mine the Entrez Gene database. The search identifies this clone as the Riken clone (Entrez Gene ID No. 72462). Herein, the Riken clone is synonymous with Anakin.

The binding of Anakin to Sipa-1 is further confirmed by Western blotting immunoprecipitates of transfected cells. Specifically, COST cells are transiently co-transfected with pcDNA3 vector or pSRα-Sipa-1 expressing human Sipa-1, and pcDNA3 vector, pcDNA3-Aqp2, or pcDNA3-Anakin. Each dish receives the same total amount of DNA. Cells are transfected using lipofectamine (Invitrogen, Carlsbad, Calif.) according to the manufacturer's instructions. Two days after transfection, cells are lysed with Golden Lysis Buffer (GLB) containing 20 mM Tris, [pH 7.9], 137 mM NaCl, 5 mM EDTA, 1 mM EGTA, 10 mM NaF, 10% Glycerol, 1 mM sodium pyrophosphate, 1 mM Leupeptin, 1 mM PMSF and, aprotinin (10 μg/ml). Cell extracts are immunoprecipitated with anti-Sipa-1 mAb, anti-V5 antibody, or anti-Aqp2 antibody, and protein A/G (PIERCE) is added with overnight rotation at 4° C. The immune complexes are washed once with GLB, once with high salt HNTG (20 mM Hepes, 500 mM NaCl, 0.1% of Triton-X 100, 10% of Glycerol), and twice with low salt of HNTG (20 mM Hepes, 150 mM NaCl, 0.1% of Triton-X 100, 10% of Glycerol). The immune complexes are then analyzed by immunoblotting with anti-V5 antibody or anti-Aqp-2 antibody (Santa Cruz Biotechnology, Inc., Santa Cruz, Calif.). Cell extracts from transfectants are also analyzed for protein expression by immunoblotting with anti-V5 antibody or anti-Aqp-2 antibody. For each blot, horseradish peroxidase-conjugated anti-rabbit, anti-mouse or anti-goat immunogobulin G is used for the second reaction at a 1:10,000 dilution. Immune complexes are visualized by enhanced chemiluminescences with an ECL Kit from Amersham Biosciences, Piscataway, N.J.

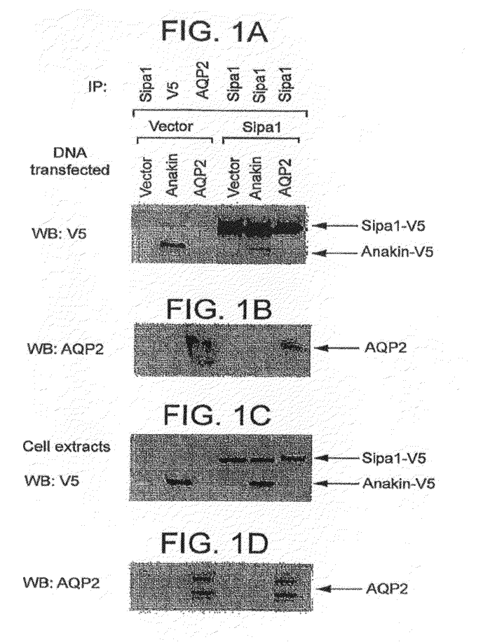

As shown in FIG. 1, Sipa-1 co-immunoprecipitates with Anakin only in cells expressing both Anakin and Sipa-1. Thus, the foregoing demonstrates that the Anakin protein binds to the PDZ domain of Sipa-1.

### Example 2

This example demonstrates that Anakin binding to Sipa-1 modulates the GTPase Activating Protein (GAP) activity of Sipa-1.

Because it is demonstrated that Anakin binds to the PDZ domain of Sipa-1 and since a Sipa-1 polymorphism in the region of the Sipa-1 gene which encodes the PDZ domain of Sipa-1 is shown to affect the GAP activity of Sipa-1, the effects of Anakin binding to Sipa-1 on the GAP activity of Sipa-1 is analyzed by a RalGDS pull-down assay as described in Park et al., 2005, supra. Briefly, COST cells are co-transfected as described in Example 1, except that a plasmid encoding Epac-HA (a guanine nucleotide exchange factor for Rap) is also added, to elevate the level of GTP•Rap-1. Two days after transfection, cells are processed using a Rap-1 activation kit (Upstate Biotech. Inc., Charlottesville, Va.), according to manufacturer's instructions. GTP•Rap-1 protein is pulled-down by RalGDS beads, washed three times, and subjected to gel analysis and immunoblotting with an anti-Rap-1 antibody (Santa Cruz). Cell extracts from transfectants are also analyzed as above for protein expression by immunoblotting with an anti-Rap1 antibody or anti-HA antibody (Convance, Inc., Princeton, N.J.).

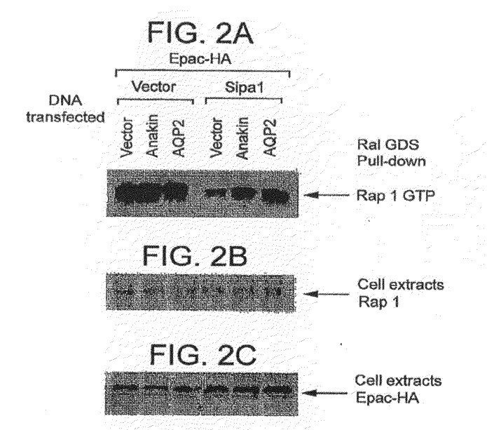

As shown in FIG. 2, Rap1GTP levels are dramatically increased in cells expressing both Anakin and Sipa-1 as compared to cells expressing Sipa-1 alone. Also, cells expressing both AQP2 and Sipa-1 exhibit a much higher level of Rap1GTP as compared to cells expressing Sipa-1 alone. Cells expressing Anakin or AQP2 but not expressing Sipa-1 are shown to have the same amounts of Rap1GTP as cells transfected with empty vectors.

The foregoing demonstrates that Anakin or AQP2 binding to Sipa-1 inhibits the GAP activity of Sipa-1.

### Example 3

This example demonstrates a method of identifying candidate ECM/metastasis modifier genes.

Microarray expression analysis is performed on mammary tumors derived from the F1 progeny of AKXD recombinant inbred mice crossed with the PyMT metastatic breast cancer model. Specifically, total RNA extractions from tissue samples are carried out using TRIzol® Reagent (Life Technologies, Inc., Gaithersburg, Md.) according to the standard protocol. Total RNA is prepared from whole blood using QIAamp RNA blood mini kit (Qiagen, Valencia, Calif.) per manufacture's instruction. RNA quantity and quality are determined by the Agilent Technologies 2100 Bioanalyzer (Bio Sizing Software version A.02.01., Agilent Technologies) and/or the GeneQuant Pro (Amersham Biosciences). Samples containing high-quality total RNA with A₂₆₀/A₂₈₀ ratios between 1.8 and 2.1 are purified with the RNeasy Mini Kit (Qiagen). An on-column genomic DNA digestion is performed as part of this purification step using the RNase-Free DNase Kit (Qiagen). Purified total RNA for each strain used in Affymetrix GeneChip assays is processed as previously described (Yang et al., Clinical and Experimental Metastasis 22: 593-603 (2005)). Hybridizations are performed on Affymetrix Murine Genome Moe430 A and B GeneChip® Arrays. Microarrays are processed using an Agilent GeneArray Scanner with Affymetrix Microarray Suite version 5.0.0.032 software. Three tumors from each of the 18 AKXD×PyMT outcross lines are assayed on the Affymetrix GeneChips. The data is uploaded to the web-based program WebQTL and normalized by either RMA or MAS5. The location of genomic regions associated with genetic modulation of ECM gene expression is determined by performing Interval Mapping analysis for each of the probe sets for the ECM genes. Identification of genes whose expression correlated with ECM gene expression is performed using the Trait Correlation function.

The microarray analysis identifies 7 genes: CentaurinD3 (CentD3); Csf1r, Brd4, Pi16, Luc7l, Necdin (Ndn), and 2600005C20Rik, herein referred to as Riken or Anakin.

Candidate genes for further evaluation as ECM/metastasis modifiers are chosen based on the following criteria: (1) the gene maps to an ECM eQTL interval; (2) the gene expression correlates with ECM gene expression; (3) the gene contains polymorphisms in the coding or promoter region of the gene; (4) in vitro ectopic expression alters endogenous ECM gene transcription; (5) in vitro ectopic gene expression alters metastasis in transplant assays; and (6) the gene is associated with metastatic breast cancer in human epidemiological studies.

The seven genes identified by the microarray analysis meet the second criteria, in that the gene expression of all seven genes correlate with the expression of four class predictive ECM genes, Fb1n2 (Entrez Gene ID No: 14115), Col1a1 (Entrez Gene ID No: 12842), Col5a3 (Entrez Gene ID No: 53867, and Serping1 (Entrez Gene ID No: 12258).

The seven genes identified by microarray analysis also meet the first criteria, as QTL mapping of the four microarray class prediction ECM genes are reproducibly observed on chromosomes 7, 17, and 18, which chromosomes are known to be important loci for metastasis genes. The eQTLs on chromosomes 17 and 18 co-localize with metastasis QTLs that are identified by performing composite interval mapping on the AKXD×PyMT experiment. In addition, chromosomal substitution strain analysis (replacement of the FVB chromosomes by NZB or ILn chromosomes by breeding) demonstrate the presence of metastasis modifiers on mouse chromosomes 7 and 17.

Because Ndn is shown in the literatures as a gene controlling collagen gene expression and since Anakin is shown to bind to Sipa-1, further studies focus on the Ndn and Anakin genes.

The foregoing demonstrates the identification of seven candidate ECM/metastasis modifier genes.

### Example 4

This example demonstrates the genes which are expressed in a correlative manner with the gene expression of the four class predictive ECM genes identified in Example 3.

Expression quantitative trait loci (eQTL) mapping of class-predictive ECM genes is performed to see if eQTLs co-segregate with metastasis QTLs. eQTL candidates which demonstrate reproducible associations with ECM gene expression across the AKXD panel are constructed into mammalian expression vectors. Expression vectors are obtained from the Mammalian Gene Collection, in pCMV-SPORT6, or by PCR. cloning into the vector pcDNA3.1-V5/His6. Those constructs that used the vector pcDNA3.1-V5/His6 are constructed using a pcDNA3.1/V5-His TOPO TA Expression Kit (Invitrogen, Carlsbad, Calif.). Briefly, PCR products are designed to amplify the gene of interest including the including the Kozak translation initiation codon, but excluding the native stop codon. PCR products are cloned into the vector DNA and transformed into competent E. Coli as per the manufacturer's instructions. Cells are grown overnight on a selective plate and individual transformant colonies are isolated and grown. Vector DNA is then extracted from each colony and insert ends are sequenced to identify those clones with correct insert orientation. Those clones with the insert correctly orientated are completely sequence verified before transfection.

The Mvt1 cell line (Pei et al., In Vitro Cell Dev Biol. Anim., 40 (1-2): 14-21 (2004)), derived from primary mammary tumor in an MMTV-VEGF/myc bi-trangenic mouse, is used to generate the stable cell lines expressing the different genes. Supercoiled plasmids are transfected into Mvt1 using Superfect Transfection Reagent (Qiagen, Valencia, Calif.). Those genes present in vectors obtained from the Mammalian Gene Collection (pCMV-Sport6) are co-transfected with the vector pSuper.Retro.Puro (Oligoengine) containing no insert as a selectable marker for transfectants. Twenty-four hours after transfection, the cells are selected in medium containing either 10 μg/ml puromycin (pCMV-Sport6/pSuper.Retro.Puro transfected cells) or 700 μg/ml neomycin (pcDNA3.1-V5/His6 transfected cells) and are transferred to 96 well plates and individual clones selected by limiting dilution. Colonies are screened either by quantitative PCR as described below or by Western blotting against V5 antibody as described above to identify clones expressing the gene of interest.

Quantitative PCR of the transfected cells is carried out. Specifically, mRNAs of the transfected cells are transcribed into cDNA using ThermoScript™ RT-PCR System (Invitrogen, Carlsbad, Calif.) by following its protocol. SYBR Green Quantitative PCR is performed to detect the mRNA levels of Brd4, Pi16, Luc7l, and Anakin genes using an ABI PRISM 7500 and/or 7900HT Sequence Detection Systems and custom designed primers (Table 2). Reactions are performed using QuantiTect SYBR Green Master Mix (Qiagen, Valencia, Calif.) as per the manufacturer's protocol. TaqMan Quantitative PCR is performed to detect the mRNA levels of CentD3 and Ndn genes using an ABI PRISM 7500 and/or 7900HT Sequence Detection Systems, with custom designed primers and probes labeled with the dye 5-(&6)-carboxyfluorescein (FAM) (Table 3). The gene Csf1r is detected using the Applied Biosystems Assay-On-Demand assay I.D. No. Mm00432689_m1. All TaqMan reactions are carried out using TaqMan Universal PCR Master Mix (Applied Biosystems, Foster City, Calif.). The mRNA level for each gene is normalized to peptidylprolyl isomerase B (Ppib) mRNA levels using either custom-designed primers for SYBR Green-amplified target genes (Table 3) or custom-designed primers and a FAM-labeled probe for TaqMan-amplified target genes (Table 4).

TABLE 3

| | | | SEQ |
|---|---|---|---|
| Gene | | | ID |
| Symbol | Primer Name | Sequence | NO |
| PpiB | Forward Primer | GGAGATGGCACAGGAGGAAAGAG | 27 |
| | Reverse Primer | TGTGAGCCATTGGTGTCTTTGC | 28 |
| Pi16 | Forward Primer | GGCCACTACACTCAGGTAGTGTGGA | 29 |
| | Reverse Primer | AGGCTCATAGTTGCACACCAGC | 30 |
| Anakin | Forward Primer | ACGCAGAGCGACACAGGAAG | 31 |
| | Reverse Primer | GCTCGTCCTGCACCCACA | 32 |
| Luc71 | Forward Primer | GAAGGAAATGTGGACGAATCCCAGA | 33 |
| | Reverse Primer | GCTGAACAAACCTCGCAAACACGTA | 34 |
| Brd4 | Forward Primer | GCTGAACCTCCCTGATTAC | 35 |
| | Reverse Primer | CATTCCTGAGCATTCCAGTA | 36 |

TABLE 4

| | | | SEQ |
|---|---|---|---|
| Gene | | | ID |
| Symbol | Oligo Name | Sequence | NO: |
| Necdin | Forward Primer | GTGGTACGTGTTGGTGAAGGA | 37 |
| | Reverse Primer | GTAGCTGCCCATGACCTCTT | 38 |
| | Probe | 6FAM-TCACCATGTCTGGAAACC | 39 |
| PpiB | Forward Primer | GGAGATGGCACAGGAGGAAAGAG | 40 |
| | Reverse Primer | TGTGAGCCATTGGTGTCTTTGC | 41 |
| | Probe | 6FAM-TCTATGGTGAGCGCTTC | 42 |
| CentD3 | Forward Primer | CCGGAGGACCTTATCCATGTT | 43 |
| | Reverse Primer | GCTCATCTTGCTCTTCCACAGA | 44 |
| | Probe | 6FAM-TTTCCAATGAAGTCACCC | 45 |

Ectopic expression of Necdin and Anakin cause significant expression changes in the 4 ECM genes identified in Example 3. Fibrillin and Col5a3. expression is down-regulated in cells ectopically expressing Anakin, whereas expression of Col1a1 is upregulated more than 5-fold the expression of a control cell line (Mvt-1 co-transfected with pCMV-Sport-β-Gal(Invitrogen, Carlsbad, Calif.) and pSuper.Retro.Puro). Also, Kai1/Cd82 gene expression is upregulated in cells expressing either Necdin or Anakin.

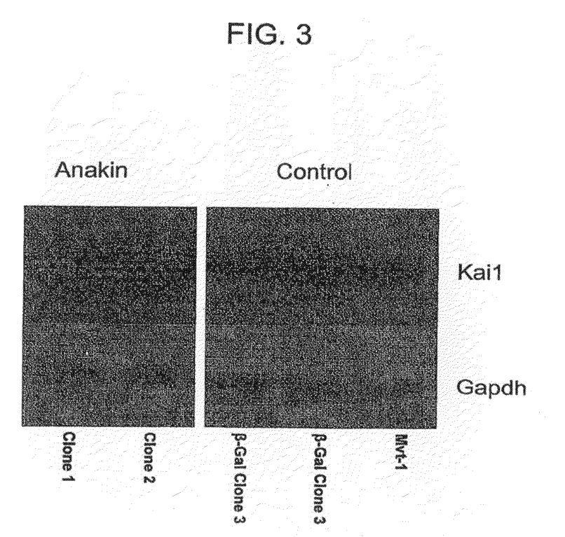

Whether or not the upregulation of Kai1/Cd82 expression in cells transfected with the Anakin gene leads to an increase in Kai1/Cd82 protein is next analyzed by Western blotting the Anakin-transfected cells using anti-Kai1 antibodies. As shown in FIG. 3, the protein levels of Kai1 are significantly increased in cells ectopically expressing Anakin, whereas the protein levels of GAPDH in the transfected cells are the same as that in untransfected cells.

The foregoing demonstrates that Anakin and Ndn are candidate ECM/metastasis modifiers.

### Example 5

This example demonstrates the reduction of tumor growth and metastasis in mice with implanted Mvt1 cells expressing Anakin or Ndn.

Stably transfected cells produced in Example 4 are subcutaneously implanted into virgin FVB/NJ mice. Two days before injection, cells are passaged and permitted to grow to 80-90% confluence. The cells are then washed with PBS and trypsinized, collected, washed twice with cold PBS, counted in hemocytometer and resuspended at a concentration of 106 cells/ml. One hundred thousand cells (100 μl) are injected subcutaneously in the vicinity of the fourth mammary gland of 6 week old virgin FVB/NJ female mice. The mice are then aged for 4 weeks before euthanization by anesthetic overdose. Tumors are dissected and weighted. Lungs are isolated and surface metastases are enumerated using a dissecting microscope. Tumor growth and metastasis are compared to mice injected with 10⁵ Mvt-1 cells stably co-transfected with pCMV-Sport-β-Gal and pSuper.Retro.Puro.

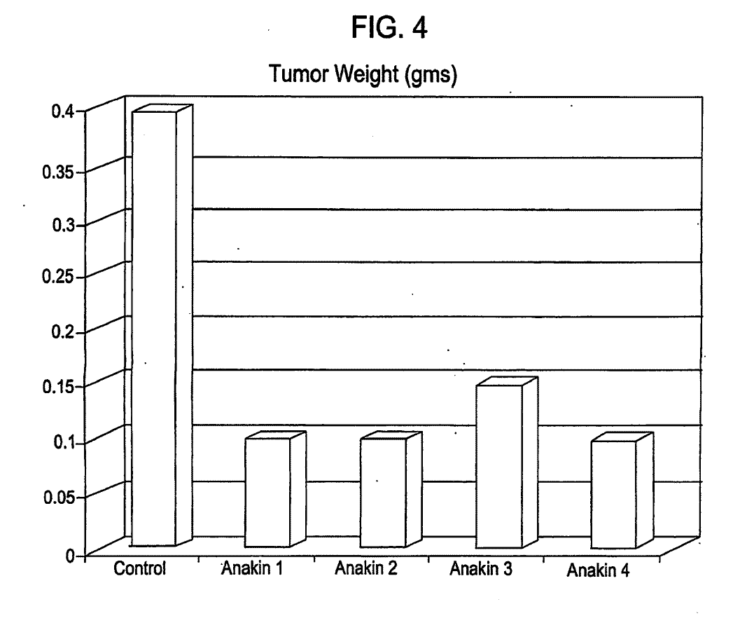

As shown in FIG. 4, the weight of tumors from mice with implanted Mvt1 cells stably expressing Anakin is significantly lower than the weight of tumors from control mice.

As shown in Table 5, the ectopic expression of Ndn suppresses tumor growth and metastasis.

TABLE 5

| | | Original Tumour | | Lung Surface | |
|---|---|---|---|---|---|
| Vector/Clone | Mouse ID | Weight (g) | | Metastasis Count | |
| pCMV Sport | 1 | | 0.1 | | 0 |
| Ndn/Clone 1 | 2 | | 0.2 | | 0 |
| | 3 | | 0.0 | | 0 |
| | 4 | | 0.0 | | 0 |
| | 5 | | 0.0 | | 0 |
| | 6 | | 0.1 | | 2 |
| | 7 | | 0.2 | | 0 |
| | 8 | | 0.1 | | 0 |
| | 9 | | 0.0 | | 0 |
| | | AVERAGE | 0.08 | AVERAGE | 0.22 |
| | | SD | 0.08 | SD | 0.67 |
| pCMV Sport | 1 | | 0.1 | | 0 |
| Ndn/Clone 4 | 2 | | 0.0 | | 0 |
| | 3 | | 0.0 | | 0 |
| | 4 | | 0.1 | | 0 |
| | 5 | | 0.1 | | 0 |
| | 6 | | 0.1 | | 0 |
| | 7 | | 0.0 | | 0 |
| | 8 | | 0.1 | | 2 |
| | 9 | | 0.0 | | 0 |
| | | AVERAGE | 0.06 | AVERAGE | 0.22 |
| | | SD | 0.05 | SD | 0.67 |
| pCMV Sport β- | 1 | | 0.7 | | 8 |
| Gal/Clone 4 | 2 | | 0.5 | | 5 |
| (Control cell line) | 3 | | 0.4 | | 10 |
| | 4 | | 0.6 | | 7 |
| | 5 | | 0.6 | | 17 |
| | 6 | | 0.7 | | 13 |
| | 7 | | 0.5 | | 8 |
| | 8 | | 0.6 | | 15 |
| | 9 | | 0.2 | | 5 |
| | | AVERAGE | 0.53 | AVERAGE | 9.78 |
| | | SD | 0.16 | SD | 4.32 |

The foregoing demonstrates that ectopic expression of Ndn leads to reduced metastasis and tumor growth, while Anakin leads to reduced tumor growth.

### Example 6

This example demonstrates that Anakin expression correlates with tumors with low metastatic capacity.

The expression of Ndn is analyzed in AKR and DBA tumors, which are tumors with high and low metastatic potential, respectively. Specifically, quantitative real time PCR is carried out as described in Example 4 in the cells of AKR and DBA tumors using the primers for Ndn as shown in Table 4. The copy number of Ndn in AKR tumor cells does not significantly differ from the copy number of Ndn in DBA tumor cells.

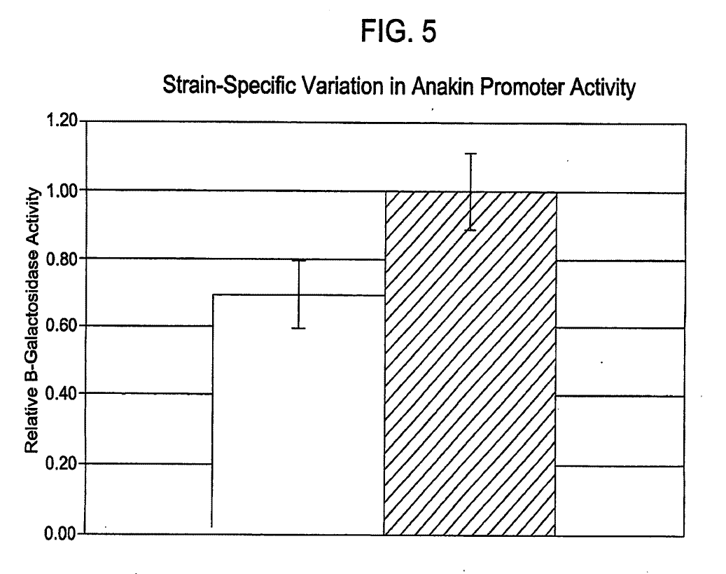

NIH-3T3 cells are transfected with a reporter plasmid comprising a nucleic acid encoding β-galactosidase (β-gal), with expression of β-gal being driven by either the AKR or DBA proximal Anakin promoter (pBlue-TOPO; Invitrogen). β-gal activity is assayed as described using a β-Galactosidase Assay Kit (Invitrogen). To normalize for transfection efficiency, cells are co-transfected with a luciferase reporter construct (pGL3-Control; Promega, Madison, Wis.) and luciferase activity assayed using a Dual Specificity Luciferase Assay Kit (Promega). As shown in FIG. 5, the cells transfected with the Anakin promoter from DBA tumors exhibited about 30% more β-gal activity than the cells transfected with the Anakin promoter from AKR tumors.

The foregoing demonstrates that low metastatic potential correlates with high or over-expression of Anakin.

### Example 7

This example demonstrates a method of detecting a SNP in Anakin and Ndn.

Complete sequencing of the exons, intron-exon boundaries and the promoters and regions immediately upstream of the promoters is performed in the two highly metastatic (AKR/J, FVB/NJ) and two low metastatic (DBA/2J, NZB/B1NJ) strains of mice (Park et al., Genome Res., 13(1): 118-121 (2003)). The sequences of the primers for Anakin are shown in Table 6 and for Ndn are shown in Table 7.

TABLE 6

| | | | | Product |
|---|---|---|---|---|
| Feature | | | SEQ | Length |
| Amplified | Primer | Sequence | ID NO: | (bp) |
| Promoter | Forward | AGTATGTTCCCGCTTGTG | 46 | 581 |
| | Reverse | ACTTGACTCTGTAAGTCCTGC | 47 | |
| Promoter | Forward | GGTCCTGGCTTCCTTCCAT | 48 | 606 |
| | Reverse | GGCTGACGACAGCACAGG | 49 | |
| Promoter, | Forward | AAAGAGCACGGCGGTAAG | 50 | 1600 |
| 5′-UTR, Exon 1 | Reverse | TTTCTTGCGTCTGCCTGG | 51 | |
| Exon 2 | Forward | GGAACATTAGCCATTAGCA | 52 | 440 |
| | Reverse | TGAAATGACGAGAGCAATAG | 53 | |
| Exon 3 | Forward | GCTTAGAGTTACACATTTGCTAA | 54 | 415 |
| | Reverse | AGAGTAACCTGAATGTGGAGA | 55 | |
| Exon 4 | Forward | GTAAGGACGCTCATCATC | 56 | 437 |
| | Reverse | AAAAGTGCCAGGTAAGTG | 57 | |
| Exon 5 | Forward | TTTGTTGGGCAGAGTCTATG | 58 | 426 |
| | Reverse | CAGGCGTAGGTCAGTCAAT | 59 | |
| Exon 6 | Forward | TCTTCTCTTGGGACCTCAC | 60 | 443 |
| | Reverse | GCAGTTCTGTCTACAAGTCCA | 61 | |
| Exon 7 | Forward | TCTGACCAGTTGGTGCTT | 62 | 386 |
| | Reverse | GAATGGGTGCTCCTTACAA | 63 | |
| Exon 8 | Forward | TGAATCTTGAGTGGACCTGC | 64 | 565 |
| | Reverse | TCTTCCAGGGCAATGAGG | 65 | |
| Exon 9 | Forward | GTGTTCTCCCTGGTAATGG | 66 | 370 |
| | Reverse | CCTTTCAACTGTGTCTCCAA | 67 | |
| Exon 10 | Forward | CTCCTCAGGCAGTTCTTCT | 68 | 349 |
| | Reverse | GCAAGAGCACACATACACAG | 69 | |
| Exon 11 | Forward | TGGAGGAGAGAGTGAGCA | 70 | 246 |
| | Reverse | CTTAGGTGAACGCAATGAG | 71 | |
| Exon 12 | Forward | GACAGTGGCAGGTAGTGC | 72 | 314 |
| | Reverse | AACCTGGGCTATGTGAGAC | 73 | |
| Exon 13 | Forward | CGGCAGACTTTAGACCAG | 74 | 414 |
| | Reverse | GCCCTCAGTTTCTTCTTTC | 75 | |
| Exon 13 | Forward | GCAAGCGTGTGTGACTGA | 76 | 403 |
| | Reverse | GGTGCTGGATGCTGTCTT | 77 | |
| Exon 13 | Forward | TGTCAGTGGGCATTCTCA | 78 | 501 |
| | Reverse | GAGATTGGAACCTGTCATTG | 79 | |
| Exon 14 | Forward | GCAGAGTTCCTGACAGAGC | 80 | 539 |
| | Reverse | TGATGTGGTGTTTGAGCC | 81 | |
| Exon 15 | Forward | ATTAGCCTTTGTGTGTGTGC | 82 | 322 |
| | Reverse | TGCCTAACTGACTAATCTGGA | 83 | |
| Exon 16 | Forward | TGTATCTTAGGTGTCTCCTGC | 84 | 527 |
| 3′-UTR | Reverse | ACCAACAGCACTCAGTCCT | 85 | |

TABLE 7

| Feature | | | SEQ | Product |
|---|---|---|---|---|
| Amplified | Primer | Sequence | ID NO: | Length (bp) |
| Promoter | Forward | ATTGGGAAAGATTTGGATGTGCTC | 86 | 626 |
| | Reverse | GTACCTTATGATGATGATGAGTTGTT | 87 | |
| Promoter, | Forward | CACTTTACATTCTTCCTTGTTTGA | 88 | 618 |
| Exon | Reverse | CAGGTCCTTACTTTGTTCCGA | 89 | |
| Promoter, | Forward | CTTCTGGCTTCCCAACACG | 90 | 741 |
| Exon | Reverse | GGGCATACGGTTGTTGAGC | 91 | |
| Exon | Forward | GTGAAGGACCAGAAGAGGATG | 92 | 598 |
| | Reverse | CAAGATTAGCCTCCCGCA | 93 | |
| Exon, | Forward | AGGAAGATAATCACCGAGGAGT | 94 | 585 |
| 3′-UTR | Reverse | CAGTCCCATACAAAGAACAAGATAC | 95 | |
| 3′-UTR | Forward | TGTGCTGTGCTAAACTTGTGAA | 96 | 614 |
| | Reverse | ATTCTGCTAAAGTGTCCATCAAA | 97 | |

PCR products are generated under standard amplification conditions (5 minutes at 94° C., 30 seconds at 57° C., 30 seconds at 72° C., and 5 minutes at 72° C.), purified with Qiagen PCR purification kits and double strand sequencing was performed with a Perkin Elmer BigDye Dye Terminator sequence kit. Analysis is performed on a Perkin Elmer 3100 Automated Fluorescent Sequencer. Sequences are compiled and analyzed with the computer software packages PHRED and PHRAP (Gordon et al., Genome Res., 8(3): 195-202 (1998)) to identify polymorphisms.

Haplotype variation of murine Anakin and Ndn (SEQ ID NOs: 3 and 11, respectively) is, in fact, observed between AKR and DBA tumor cells with SNPs in the promoter regions and coding regions of these two genes. The following polymorphisms are evident in the putative promoter of Anakin in the AKR strain when compared to DBA (polymorphisms are numbered where +1 is the “A” in the ATG translation initiation site): −1540ins(A); −1132ins(A).

The following polymorphisms are evident in the putative promoter of Ndn in the DBA strain when compared to AKR (polymorphisms are numbered where +1 is the “A” in the ATO translation initiation site): −997A→G; −804ins(AT); −503ins(CAT)₃; −336A→C; −137A→G. Additionally, the DBA strain displays a polymorphism in the coding region of Ndn (+50T→C) that results in a valine to alanine amino acid substitution in the translated Ndn protein (V18A).

Also, search of the Entrez Gene database identifies genes orthologous to Anakin. One ortholog is reported to have alternative splice variants, such that it is likely that the human Anakin gene is alternatively spliced.

The identification of human SNPs in these genes is next explored. Specifically, published SNPs within human Anakin and Ndn are searched for using the dbSNP database of the National Center for Biotechnology Information (NCBI) website. Four SNP entries are found for Anakin (Accession Nos. rs9306160, rs17292685, rs17845854, and rs17858827), while only one SNP entry is found for Ndn (Accession No. rs192206).

All SNP entries for Anakin report a T→C substitution at nucleotide position 1421 of the human Anakin gene (SEQ ID NO: 2). This SNP is found in the coding region of the gene and encodes a Leu to Pro amino acid substitution at amino acid position 436 of the human Anakin protein (SEQ ID NO: 1).

Anakin polymorphisms are characterized in the constitutional DNA derived from lymphocytes from breast cancer patients using SNP-specific polymerase chain reaction (PCR). PCR primers are designed using Vector NTI 9.0 software (Invitrogen, Carlsbad, Calif.) according to parameters described elsewhere (Crawford et al., Hum. Mutat. 25(2): 156-166 (2005)). Each probe is labeled with a reporter dye (either FAM [5-(&6)-carboxyfluorescein] or VIC® [a proprietary fluorescent dye produced by Applied Biosystems]) specific for wildtype and variant allele of Anakin, respectively. Sequences of PCR primers and fluorogenic probes are shown in Table 8.

TABLE 8

| | Sequence | SEQ ID NO: |
|---|---|---|
| Primer 1 | TGGACGTGGCCTCTGCAC | 98 |
| Primer 2 | CACCACCTGCAGCCTGAAA | 99 |
| Wildtype Probe | 6FAM-AGGGCTTTCAGCCCAGAG | 100 |
| Mutant Probe | VIC-AGGGCTTTCGGCCCAG | 101 |

Reaction mixtures consists of 300 nM of each oligonucleotide primer, 100 nM fluorogenic probes 8 ng template DNA, and 2× TaqMan Universal PCR Master Mix (Applied Biosystems, Foster City, Calif.) in a total volume of 10 μl. The amplification reactions are performed in a MJ Research DNA Engine thermocycler (Bio-Rad, Hercules, Calif.) with two initial hold steps (50° C. for 2 Min, followed by 95° C. for 10 min) and 40 cycles of a two-step PCR (92° C. for 15 sec, 60° C. for 1 min). The fluorescence intensity of each sample is measured post-PCR in an ABI Prism 7700 sequence detection system (Applied Biosystems, Foster City, Calif.), and Anakin SNP genotypes are determined by the fluorescence ratio of the nucleotide-specific fluorogenic probes.

Chi-square test of association is used to test for Hardy-Weinberg equilibrium. Chi-square and Fisher's exact test is used to test for differences between groups. Analysis of variance is performed in order to examine associations between the SNPs and continuous variables such as tumor size involvement of positive lymph nodes.

The breast cancer cases under study include 2 case groups (cases with localized disease [N=146] and cases with regional/metastatic disease [N=154]). Data in Table 9 show that the variant G allele in human Anakin appears to be protective, and its presence appears to correlate with indicators of improved outcome. Specifically, the presence of the G allele is associated with a lower frequency breast cancer with the following characteristics: distant metastatic disease (P=0.0057), tumors with a poor histological grade (P=0.0018), regional lymphatic metastasis, and primary tumors that do not express progesterone and/or estrogen receptors.

TABLE 9
Analysis of the rs9306160

| Genotype on | | | | | | | | |
|---|---|---|---|---|---|---|---|---|
| noncoding strand | Homozygous | | Heterozygous | | Homozygous | | | |
| of both alleles | GG | | AG | | AA | | Total | P_value |
| Stage | | | | | | | | |
| Metastatic | 17 | 43.6% | 22 | 56.4% | 0 | 0.0% | 39 | 0.0057 |
| Regional | 52 | 48.6% | 44 | 41.1% | 11 | 10.3% | 107 | |
| Local | 47 | 34.6% | 62 | 45.6% | 27 | 19.9% | 136 | |
| Grade | | | | | | | | |
| Poor | 50 | 50.0% | 45 | 45.0% | 5 | 5.0% | 100 | 0.0018 |
| Well to Moderate | 41 | 34.2% | 55 | 45.8% | 24 | 20.0% | 120 | |
| Presence + Nodes | | | | | | | | |
| Yes | 63 | 48.1% | 57 | 43.5% | 11 | 8.4% | 131 | 0.0072 |
| No | 43 | 33.6% | 59 | 46.1% | 26 | 20.3% | 128 | |
| Age at Diagnosis | | | | | | | | |
| <50 | 43 | 44.8% | 40 | 41.7% | 13 | 13.5% | 96 | 0.6318 |
| >=50 | 73 | 39.3% | 88 | 47.3% | 25 | 13.5% | 186 | |
| Progesterone | | | | | | | | |
| Receptor Status | | | | | | | | |
| − | 41 | 50.0% | 38 | 46.3% | 3 | 3.7% | 82 | 0.0026 |
| + | 61 | 35.9% | 77 | 45.3% | 32 | 18.8% | 170 | |
| Estrogen | | | | | | | | |
| Receptor Status | | | | | | | | |
| − | 28 | 52.8% | 25 | 47.2% | 0 | 0.0% | 53 | 0.0026 |
| + | 74 | 36.8% | 92 | 45.8% | 35 | 17.4% | 201 | |
| Tumor size | | | | | | | | |
| >2 cm | 45 | 39.1% | 57 | 49.6% | 13 | 11.3% | 115 | 0.3638 |
| <2 cm | 61 | 40.7% | 64 | 42.7% | 25 | 16.7% | 150 | |

The SNP entry for human Ndn reports a C→T substitution at nucleotide position 944 of the human Ndn gene (SEQ ID NO: 10). This SNP is found in the coding region of the gene and but does not encode an amino acid substitution in human Ndn protein (SEQ ID NO: 9). Ndn polymorphisms are characterized using SNP-specific polymerase chain reaction (PCR) as was performed for Anakin SNPs. Sequences of PCR primers and fluorogenic probes are shown in Table 10.

TABLE 10

| | Sequence | SEQ ID NO: |
|---|---|---|
| Primer 1 | GAAATCACCAAGATGCAAATCATG | 102 |
| Primer 2 | GGCCTCCTCCAGAGCTTCTC | 103 |
| Wildtype Probe | 6-FAM-AGAAAGACCCCCAGGCC | 104 |
| Mutant Probe | VIC-TTAAGAAAGATCCCCAGGCC | 105 |

As shown in Table 11, the Ndn SNP does not correlate with metastasis.

TABLE 11
Analysis of the rs2192206

| Genotype on | | | | | | | | |
|---|---|---|---|---|---|---|---|---|
| noncoding strand | Homozygous | | Heterozygous | | Homozygous | | | |
| of both alleles | GG | | AG | | AA | | Total | P_value |
| Stage | | | | | | | | |
| Metastatic | 26 | 65.0% | 7 | 17.5% | 7 | 17.5% | 40 | 0.9157 |
| Regional | 68 | 65.4% | 23 | 22.1% | 13 | 12.5% | 104 | |
| Local | 94 | 67.1% | 27 | 19.3% | 19 | 13.6% | 149 | |
| Grade | | | | | | | | |
| Poor | 63 | 64.3% | 20 | 20.4% | 15 | 15.3% | 98 | 0.7591 |
| Well to Moderate | 84 | 66.7% | 27 | 21.4% | 15 | 11.9% | 126 | |
| Presence + Nodes | | | | | | | | |
| Yes | 85 | 66.9% | 28 | 22.1% | 14 | 11.0% | 127 | 0.7680 |
| No | 89 | 66.9% | 26 | 19.6% | 18 | 13.5% | 133 | |
| Age at Diagnosis | | | | | | | | |
| <50 | 63 | 64.3% | 24 | 24.5% | 11 | 11.2% | 98 | 0.3289 |
| >=50 | 125 | 67.2% | 33 | 17.7% | 28 | 15.0% | 186 | |
| Estrogen | | | | | | | | |
| Receptor Status | | | | | | | | |
| − | 34 | 64.1% | 9 | 17.0% | 10 | 18.9% | 53 | 0.5218 |
| + | 138 | 68.0% | 39 | 19.2% | 26 | 12.8% | 203 | |
| Progesterone | | | | | | | | |
| Receptor Status | | | | | | | | |
| − | 54 | 65.9% | 13 | 15.9% | 15 | 18.3% | 82 | 0.3562 |
| + | 116 | 67.4% | 35 | 20.4% | 21 | 12.2% | 172 | |
| Tumor size | | | | | | | | |
| >2 cm | 76 | 64.4% | 25 | 21.2% | 17 | 14.4% | 118 | 0.6254 |
| <2 cm | 104 | 69.8% | 28 | 18.8% | 17 | 11.4% | 149 | |

The foregoing demonstrates that a SNP in the Anakin gene correlates with a protective characteristic of breast cancer. Specifically, a SNP in the Anakin gene is correlative with distant metastatic disease, tumors with a poor histological grade, regional lymphatic metastasis, and primary tumors that do not express progesterone and/or estrogen receptors breast cancer.

### Example 8

This example demonstrates a method of preventing or inhibiting tumor growth and metastasis by ectopic expression of Brd4.

Spontaneous metastasis assays are performed to assess the effect of ectopic expression of Brd4 on tumor growth and metastasis in the highly metastatic Mvt-1 cell line. The Mvt-1 cell line is obtained as a gift from Lalage Wakefield (NCI, Bethesda). Cells are cultured in Dulbecco's Modification of Eagle's Medium (DMEM; Cellgro, Va.) containing 10% fetal bovine serum (FBS; Cellgro, Va.), with culture medium being replaced at three day intervals. When the cells achieved confluency, the cells are washed once with 5 ml phosphate-buffered saline (PBS), incubated with 2 ml of trypsin-EDTA for 5 minutes, and passaged at a 1:30 dilution into a fresh culture flask.

Mvt-1 clonal isolates ectopically expressing Brd4 are developed. Specifically, supercoiled plasmids, either a previously described construct encoding full-length Brd4 (Crawford et al., Breast Cancer Res. 8: R16 (2006)) or a control plasmid (pCMV-SPORT-β-Galactosidase (Invitrogen)) are transfected into Mvt-1 using Superfect Transfection Reagent (Qiagen, Valencia, Calif.) as per the manufacturer's instructions. Briefly, transfections are performed in 100 mm diameter culture dishes, with 2×10⁶ Mvt-1 cells seeded 24 hr prior to transfection. The Brd4-pFLAG-CMV2 and pCMV-SPORT-β-Galactosidase vectors are co-transfected with the vector pSuper.Retro.Puro (Oligoengine) containing no insert as a selectable marker for transfectants. Cells in each culture vessel are transfected with a total of 20 μg vector DNA using Superfect at a 6:1 lipid to DNA ratio. Twenty-four hours after transfection, the cells are selected in normal growth medium containing 10 μg/ml puromycin (Sigma Aldrich), transferred to 96 well plates and individual clones selected by limiting dilution. Colonies are screened by quantitative PCR as described below to identify clones ectopically expressing Brd4.

Total RNA samples are isolated from cell culture samples using an RNeasy Mini Kit (Qiagen) with sample homogenization being performed using a 21G needle and syringe as per the manufacturer's protocol. All samples are subjected to on-column DNase digestion, and RNA quality and quantity determined by an Agilent Technologies 2100 Bioanalyzer (Bio Sizing Software version A.02.01., Agilent Technologies). Only those samples containing high-quality total RNA with A260/A280 ratios between 1.8 and 2.1 are used for further analysis.

cDNA is synthesized from RNA isolated from either primary tumor tissues or transfected cell lines using the ThermoScript RT-PCR System (Invitrogen, Carlsbad, Calif.) by following the manufacturer's protocol. Single RT-PCRs are performed for each Mvt-1 clonal isolate. SYBR Green Quantitative PCR is performed to detect the cDNA levels of Brd4 using an ABI PRISM 7500 and/or 7900HT Sequence Detection Systems. Primer sequences for Brd4 quantification are as follows: 5′-GCTGAACCTCCCTGATTAC-3′ (SEQ ID NO: 106) and 5′-CATTCCTGAGCATTCCAGTA-3′ (SEQ ID NO: 107). Reactions are performed using QuantiTect SYBR Green Master Mix (Qiagen, Valencia, Calif.) as per the manufacturer's protocol. The cDNA level of each gene is normalized to Peptidylprolyl Isomerase B (Ppib) cDNA levels using custom-designed primers for SYBR green-amplified target genes.

Transfected cells proven to be stably expressing Brd4 are subcutaneously implanted into virgin FVB/NJ mice. Two days before injection, cells are passaged and permitted to grow to 80-90% confluence. The cells are then washed with PBS and trypsinized, collected, washed twice with cold PBS, counted with a hemocytometer and resuspended at a concentration of 10⁶ cells/ml. One hundred thousand cells (100 μl) are injected subcutaneously near the fourth mammary gland of 6-week-old virgin FVB/NJ female mice. The mice are then aged for 4 weeks before euthanized by anesthetic overdose. Tumors are dissected and weighed. Lungs are isolated and surface metastases enumerated using a dissecting microscope. Tumor growth and metastasis are compared to mice injected with 10⁵ Mvt-1 cells stably co-transfected with pCMV-Sport-β-Gal and pSuper.Retro.Puro. These experiments are performed in compliance with the National Cancer Institute's Animal Care and Use Committee guidelines.

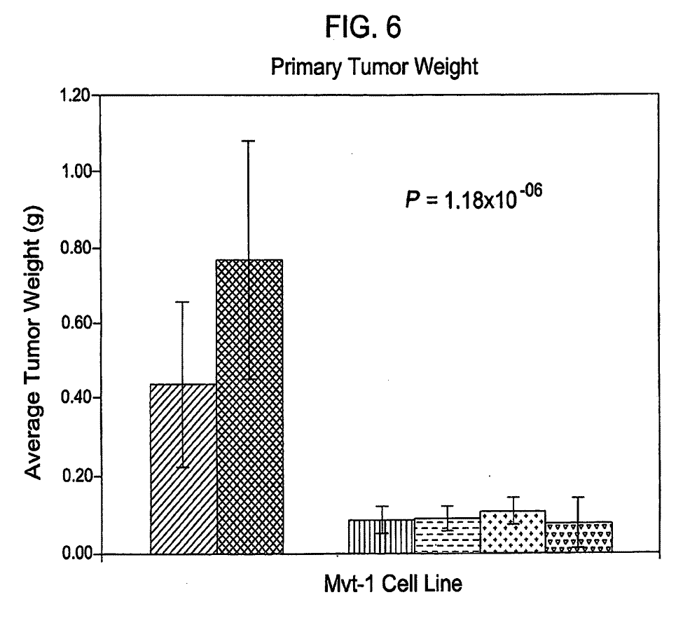

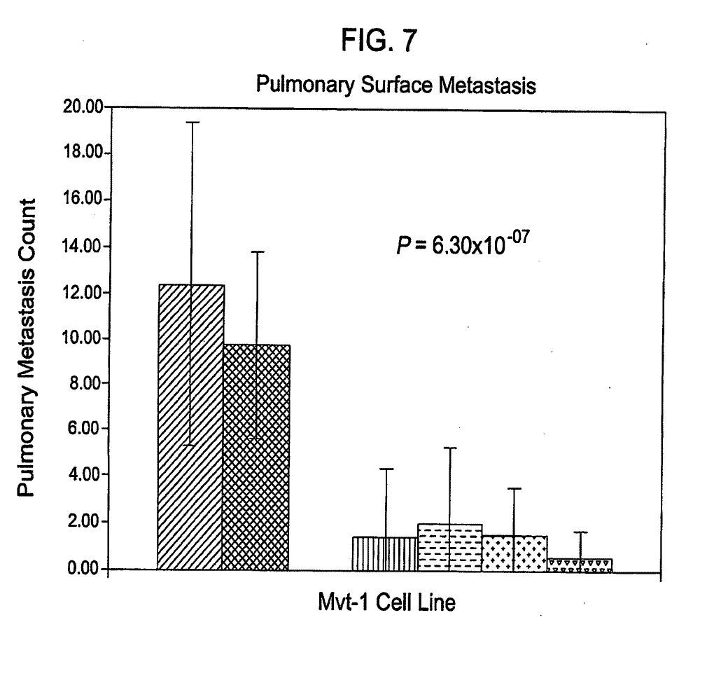

As shown in FIG. 6, tumor growth is significantly reduced in the four Mvt-1 clonal isolates ectopically expressing Brd4. The average tumor weight for the Mvt-1/Brd4 clones is 91 mg±42 mg compared to 595 mg±308 mg for the two Mvt-1/β-gal clones (P<0.001). As shown in FIG. 7, lung surface metastasis counts are significantly reduced in the four Mvt-1 clonal isolates ectopically expressing Brd4. The average lung surface metastasis count is 1.4±2.5 for the Mvt-1/Brd4 clones compared to 11.1±5.8 for the Mvt-1/β-gal clones (P<0.001). It is uncertain at present whether this reduction in metastatic capacity is dependent or independent of the reduced cellular growth kinetics observed in the Mvt-1/Brd4 clones. These data imply that activation of Brd4 is associated with a less malignant phenotype in the mouse.

This example demonstrated that tumor growth and metastatic potential are reduced by ectopic expression of Brd4.

### Example 9

This example demonstrates a method of detecting a SNP in Brd4.

Complete sequencing of the exons, intron-exon boundaries, the promoters, and regions immediately upstream of the promoters of the Brd4 gene is performed in two highly metastatic (AKR/J and FVB/NJ) and two low metastatic (DBA/2J, NZB/B1NJ) strains of mice as described in Example 7. The sequences of the primers for Brd4 are shown in Table 12.

TABLE 12

| Feature | | | SEQ | AKR vs. DBA |
|---|---|---|---|---|
| Amplified | Primer | Sequence | ID NO: | Polymorphism |
| Promoter | Forward | AGCCCAAAGTTAGACGCTTT | 113 | AKR: 631T > G |
| | Reverse | AGGTAGGCTGAGGCAGAAGG | 114 | Both: 641-642 Del |
| | | | | TT |
| | | | | AKR: 642insAAA |
| | | | | AKR: 695A > G |
| Promoter | Forward | TGCCTCAGCCTACCTTTTTC | 115 | |
| | Reverse | CCTTCTTGTCTCAGCCTTCC | 116 | |
| Promoter | Forward | ATGCTGGGAGCTGACTTACG | 117 | |
| | Reverse | AGGGAAGGAACCTTGCAGAT | 118 | |
| Promoter | Forward | GCTCAGTGGTAGAGCGCTTG | 119 | |
| | Reverse | CTCACCTGAGACGCTAGGC | 120 | |
| Promoter | Forward | GGCTGTTTGTTCTGCTCTCC | 121 | |
| | Reverse | CCTCCTCCTCCTCCTCACTT | 122 | |
| 5′-UTR | Forward | CGGAGCCTGGTGCTTCTC | 123 | |
| | Reverse | GAGTACCCAGCTGACGGAAG | 124 | |
| intron 1 | Forward | GCAGTTGGGAGCTGAGGTAG | 125 | |
| | Reverse | CTCTGGCCACACTGAAACAA | 126 | |
| intron 1 | Forward | TCTTGGTTCAGCAGGTCTCA | 127 | 2 bp intronic |
| | Reverse | GGTGTGATGACACAAACCAC | 128 | insertion-deletion |
| | | | | 1 bp intronic |
| | | | | insertion-deletion |
| intron 1 | Forward | GCCAAGACTGGCTTTGATCT | 129 | 1 bp insertion- |
| | Reverse | TGCCTGTTCTGTACCCTCAA | 130 | deletion |
| 5′-UTR | Forward | GAGAGGGTGGGGGTGATTAT | 131 | 1 bp insertion- |
| | Reverse | GCTGTGGACAATCTGAAGCA | 132 | deletion |
| | | | | SNP in 5′ UTR |
| 5′-UTR, | Forward | TACCAGTGGAGCCCAATCTT | 133 | |
| exon 1 | Reverse | CCCTGTCCAGATGGCTACTC | 134 | |
| Exon 2 | Forward | ACGTCTTTGGCTGTGGAGTT | 135 | |
| | Reverse | ACACCCAATCCTATGCACAA | 136 | |
| Intron 2 | Forward | GGCCATAAAATCCAGTGTCC | 137 | |
| | Reverse | CTGTCCCCGTTCAGCTCTAA | 138 | |
| Exon 3 | Forward | CTCCATGTATTGGAGCATGG | 139 | Intronic SNP |
| | Reverse | CATGGGACTTCCTAGGAGCA | 140 | |
| Exon 4 | Forward | CCTGAAGTGTTCCAGATGGTC | 141 | |
| | Reverse | GTCTCTGGTGGCAGCAATC | 142 | |
| Exon 5 | Forward | GGGCTTGTCCTGAGTATTGG | 143 | |
| | Reverse | CCCAGAACGTTGTTGGATTAG | 144 | |
| Exon 6 | Forward | GGAGTGATGGCCTGTTGTTC | 145 | |
| | Reverse | AGAACCAGCCACTCACATTTA | 146 | |
| Exon 7 | Forward | GGTCTTGCTCATGGCCTAAC | 147 | |
| | Reverse | AAGAGGAAATGCCACAAGGA | 148 | |
| Exon 8 | Forward | GGAAGGGATTGATTGTAGACCT | 149 | |
| | Reverse | AGGGGGAAGGAACAGCTAAG | 150 | |
| Exon 9 | Forward | TGAAGTTTTTGTCAGGGAACC | 151 | |
| | Reverse | CGCATAGAATTCATAACTTCCTC | 152 | |
| Exon 10 | Forward | CTGGGTTGGTAGTTGGGAAT | 153 | |
| | Reverse | CAACACCTGCAGTCCTCAAG | 154 | |
| Intron 11 | Forward | GCCCAGTCTGCAATTCTTCT | 155 | |
| | Reverse | GATCAGGCTTTGCACACAGA | 156 | |
| Exon 11 | Forward | TTGTCCTAAATGCCCCATGT | 157 | |
| | Reverse | CCTGGGCAGTGATGAAGG | 158 | |
| Exon 12 | Forward | CTCCATGCCACAGCAGACT | 159 | Intronic SNP |
| | Reverse | TCAGCTTGCCAAGAGAGTAAA | 160 | 4 bp insertion- |
| | | | | deletion |
| Exon 13 | Forward | AGACAGAAACGCCAATCCAG | 161 | |
| | Reverse | CAAGTGAACTGGTCGTGGTG | 162 | |
| Exon 13 | Forward | CAGCAGCTCCAGCCACAG | 163 | |
| | Reverse | TGCTTGTGAACAAGACAAACAG | 164 | |
| Exon 14 | Forward | AGCTTGTTTGGACCACATGA | 165 | |
| | Reverse | AGGCAGGGAGGACACTCAC | 166 | |
| Exon 15 | Forward | CAGCCCCTGGTGGTAGTAAA | 167 | |
| | Reverse | ACTTGAGGACTTGGCTGTGG | 168 | |
| Exon 16 | Forward | TCACCTGCCTCTTGACCTTT | 169 | |
| | Reverse | CCAACTCCCTCTGCTGGTC | 170 | |
| Exon 17 | Forward | GAGCCGAGAGGATGAAGATG | 171 | |
| | Reverse | GCTGCCCCTAACACTATGGA | 172 | |
| Exon 18, | Forward | TGGCAGCTACAATTGACATGA | 173 | 3′ UTR SNP |
| 3′-UTR | Reverse | CTGCTCCAGTCCACACAGG | 174 | |
| 3′-UTR | Forward | ACGTTTGTGACGTCCTACCC | 175 | |
| | Reverse | GCCACAGTCACACACTACCC | 176 | |
| 3′-UTR | Forward | CTCTTCTCCTCAGACACAGTGG | 177 | |
| | Reverse | GGGGCTCCAATTTAAAAACA | 178 | |
| 3′-UTR | Forward | GAAAGGGAGAGCCTGAGGAG | 179 | |
| | Reverse | CCAGGCCAGGGAGTTACA | 180 | |

PCR products are generated and haplotype variation of murine Brd4 is, in fact, observed between AKR and DBA tumor cells with SNPs in the regions described in Table 12. All the polymorphisms listed in Table 12 were observed in the AKR/J strain.

The identification of human SNPs in the Brd4 gene is explored. Specifically, published SNPs within human Brd4 are searched for using the dbSNP database of the NCBI website. Multiple SNP entries are found for Brd4. Four are characterized (Table 13). Brd4 polymorphisms are characterized in the constitutional DNA derived from lymphocytes from breast cancer patients using SNP-specific PCR. SNP-specific assays for fluorogenic PCR allelic discrimination (Assays-On-Demand®) are purchased from Applied Biosystems (Foster City, Calif.). The identities of the BRD4 SNPs characterized and the associated assay IDs are shown in Table 13.

TABLE 13

| | Position on | Location | | Applied |
|---|---|---|---|---|
| | Chr. 19 | Within | | Biosystems Assay |
| dbSNP ID | (bp) | BRD4 | Alleles | ID |
| rs4808272 | 15213372 | Intron 13 | A/G | C_2577207_10 |
| rs11880801 | 15224052 | Intron 10 | G/T | C_2577213_20 |
| rs8104223 | 15224477 | Intron 10 | A/G | C_29032171_10 |
| rs4809130 | 15248928 | 5′UTR | C/T | C_27942834_10 |

SNP-specific PCR using the assay are carried out as essentially described in Example 7 with the only difference being that primers and fluorogenic probes are replaced by the Applied Biosystems Assays-On-Demand® 20× assay mix. Statistical analyses of the data are carried out as essentially described in Example 7.

SNP frequencies are analyzed in the same cohort described in Example 7 (cases with localized disease [N=146] and cases with regional/metastatic disease [N=154]). The frequencies of each of the four characterized BRD4 SNPs are analyzed with respect to the same disease features described in Table 9 (stage of the disease, ER status, PR status, tumor size, grade of the tumor, presence of positive nodes, age at diagnosis, ductal histology, and lobular histology). SNP frequency analyses are performed for each of these characteristics for dominant and recessive models. All P values are based on Fisher's exact tests. This analysis shows a statistical significant association, between progesterone status (PR) of the tumor and rs11880801, since the TT among PR negative tumors is 14.3% compared to 2.6% among PR positive tumors (P=0.002; Table 14).

TABLE 14

| | | PR Negative | | PR Positive | | | | |
|---|---|---|---|---|---|---|---|---|
| | | Tumors | | Tumors | | Fishers Exact P Value | | |
| SNP ID | Genotype | N | % | N | % | All | Dominant | Recessive |
| rs4808272 | AA | 27 | 33.80% | 46 | 27.70% | 0.594 | 0.372 | 0.519 |
| | GA | 37 | 46.30% | 80 | 48.20% | | | |
| | GG | 16 | 20.00% | 40 | 24.10% | | | |
| | Total | 80 | | 166 | | | | |
| rs11880801 | GG | 41 | 58.60% | 110 | 71.40% | 0.004 | 0.066 | 0.002 |
| | GT | 19 | 27.10% | 40 | 26.00% | | | |
| | TT | 10 | 14.30% | 4 | 2.60% | | | |
| | Total | 70 | | 154 | | | | |
| rs8104223 | AA | 41 | 51.90% | 74 | 44.30% | 0.546 | 0.277 | 1.000 |
| | GA | 30 | 38.00% | 75 | 44.90% | | | |
| | GG | 8 | 10.10% | 18 | 10.80% | | | |
| | Total | 79 | | 167 | | | | |
| rs4809130 | CC | 67 | 83.80% | 128 | 77.10% | 0.101 | 0.245 | 0.325 |
| | CT | 12 | 15.00% | 38 | 22.90% | | | |
| | TT | 1 | 1.30% | 0 | 0.00% | | | |
| | Total | 80 | | 166 | | | | |

The foregoing demonstrates that a SNP in the BRD4 gene correlates with a more aggressive form breast cancer. Specifically, carriers of the rs11880801 variant allele appear more likely to have primary tumors lacking progesterone receptors, which is a hallmark of poor prognosis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A pharmaceutical composition comprising (i) a nucleic acid comprising a nucleotide sequence encoding a protein, (ii) a vector comprising the nucleic acid, (iii) a host cell comprising the vector, (iv) a gene product, or (v) a combination thereof, wherein the protein or gene product are encoded by a gene selected from the group consisting of: Anakin, Necdin, and Brd4, and a pharmaceutically acceptable carrier.

3. The pharmaceutical composition of claim 2, wherein the nucleic acid comprises the nucleotide sequence of SEQ ID NO: 2 or 4.

4. The pharmaceutical composition of claim 2, wherein the gene is an Anakin gene and the gene product is an Anakin protein or an Anakin mRNA.

5. The pharmaceutical composition of claim 4, wherein the Anakin protein comprises the amino acid sequence of SEQ ID NO: 1 or 3.

6. The pharmaceutical composition of claim 2, wherein the Necdin gene comprises the nucleotide sequence of SEQ ID NO: 10, or the Necdin gene product comprises the amino acid sequence of SEQ ID NO: 9.

7. The pharmaceutical composition of claim 2, wherein the Brd4 gene comprises the nucleotide sequence of SEQ ID NO: 108 or 110, or the Brd4 gene product comprises the amino acid sequence of SEQ ID NO: 109 or 111.

8. A method of preventing or inhibiting metastasis of a cancer cell in a subject comprising administering to the subject the pharmaceutical composition of claim 2 in an amount that is effective to prevent or inhibit metastasis of the cancer cell in the subject.

9. A pharmaceutical composition comprising (i) a nucleic acid comprising a nucleotide sequence encoding a protein, (ii) a vector comprising the nucleic acid, (iii) a host cell comprising the vector, (iv) a gene product, or (v) a combination thereof, wherein the protein or gene product are encoded by a gene selected from the group consisting of: CentaurinD3 (CentD3), Csf1r, Pi16, and Luc7l, and a pharmaceutically acceptable carrier.

10. A method of preventing or inhibiting tumor growth in a subject comprising administering to the subject the pharmaceutical composition of claim 2 in an amount that is effective to prevent or inhibit tumor growth in the subject.

11. A method of characterizing a tumor or a cancer in a subject comprising detecting (i) a single nucleotide polymorphism (SNP) in an Anakin gene or a Brd4 gene of the subject, (ii) an amino acid substitution in an Anakin protein in the subject, or (iii) an expression level of an Anakin gene or a Brd4 gene in the subject, whereupon the tumor or cancer is characterized.

12. The method of claim 11, wherein the tumor or cancer is characterized in terms of metastatic capacity, stage, tumor grade, nodal involvement, regional metastasis, distant metastasis, sex hormone receptor status, or tumor size.

13. The method of claim 12, wherein the sex hormone receptor is the estrogen receptor or the progesterone receptor.

14. The method of claim 11, wherein the SNP is located within an exon of an Anakin gene and results in an amino acid substitution.

15. The method of claim 14 wherein the amino acid substitution is a Leu substituted for a Pro at position 436 of SEQ ID NO: 1.

16. The method of claim 11, wherein the SNP is a T→C at position 1421 of SEQ ID NO: 2.

17. The method of claim 11, wherein the SNP is located within an intron of the Brd4 gene.

18. The method of claim 17, wherein the SNP is an A→G at position 14290 of SEQ ID NO: 112, a G→A SNP at position 3185 of SEQ ID NO: 112, or a G→T SNP at position 13865 of SEQ ID NO: 112.

19. The method of claim 11, wherein the subject is a mammal.

20. The method of claim 19, wherein the mammal is a human.

21. The method of claim 11, wherein the cancer is an epithelial cancer.

22. The method of claim 21, wherein the epithelial cancer is breast cancer.

23. The method of claim 21, wherein the epithelial cancer is renal cell carcinoma.

24. The method of claim 11, wherein detecting a SNP comprises detecting a complementary SNP.

25. The method of claim 11, wherein detecting a SNP comprises a polymerase chain reaction (PCR).

26. The method of claim 25, wherein the PCR is carried out using primers and probes comprising the nucleotide sequences of SEQ ID NOs: 5 to 8.

27. The method of claim 11, wherein the method is performed in vitro.

28. The method of claim 11, wherein the method further comprises comparing (i) the nucleotide sequence of the Anakin gene or the Brd4 gene of the subject, (ii) the amino acid sequence of the Anakin protein of the subject, or (iii) the expression level of the Anakin gene or the Brd4 gene in the subject to a control.

29. An isolated, purified, or synthetic nucleic acid comprising a nucleotide sequence selected from the group consisting of SEQ ID NOs: 5 to 8.

30. An isolated, purified, or synthetic antibody, or antigen binding portion thereof, which specifically binds to a murine Anakin protein or an Anakin allelic variant.

31. The isolated, purified, or synthetic antibody, or antigen binding portion thereof, of claim 30, wherein the murine Anakin protein comprises the amino acid sequence of SEQ ID NO: 3.

32. The isolated, purified, or synthetic antibody, or antigen binding portion thereof, of claim 30, wherein the Anakin allelic variant comprises the amino acid sequence of SEQ ID NO: 1 with an amino acid substitution of Leu to Pro at position 436 of SEQ ID NO: 1.

33. The isolated, purified, or synthetic antibody, or antigen binding portion thereof, of claim 30, wherein the antibody, or antigen binding portion thereof, specifically binds to an epitope comprising Pro at position 436 of SEQ ID NO: 1 or Leu at position 436 of SEQ ID NO: 1.

34. A kit comprising the antibody, or antigen binding portion thereof, of claim 30, or a nucleic acid which specifically hybridizes to a portion of a nucleic acid comprising a nucleotide sequence encoding an Anakin protein or Anakin allelic variant, or a combination thereof, and a set of user instructions.

35. The kit of claim 34, wherein the nucleic acid comprising a nucleotide sequence encoding an Anakin protein comprises the nucleotide sequence of SEQ ID NO: 2 or 4.

36. The kit of claim 34, wherein the nucleic acid comprising a nucleotide sequence encoding an Anakin allelic variant comprises the nucleotide sequence of SEQ ID NO: 2 with a T→C single nucleotide polymorphism (SNP) at position 1421 of SEQ ID NO: 2.

37. The kit of claim 34, comprising one or more of the nucleic acids comprising a nucleotide sequence selected from the group consisting of SEQ ID NOs: 5 to 8.

38. A method for screening a compound for anti-cancer activity comprising (a) providing a cell that (i) under-expression a nucleic acid comprising a nucleotide sequence encoding an Anakin protein or a Brd4 protein or (ii) comprises an Anakin or Brd4 allelic variant, (b) contacting the cell with a compound of interest, and (c) assaying for anti-cancer activity.

39. (canceled)

40. A method of inhibiting Sipa-1 in a subject in need thereof comprising administering to the subject an effective amount of (i) a nucleic acid comprising a nucleotide sequence encoding an Anakin protein, (ii) a vector comprising the nucleic acid, (iii) a host cell comprising the vector, (iv) an Anakin gene product, or (v) a combination thereof.

41. The method of claim 40, wherein the nucleic acid comprises the nucleotide sequence of SEQ ID NO: 2 or 4.

42. The method of claim 40, wherein the Anakin gene product is a protein or an mRNA.

43. The method of claim 42, wherein the protein comprises the amino acid sequence of SEQ ID NO: 1 or 3.

44. The method of claim 40, wherein the method effectively inhibits Sipa-1 GTPase activity.


SEQUENCE LISTING (180 sequences)

SEQ ID NO: 1 - protein, 758 aa, Homo sapiens
  MISC_FEATURE: Amino Acid Sequence of GenBank Accession No. NP_055871
MAPAMQPAEIQFAQRLASSEKGIRDRAVKKLRQYISVKTQRETGGFSQEELLKIWKGLFYCMWVQDEPLLQEELANTIAQLVHAVNNSAAQHLFIQTFWQTMNREWKGIDRLRLDKYYMLIRLVLRQSFEVLKRNGWEESRIKVFLDVLMKEVLCPESQSPNGVRFHFIDIYLDELSKVGGKELLADQNLKFIDPFCKIAAKTKDHTLVQTIARGVFEAIVDQSPFVPEETMEEQKTKVGDGDLSAEEIPENEVSLRRAVSKKKTALGKNHSRKDGLSDERGRDDCGTFEDTGPLLQFDYKAVADRLLEMTSRKNTPHFNRKRLSKLIKKFQDLSEGSSISQLSFAEDISADEDDQILSQGKHKKKGNKLLEKTNLEKEKGSRVFCVEEEDSESSLQKRRRKKKKKHHLQPENPGPGGAAPSLEQNRGREPEASGLKALKARVAEPGAEATSSTGEESGSEHPPAVPMHNKRKRPRKKSPRAHREMLESAVLPPEDMSQSGPSGSHPQGPRGSPTGGAQLLKRKRKLGVVPVNGSGLSTPAWPPLQQEGPPTGPAEGANSHTTLPQRRRLQKKKAGPGSLELCGLPSQKTASLKKRKKMRVMSNLVEHNGVLESEAGQPQALGSSGTCSSLKKQKLRAESDFVKFDTPFLPKPLFFRRAKSSTATHPPGPAVQLNKTPSSSKKVTFGLNRNMTAEFKKTDKSILVSPTGPSRVAFDPEQKPLHGVLKTPTSSPASSPLVAKKPLTTTPRRRPRAMDFF

SEQ ID NO: 2 - DNA, 5087 bp, Homo sapiens
  misc_feature: Nucleotide Sequence of GenBank Accession No. NM_015056
cgccgccttctgtgcagtcgcggcccgggcggacggtggctggctgctccgcagcgctcggctggctgcagcggcaccgcgggttgcgcggccggggatgctccagcgggcgcgatggcccccgccatgcagccggccgagatccaatttgcccagcggctggcgtccagcgagaagggcatccgggaccgagcggtgaagaagctgcgccagtacatcagcgtgaagacgcagagggagacaggaggtttcagtcaggaagaacttctgaaaatctggaaggggctcttctactgcatgtgggtgcaggatgaaccccttctacaggaagagctcgccaacaccattgcacagctagtccatgctgttaacaactcagcggctcaacacctgttcattcagaccttttggcaaaccatgaatcgagaatggaaaggaatagacaggctacgcctggacaaatactatatgctgattcgtctggtcctgaggcagtcctttgaagtcttgaagcgaaatggctgggaagaaagccgaatcaaggttttcttggatgtcctgatgaaggaggtcctgtgtcctgagagtcagtctcctaatggagtgagattccacttcattgatatttacctggatgaactctccaaagtcggggggaaggagcttttagcagatcagaatctcaagtttatcgatccattctgcaaaattgctgcgaagacgaaggaccacaccctggtacagaccatagctcggggtgtcttcgaagctatcgtagatcagtctccttttgtgcctgaagagacgatggaggaacagaagacaaaagtgggtgatggtgacctctctgctgaggagatacctgaaaatgaggtatccttgagaagagctgtcagtaaaaagaagacagcactgggcaaaaaccattccagaaaagatggactcagtgatgaaagaggaagagatgactgtggaacctttgaggacacagggccccttctccagtttgactataaggctgttgctgatcgactcctggaaatgaccagcaggaagaacacgccccacttcaacaggaagcgcctctccaaactcatcaagaaattccaagacctttctgaaggaagcagtatatctcaactcagttttgcggaggacatttctgctgatgaagatgaccaaatcctcagtcaaggaaagcataagaagaaaggaaataaacttttagagaaaactaacttggaaaaggagaaaggaagcagagtcttttgtgtagaggaagaggacagtgaaagcagtcttcaaaagagaagaaggaagaagaagaagaagcaccacctgcagcctgaaaatccaggcccagggggtgcagccccgtccctggaacagaaccggggcagggagcccgaggcctctgggctgaaagccctgaaggcacgtgtggccgagccaggtgcagaggccacgtccagcactggggaggagagtggctccgagcatcctccagccgtccccatgcacaataaaaggaaacggccacggaagaagagcccgagggcccacagggaaatgttggaatcagcagtgttgcccccagaggacatgtctcagagtggcccgagtggcagtcatcctcagggacctagagggtccccgacaggtggagcccaactcctaaaaaggaagcggaaacttggagttgtgcccgtcaatggcagtggcctgtccacgccggcctggcctccattgcagcaggaaggccctcccacaggccccgcagagggggcgaacagccacaccacgctgccccagcgcaggaggctgcagaaaaagaaggcagggcccggcagcctggagctctgtggcctgcccagccagaaaacagcaagtttgaaaaagaggaagaaaatgagagtgatgtcaaacttggtggagcacaacggggtgctggagtccgaagctgggcaaccccaggctctgggaagcagtgggacttgcagttccctgaagaagcagaagctgagggcagagagcgactttgtgaagtttgacacccccttcttaccaaagcccctgttcttcagaagagccaagagcagcactgccacccaccctccaggccctgccgtccagctaaacaagacaccatccagctccaagaaagtcacctttgggctgaacagaaacatgactgccgaattcaagaagacagacaagagtatcttggtcagtcccacgggcccttctcgagtggccttcgaccctgaacagaagcccctccacggggtgctgaagacccccaccagctcacctgccagctcacccctggtggccaagaagcccctgaccaccacaccaaggagaaggcccagggctatggatttcttctgaggagcagcagagtcccttgtaaaagactgcttttgtacagaatgcgctataaattatacctttaagaatgtggggccttttttatgattttgtaagttcccataagttgtgtgcacgaggttctgagagtgcccgcaggctgctgcgtcctggcccctctgtagtggctgcgggcgtcttggttgaatcttttgctacaaaccatgtttgcgtttgagctctccaggattttacatttttgggtaacctcagtgattcccattggtgtaggaaatgagaccctctctgaagctgaggagagcacgttgatctgaactttaaatcaatcagtgctgctggcacaatgaaaggtggaactgcacttctgttgagctctcagttctgcggaatttggtactcattaccgtattcgccgtactaagttggtttctgttagtcttaacagtctgttttcttttaaaagcatgtagggcttcattgccatgttctgtgggtgtttggcaggttaccgatggggaagattcttgtcacagaatcagcaataccatagtttttctacatgtgctcagctgggggtgtggacaggtaggggtggggaaagaagaggctctgcgttctgggggctttttcttctcctccccctacccggtttccctccctgttttcctacctctacggcaagcccaaagtgtcttcccgggagcccagcgcagcccccggctcttacccaggaccccgccccgtgctgagccttctgctgaggtccttgcgtggagcacactcattcctccaagcccttgcgctcccgtttctctctctctccgtccacgttccagccgagtcactgcctgaccggctccatggcagctccccatcttccctagaggctgcctgcgcatctggagcctgcgctccggctcagcgacctttcctctcaaatgcggaagcgtgcacttacagttcagaccgttctcctgtaagttcattacaaacacgggcggaaggcactcaggctttcgttggagaaacagaaataaggccttcttttgagcagcgattgctggatcattgatctgtttgaggaagtgtctgacctgggcctgagagctggagaaggtgcagattcaaagtgagcggctcctgaggagagccgccaaggctgctcgccttctccgtggcttccgcagctaccgtctgcacggtgagagggcacgggcacacggttcgggctggcgtgcagctctcccagccagccacgctctgctcaggcctggaagtgaaagccgcctccttcccgttatgccccccatacaggagcctcggtttttcagcaaaacgcggccagtccccttctccactgctgcctcccagcagagggccccaggatctccaaggtcccagctatggctttggacaacgtggcttcggcccctggggttgcagagcttgcattgggtttacctcggtctcattcattcatggagccaagggtggggtttcacctgcgaacatcagactgacttgctggcgtcaagagcagttgactcactgatgaaggccctggtgaggagaaagcactctgttcttcgcctactctgtaatcgttttgtcataatgagccatgaaaaaagtaatgaacttgtgctgttaatcgtcactgtaatgagaagtcttacgtacaacatagctgtggtggcttaatggctgcattagataggatcctcacatcccattcagaaccaaaactgatacagtgaaacaattaaggtgagcaaatagttttaacttttcttttttttttttaagtttcattcttcctagaatatttttctaacaatttttatttcagctttaaagatgggtcatatagccaaacgggccatataatccaacattgttgagatgtcttaggacatctaaggcaaaactggcacatttgttctgcagactattgcaggaatgttttttcctagcatttctatattatctgtccattctgaggaaccagtgaatgtcctataaatgcacctcctgtcaaaaccatgcctgagaggtcccggctgggagtgacagggtgcttcttagattctattggtccttctctcattctccgaacttactcctttttatgggtaagtcaactaggtttacagtcccttatttttaatgcctaagttttgacagcaggaagaaaacaattttttaaaaattctcattacatagacgcacaagaatatgtcacataaagaaaatgtgtttagaatactggttttctatttacgcatgatattttcctaagtaaaattgccaagtggacttggaagtccagaaaggaaaataatttaaattaatgctggtgatcttaacaatattttgtaaaatgatgcttcccccttctccatggtctagtcaattttgtacaattaggtatctgactttacaagtttgttatcctttctaatttttactgaactgaaagcacaaagaagactacacagaaaatctggaaacagttgcaggtgttgggaggaagatgaaatcgagctgtcttttaacttttgtatgtgttttatcagaatttgctggactatgctggcaaggactttgtttacgatcaaattgtactagtgtctgcagggtttgtcagtactcgtcaaagccaagtccaattaaaaaaaaaagtctttgccctccaa

SEQ ID NO: 3 - protein, 724 aa, Mus musculus
  MISC_FEATURE: Amino Acid Sequence of GenBank Accession No. NP_082520
MALAMQSSEFQFAQRLASSEKGVRDRAVRKLRQYLSARTQSDTGSFSQEELLKIWKGLFYCMWVQDEPLLQEELANIISQLIHVVNSLEAQYLFIQTFWQTMNREWQGIDKLQLDKYYMLIRLVLRQSFEVLKRNAWEESQITLFLDILMKEILSPESQSPNGVRTHLIDVYLEELTTVGGAELLADQNLKLIDPFCRIAAKTKDHTLAQTVARGVFEVIVDQSACVPEESVEERKTKEDGSGFPTKALACRKAVSGKKAALDECLRDGVIGSRERDICAALKDSGSPLQFDYKAVADRLLEIANSKSTPPFNRKRLCRLVRKFQDLCEGNGAPLSSAEDNGQRRHKRKRKKLLESEKGDTVSPAAEEDSGGHIHKKKRKKRKRSHFQPDTQNLDAVAVPKVPDSESEPDTAQRQAPCGQACVTEPTAEAVSSIGENSSKPTPVMPIHNKRKRPRKKKLRAHKEICKSTTLPQEDMSKNDAVSGHSQSSAAHISSSEGVQAQKRKRKLGALPDSSSDLPVQKSGTPTSPVEGKDGQTTLPRCKRSQKKTASSTLDPCDPSSQKPAISKKKKKTMKLMSNGVLESNPGQIQALGSNRTLKKPLKTEDDFVKFDTRFLPKPLFFRKAKNSSATRPQGPAGQLNKTPSSSKKVTFGLNRNMTAEFKKTDKSILVSPTGLSRVAFNPEQRPLHGVLKTATSSPASTPLSPMRLPATTPKRRPRAADFF

SEQ ID NO: 4 - DNA, 2564 bp, Mus musculus
  misc_feature: Nucleotide Sequence of GenBank Accession No. NM_028244
gatagacctttgttagctgtgcttagactctaaaactgcatcaaaccagccgtaggcgttctgctggaggagacctgacccccaactaaccgccctgtcaatcgccctgaacctgggccactctcaggacgcacttcaagcagcgttgctacggcgtctacccgcacaggggccacgcgggctccctgggcgcgcggcgagcggcgcaccggtgacgccatcacccgcgcgccgcagcccatacgcagacgcagtccggatcgcttggcggcgagcggcacgcgcgcagcctggggatgctgaagcgagtgcgatggctctcgccatgcagtcttccgagttccagttcgcccagcggctggcctccagtgagaagggcgtccgcgaccgcgccgtgaggaagctgcggcagtatctcagcgccaggacgcagagcgacacaggaagtttcagtcaagaagaactcttaaaaatatggaaaggactcttctactgcatgtgggtgcaggacgagccccttctgcaggaggagctagcgaacattatttcccaactcatccacgttgtaaacagcttggaggctcagtacctgtttattcagaccttctggcagaccatgaatcgagagtggcaagggatagacaagctgcagctggacaagtactacatgctgatccgcctggtcctgaggcagtcctttgaagttctcaagcggaatgcctgggaagaaagccaaatcacactgtttttggacatcctgatgaaggagatcctgagtcctgagagtcagtctcccaatggcgtgagaacccacttgattgatgtgtacttggaggagcttaccacagtgggaggggcggagctcttagcggaccagaacctcaagttaatcgatccattctgcagaattgctgccaagactaaggaccacacgctggcacagactgtagcgaggggtgtttttgaagtcattgtagatcagtctgcctgtgtacctgaagagtctgtggaggagcggaaaaccaaagaggatggcagtggcttccctacaaaggcgttggcctgtaggaaggcagtcagtgggaagaaggctgcactagacgaatgcctcagagacggagtcattggcagcagagaaagagacatctgtgcagccttgaaagattcagggtcacctctccagtttgactataaggctgttgctgatcgactcctggaaatagccaactcaaaaagcaccccgcctttcaacaggaagcgactctgcagactagtcagaaagttccaggacctctgtgaaggcaatggtgctccactcagttctgctgaggacaacggtcagagaaggcacaagaggaaaagaaagaagcttttagagagtgagaaaggagacacggtctctcctgctgctgaagaggacagcggaggccacattcacaagaagaaaaggaaaaagaggaagaggagtcacttccagcctgacactcagaacctggacgctgttgccgtgcccaaagtaccggattcggagagtgagcctgacactgcccagaggcaggccccgtgtgggcaagcgtgtgtgactgagcctacagcagaggctgtgtccagcatcggggagaacagctccaagcctacacccgtcatgcccatacacaataaaagaaaacggccgagaaagaagaaactgagggcccacaaagagatctgcaaatccaccactttacctcaggaggacatgtcaaagaatgacgctgtcagtgggcattctcagagctctgctgcccacatttcttcctcagaaggtgtccaagcccagaaaaggaaacggaaactgggagctctccctgacagcagtagtgacctacctgtacagaagtcagggaccccaacaagcccagtggaggggaaggatggccagaccaccctgccccggtgtaagaggtcacaaaagaagacagcatccagcacccttgacccctgtgatccgtccagtcaaaaaccagcaatctcaaaaaagaagaagaaaactatgaagctgatgtcaaacggtgtgttggagtccaaccctgggcagatccaagctctgggaagcaacaggactctgaagaagccgctgaaaacggaggacgactttgtgaagtttgacactcggttcttaccaaagcccctgttcttcaggaaagccaagaacagctctgccacccgtccccaaggtcctgctggccagctgaataaaacaccctccagttccaagaaagtcacctttggattgaacagaaacatgactgcagaatttaagaagacagacaagagtatcctggtcagccccacaggcctctccagagtggcctttaaccctgagcagaggccactccacggagtgctgaagaccgccacgagctccccggccagcactcctctgtcgcccatgaggctaccagccaccaccccaaagagaaggccaagggctgcggacttcttctgagagccagaagccctttctgaagcctgcttttataccggatgttctataaattataacttttaaaaaaaaaaaaaaa

SEQ ID NO: 5 - DNA, 18 bp, Artificial
  Synthetic
tggacgtggcctctgcac

SEQ ID NO: 6 - DNA, 19 bp, Artificial
  Synthetic
caccacctgcagcctgaaa

SEQ ID NO: 7 - DNA, 18 bp, Artificial
  Synthetic
agggctttcagcccagag

SEQ ID NO: 8 - DNA, 16 bp, Artificial
  Synthetic
agggctttcggcccag

SEQ ID NO: 9 - protein, 321 aa, Homo sapiens
  MISC_FEATURE: Genbank Accession No. NP_002478 for SEQ ID NO. 9.
MSEQSKDLSDPNFAAEAPNSEVHSSPGVSEGVPPSATLAEPQSPPLGPTAAPQAAPPPQAPNDEGDPKALQQAAEEGRAHQAPSAAQPGPAPPAPAQLVQKAHELMWYVLVKDQKKMIIWFPDMVKDVIGSYKKWCRSILRRTSLILARVFGLHLRLTSLHTMEFALVKALEPEELDRVALSNRMPMTGLLLMILSLIYVKGRGARESAVWNVLRILGLRPWKKHSTFGDVRKLITEEFVQMNYLKYQRVPYVEPPEYEFFWGSRASREITKMQIMEFLARVFKKDPQAWPSRYREALEEARALREANPTAHYPRSSVSED

SEQ ID NO: 10 - DNA, 1897 bp, Homo sapiens
  misc_feature: Genbank Accession No. NM_002487 for SEQ ID NO. 10.
acttcctctccaggaatccgcggagggagcgcaggctcgaagagctcctggacgcagaggccctgcccttgccagacggcgcagacatgtcagaacaaagtaaggatctgagcgaccctaactttgcagccgaggcccccaactccgaggtgcacagcagccctggggtttcggagggggttcctccgtccgcgaccctggcagagccgcagagccctcctctaggcccgacggccgctccgcaggccgcgccgcctccccaggccccgaacgacgagggcgacccgaaggccctgcagcaggctgcggaggagggccgcgcccaccaggccccgagcgcggcccagccgggcccggcaccgccagccccggcgcagctggtgcagaaggcgcacgagctcatgtggtacgtgctggtcaaggaccagaagaagatgatcatctggtttccagacatggtgaaagatgtcatcggcagctacaagaagtggtgcaggagcatcctccggcgcaccagcctcatcctcgcccgggtgttcgggctgcacctgaggctaaccagcctgcacaccatggagtttgcgctggtcaaagcgctggagcccgaggagctggacagggtggcgctgagcaaccgcatgcccatgacaggcctcctgctcatgatcctgagcctcatctacgtgaagggccgcggcgccagagagagcgccgtctggaacgtgctgcgcatcctggggctgcggccctggaagaagcactccaccttcggggacgtgcggaagctcatcactgaggagttcgtccaaatgaattacctgaagtaccagcgcgtcccatacgtggagccgcccgaatacgagttcttttggggctcccgggccagccgcgaaatcaccaagatgcaaatcatggagttcctggccagggtctttaagaaagacccccaggcctggccctcccgatacagagaagctctggaggaggccagagctctgcgggaggctaatcccactgcccactaccctcgcagcagtgtctctgaggactagcaaagtctggaggcagatgaatggtttctgaccctcaccagggctgtggaagggtgggggtgggtcattatagtattcaggatttacagtgcagtattcacgtgtaacttttaagttttcagtacagtgcttttatacctttaatgcaatgttgtattcatttgggtactattgtgtagtatttaggatgtatgcatgtttgtttatatgtaagcttggttggtgctttcgcttttgtgctacctttcttggatttttgtaccagagatgtgctaaactgatgaaatacattgagaaagtttccatcttattcttttatatgggactgatgatgtgtgttggggtagactgctcctgcagagtttggaagaagtcaccagcaaagccggcctaaccaagaaaagtcaaggcccttcatgaccttgctgggcacagaaaacaccctcgtggagtacactaatttgaactggactggtctcagtgtgagcacttggcacactttactaaacacatatacaaccccaccgtgagtcaactttaaagtaaacattaaagattcttgtgatacaatcatttttggaaaagtgtactttatcattttaacaaagcagtatggttgggaatgagacaattctctattttacagtgtatacagatacaactatttcccctaatagggtgggaaaaatcgctactcatgattactcctaaatttgtgaagtttatagttctattgtctttaaatgtaactcatgtttatttcaaaaacattcacaaatatagaaaagtatacaaaacaaaacagtaagattgtctgtaatcacatcatatgggaataaaaaaca

SEQ ID NO: 11 - protein, 325 aa, Mus musculus
  MISC_FEATURE: Genbank Accession No. NP_035012.2 for SEQ ID NO. 11.
MSEQSKDLSDPNFAAEVPDCEMQDSDAVPVGIPPPASLAANLAGPPCAPEGPMAAQQASPPPEERIEDVDPKILQQAAEEGRAHQPQSPARPIPAPPAPAQLVQKAHELMWYVLVKDQKRMVLWFPDMVKEVMGSYKKWCRSILRRTSVILARVFGLHLRLTNLHTMEFALVKALSPEELDRVALNNRMPMTGLLLMILSLIYVKGRGAREGAVWNVLRILGLRPWKKHSTFGDVRKIITEEFVQQNYLKYQRVPHIEPPEYEFFWGSRANREITKMQIMEFLARVFKKDPQAWPSRYREALEQARALREANLAAQAPRSSVSED

SEQ ID NO: 12 - DNA, 1765 bp, Mus musculus
  misc_feature: Genbank Accession No. NM_010882 for SEQ ID NO. 12.
cggacgcgtgggcgctccaagagctccaagccgcatcggtcctgctctgatccgaaggcgcagacatgtcggaacaaagtaaggacctgagcgaccctaactttgcagccgaggtccccgactgtgagatgcaggacagcgatgccgttccggtggggatccctcctcccgcttctctggccgctaacctcgcagggccaccgtgcgctcccgaaggccctatggcagcccaacaggcctcgccaccgcccgaagaacggatagaagatgttgaccctaaaatcctgcagcaggccgcagaggagggccgcgcccaccagccccagagtccagcccggccgatcccagcaccgccagcccctgcccagctggtgcagaaggcgcacgagctcatgtggtacgtgttggtgaaggaccagaagaggatggtcctctggtttccagacatggtgaaagaggtcatgggcagctacaagaaatggtgcagaagcatcctcaggcgcaccagcgtcatcctcgccagagtgttcgggctgcacctgaggctgaccaatctccacaccatggagtttgccctggtcaaagccctcagcccagaggagctagacagggtggcgctcaacaaccgtatgcccatgacaggcctcctgctcatgatcctgagcctcatctatgtgaagggccgcggggccagagagggtgcggtctggaatgtgctgcgcatcctggggctgaggccctggaagaagcactccaccttcggagacgtgaggaagataatcaccgaggagttcgtccagcagaattacctgaagtaccagcgtgtgccccacatcgagcctcccgagtacgagttcttctgggggtccagagctaaccgtgaaatcaccaagatgcagatcatggagttcctggccagagtcttcaagaaagatccccaggcgtggccttcccgatacagggaggctctggagcaggccagagctctgcgggaggctaatcttgctgcccaggccccccgcagcagtgtctctgaggactaaaaaggtccaggggcacactgatagtttctgacccatactagggctgtgtaagggtggggttgagtcattagagtatcccaaatccacagtgcagtatttcatgtataatttttaagttttccatacagtgcttttgtaccttgtaatgctattcatttgtgtactcgtgtagtgtttaagattgatgcatgtgtgataagtatttggtactttcacttttgtgctttcgtgcatttttgtacaagagatgtgctgtgctaaacttgtgaaatacattgaggtgttctgtatcttgttctttgtatgggactgatgatctgtatcgacaaagaaggccctggagagttagcaggacttaacagcaacgcagacctgagcaagagaaaggtcaaggcctttctccatatgacttcaactggcacaggaagcatccatgtggaatggactgatttgaactggactgttctcagtgtaggcacttagcaccctttacaaaacatgtatgcaaccccaccataaataaacgttaaaatgagcattaaaaaaaaaaaaaaaaaaaaaaaagggcggccgctcgcgatctagaactagtcttaagcggggtgggagggcaagggagggtgccctcctagtggggtttggggggattgggttcctgaatgcaccataattgctgtatgaaatattaaaaaaaagtctaaagttcaaaaaaaaaaaaaaaa

SEQ ID NO: 13 - protein, 1544 aa, Homo sapiens
  MISC_FEATURE: Genbank Accession No. NP_071926 for SEQ ID NO. 13.
MAAPQDLDIAVWLATVHLEQYADTFRRHGLATAGAARGLGHEELKQLGISATGHRKRILRLLQTGTEEGSLDPKSDSAMEPSPSPAPQAQPPKPVPKPRTVFGGLSGPATTQRPGLSPALGGPGVSRSPEPSPRPPPLPTSSSEQSSALNTVEMMPNSIYFGLDSRGRAQAAQDKAPDSSQISAPTPALRPTTGTVHIMDPGCLYYGVQPVGTPGAPDRRESRGVCQGRAEHRLSRQDLEAREDAGYASLELPGDSTLLSPTLETEETSDDLISPYASFSFTADRLTPLLSGWLDKLSPQGNYVFQRRFVQFNGRSLMYFGSDKDPFPKGVIPLTAIEMTRSSKDNKFQVITGQRVFVFRTESEAQRDMWCSTLQSCLKEQRLLGHPRPPQPPRPLRTGMLELRGHKAKVFAALSPGELALYKSEQAFSLGIGICFIELQGCSVRETKSRSFDLLTPHRCFSFTAESGGARQSWAAALQEAVTETLSDYEVAEKIWSNRANRQCADCGSSRPDWAAVNLGVVICKQCAGQHRALGSGISKVQSLKLDTSVWSNEIVQLFIVLGNDRANRFWAGTLPPGEGLHPDATPGPRGEFISRKYRLGLFRKPHPQYPDHSQLLQALCAAVARPNLLKNMTQLLCVEAFEGEEPWFPPAPDGSCPGLLPSDPSPGVYNEVVVRATYSGFLYCSPVSNKAGPSPPRRGRDAPPRLWCVLGAALEMFASENSPEPLSLIQPQDIVCLGVSPPPTDPGDRFPFSFELILAGGRIQHFGTDGADSLEAWTSAVGKWFSPLSCHQLLGPGLLRLGRLWLRSPSHTAPAPGLWLSGFGLLRGDHLFLCSAPGPGPPAPEDMVHLRRLQEISVVSAADTPDKKEHLVLVETGRTLYLQGEGRLDFTAWNAAIGGAAGGGGTGLQEQQMSRGDIPIIVDACISFVTQHGLRLEGVYRKGGARARSLRLLAEFRRDARSVKLRPGEHFVEDVTDTLKRFFRELDDPVTSARLLPRWREAAELPQKNQRLEKYKDVIGCLPRVNRRTLATLIGHLYRVQKCAALNQMCTRNLALLFAPSVFQTDGRGEHEVRVLQELIDGYISVFDIDSDQVAQIDLEVSLITTWKDVQLSQAGDLIMEVYIEQQLPDNCVTLKVSPTLTAEELTNQVLEMRGTAAGMDLWVTFEIREHGELERPLHPKEKVLEQALQWCQLPEPCSASLLLKKVPLAQAGCLFTGIRRESPRVGLLRCREEPPRLLGSRFQERFFLLRGRCLLLLKEKKSSKPEREWPLEGAKVYLGIRKKLKPPTPWGFTLILEKMHLYLSCTDEDEMWDWTTSILKAQHDDQQPVVLRRHSSSDLARQKFGTMPLLPIRGDDSGATLLSANQTLRRLHNRRTLSMFFPMKSSQGSVEEQEELEEPVYEEPVYEEVGAFPELIQDTSTSFSTTREWTVKPENPLTSQKSLDQPFLSKSSTLGQEERPPEPPPGPPSKSSPQARGSLEEQLLQELSSLILRKGETTAGLGSPSQPSSPQSPSPTGLPTQTPGFPTQPPCTSSPPSSQPLT

SEQ ID NO: 14 - DNA, 5281 bp, Homo sapiens
  misc_feature: Genbank Accession No. NM_022481 for SEQ ID NO. 14.
gggacccagaacctcggacgagcggcgggcacccgcgagcggacggcggccgcgtagtgagcaatggcctgagcccccatggctgcccctcaggacctggacatcgctgtgtggctggccacggtgcacctggagcagtatgcagacacgttccgacggcatggcctggctacagcaggtgcagcccggggcctgggccacgaggagttgaagcagttgggcatcagcgccacagggcaccggaaacgcattctacgcctgctacagacaggcaccgaagagggctccctggatcccaaatcagatagtgccatggaaccatcccccagcccagccccgcaagcccagccccctaagcccgtgccgaagcccaggaccgtgtttggtggactcagtggccctgccaccactcagagacctgggctgagcccagccctcgggggaccaggagtgtccaggagcccagagcccagcccaaggcctcctcctctccccacttcctcctctgagcagtcttcagccctaaatactgtggagatgatgcctaattccatctacttcggcctggactcaagaggcagggcacaggcagctcaggacaaggccccagacagctcccaaatctctgcccccacccctgccctcaggcccacaacaggcacagtgcacatcatggatcctggttgcctgtactatggtgtccaacctgtggggactccaggagcccccgacagaagagagagcagaggtgtttgtcagggcagggctgaacacaggctcagcagacaggatctggaggcacgggaggatgctggctatgccagccttgagctacctggagactccaccctcttatcgcccaccctggaaacagaggagaccagtgatgacctcatttcaccctatgccagcttctccttcacggcagaccgcctcacgcccctgctcagtggctggctagacaagctctcccctcagggaaactatgtcttccagagacgctttgtgcagttcaatgggaggagtctgatgtactttggcagtgacaaggaccccttccctaagggtgtgatacctttgactgccattgagatgacccgcagcagcaaggacaacaagttccaggtcatcaccggccagagggtgttcgtgttccgcacagagagcgaggctcagcgggacatgtggtgctccacgctgcagtcctgtctgaaggagcagcgcctcctgggccacccccggcccccccaaccaccccgacccctccgcacgggcatgctggagctgcgtggacacaaggccaaggtgtttgctgccttgagccctggagagctggcactgtacaagagtgagcaggccttctctctgggcatcgggatctgcttcatcgaactgcagggctgcagcgtccgggagaccaagagtcgaagctttgacctgctcacaccccatcgctgcttcagcttcacagccgagtctgggggtgctcggcagagctgggcggccgctctgcaggaagcagtaaccgagaccctgtctgactacgaggtggctgagaagatctggtctaatcgggccaaccggcagtgtgcggactgtgggtcctcccgcccagattgggctgctgtcaatttgggggtggtcatctgcaagcagtgtgcaggtcagcaccgggccctgggttctgggatctccaaggtgcagagcctgaagctggacacgagtgtctggagtaatgagatagtacagttattcattgtcctgggaaatgatcgtgccaaccgcttctgggcagggaccctacccccaggtgagggactacatccagatgcgacccctggcccccggggagagttcatctcccgaaagtaccgtctgggtctcttccggaagccccaccctcagtacccagatcatagccagcttctccaggcactgtgtgcagctgtggcaagacccaacctgctgaagaacatgacccagctcctctgtgttgaggcctttgaaggcgaggagccctggttccccccagcccctgatggcagctgccctggcctcttgccctcagacccctcccctggtgtgtacaatgaggtggtggtgcgtgctacttacagcggcttcctgtactgcagtcccgtcagcaacaaagctggaccctcaccccctcgcaggggccgggatgctcccccgcgcctttggtgtgtgctgggagcagctctggaaatgtttgcatcggaaaacagccctgaacccctcagcctcatacagccccaggatattgtatgtctgggtgtgagccccccacccactgacccaggtgacaggttccccttttcctttgagctcatcctcgctggggggaggatccagcattttggcacagatggagctgacagtctggaggcctggactagtgctgtgggcaagtggttctccccgctgagctgccaccagctgctgggccccgggctgctgcggctgggccgcctatggctgcggtccccctcccatacagccccggcccctggtctctggctgtcagggtttggcctccttcgtggtgaccacctcttcctgtgctcagcgccgggcccaggccccccagcccctgaggacatggtgcatctgcggcggctacaggagatcagtgtggtttctgcagctgacaccccagataagaaagagcatttggtcctggtggagacaggaaggaccctgtatctgcaaggagagggccggctggacttcacggcatggaacgcagccattgggggcgcggctggtgggggcggcacagggctgcaggagcagcagatgagccggggtgacatccccatcatcgtggatgcctgcatcagttttgttacccagcatgggctccggctggaaggtgtataccggaaagggggcgctcgtgcccgcagcctgagactcctggctgagttccgtcgggatgcccggtcggtgaagctccgaccaggggagcactttgtggaggatgtcactgacacactcaaacgcttctttcgtgagctcgatgaccctgtgacctctgcacggttgctgcctcgctggagggaggctgctgagctgccccagaagaatcagcgcctggagaaatataaagatgtgattggctgcctgccgcgggtcaaccgccgcacactggccaccctcattgggcatctctatcgggtgcagaaatgtgcggctctaaaccagatgtgcacgcggaacttggctctgctgtttgcacccagcgtgttccagacggatgggcgaggggagcacgaggtgcgagtgctgcaagagctcattgatggctacatctctgtctttgatatcgattctgaccaggtagctcagattgacttggaggtcagtcttatcaccacctggaaggacgtgcagctgtctcaggctggagacctcatcatggaagtttatatagagcagcagctcccagacaactgtgtcaccctgaaggtgtccccaaccctgactgctgaggagctgactaaccaggtactggagatgcgggggacagcagctgggatggacttgtgggtgacttttgagattcgcgagcatggggagctggagcggccactgcatcccaaggaaaaggtcttagagcaggctttacaatggtgccagctcccagagccctgctcagcttccctgctcttgaaaaaagtccccctggcccaagctggctgcctcttcacaggtatccgacgtgagagcccacgggtggggctgttgcggtgtcgtgaggagccacctcgcttgctgggaagccgcttccaggagaggttctttctgctgcgtggccgctgcctgctgctgctcaaggagaagaaaagctctaaaccagaacgggagtggcctttggaaggtgccaaggtctacctgggaatccgcaagaagttaaagcccccaacaccgtggggcttcacattgatactagagaagatgcacctctacttgtcctgcactgacgaggatgaaatgtgggattggaccaccagcatccttaaagcccagcacgatgaccagcagccagtggtcttacgacgccattcctcctctgaccttgcccgtcagaagtttggcactatgcctttgctgcctatccgtggggatgacagtggagccaccctcctctctgccaatcagaccctgcggcgactacacaaccggaggaccctgtccatgttctttccaatgaagtcatcccaggggtctgtggaggagcaagaggagctggaggagcctgtgtacgaggagccagtgtatgaggaagtaggggccttccctgagttgatccaggacacttctacctccttctccaccacacgggagtggacagtgaagccagagaaccccctcaccagccagaagtcattggatcaaccctttctctccaagtcaagcacccttggccaggaggagaggccacctgagccccctccaggccccccttcaaagagcagtccccaggcacgggggtccctagaggaacagctgctccaggagctcagcagcctcatcctgaggaaaggagagaccactgcaggcctgggaagtccttcccagccatccagcccccaatcccccagccccactggccttccaacacagacacctggcttccccacccaacccccatgcacttccagtccaccctccagccagcccctcacatgaccctaggaccagcagtctgagagggtaggtaccagaagacccagaaactcttatcgtggcactgttgcagcttcctctgccctggctggaaagactccagaatccagtgtggtgctgtggaaggagcactggactaaaggcttcagtggctgcgtgtcccaggacaggtcatggcccctctctgggcccagcccatttatctataccatgaggtaactgaagtaaggagagcagtgaatgtcaaactgtgtttcttagagccataagccccacatattatccctgaacaagggcagctcctgctttatatatttgatacgtaggggttccatgagagattttgggttttaaaggaatggttttactgcattaaagaaaaaaaatgctttggaaaccagaggcctgggtgatgttaaagtctatcctgtcccacttcctacattctgggactaccgtgaagcctggagtagggagagcgagtttgggagctgggactcggggagtcaaaaatagatgagtaattgtcaataaacctgggaaccaaaagacaaaaaaaaaaaaaaaaaaa

SEQ ID NO: 15 - protein, 972 aa, Homo sapiens
  MISC_FEATURE: Genbank Accession No. NP_005202 for SEQ ID NO. 15.
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 16 - DNA, 3985 bp, Homo sapiens
  misc_feature: Genbank Accession No. NM_005211 for SEQ ID NO. 16.
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcctccaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 17 - protein, 722 aa, Homo sapiens
  MISC_FEATURE: Genbank Accession No. NP_055114 for SEQ ID NO. 17.
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETGPA

SEQ ID NO: 18 - DNA, 3149 bp, Homo sapiens
  misc_feature: Genbank Accession No. NM_014299 for SEQ ID NO. 18.
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaagaaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacaggtcctgcctaatcattggacacggactcttaataaaacggtcttcagttccagattccttcccagcaagctatagcttaagtccattttcttccgtgaaagggacaggactccatcaagttatggaattcctcagagccctgggcctgtcccccggggtggattagtcatgtccagcagcacacgcctagtcccgccttcgggaaggctgcctgcctggccagccgcccaggcctctctgtgtaaagactgcctggctgtcctgcccagccttcctggttctctggggtcctctgggtgggtggcatctcctggagggtgatgacaatccccaacacatgcattcatgtggtgctactctgtgtgcaaagccagaccccaagtatgttttctctctttgtcccatccctctttttctgggactttggaccctaactacttccctcctgaaccttgcagtgacatcagtccaggagagctctcgttcagtgtgcggaagaacactctgacctctagagctgtcctagataaggagtgggagctttagaggcaaggcctctagaccctggaaggctcagtgaggctcttcccacagcatgcttctcactggtgccctgtaagctcgagccaccgctgactctgagcctttttggagtctttcctccttcgtctccattgttccgtgcatttccaaagcttaagttgctggtgggcatttccccagtttctatgggctccgtcttctcaagtcacatagggaaagtaccttc

SEQ ID NO: 19 - protein, 436 aa, Homo sapiens
  MISC_FEATURE: Genbank Accession No. NP_699201 for SEQ ID NO. 19.
LTDEEKRLMVELHNLYRAQVSPTASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCEPIGSPEDAQDLPYLVTEAPSFRATEASDSRKMGTPSSLATGIPAFLVTEVSGSLATKALPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIF

SEQ ID NO: 20 - DNA, 2205 bp, Homo sapiens
  misc_feature: Genbank Accession No. NM_153370 for SEQ ID NO. 20.
cctgggtgcaaccagtcacagctctgcagaggttactgtgattttgcccctgaaggatctgtccacaacttaggaactcacacagcttttggcctgagcccccgttaccaagagaaaggaggtttttgccaaggactccaaggggagtgcacttgatgctggtcgggacccaaagcgcccagccctccctgagacattgtgtgagtcgggctgggcctcaaacacggcccccactgccccaccccagccagggtggtgcttgtgtgggaaggactttaaatccagctgccagacccctggacgggagaaggagagacggctggccaccatgcacggctcctgcagtttcctgatgcttctgctgccgctactgctactgctggtggccaccacaggccccgttggagccctcacagatgaggagaaacgtttgatggtggagctgcacaacctctaccgggcccaggtatccccgacggcctcagacatgctgcacatgagatgggacgaggagctggccgccttcgccaaggcctacgcacggcagtgcgtgtggggccacaacaaggagcgcgggcgccgcggcgagaatctgttcgccatcacagacgagggcatggacgtgccgctggccatggaggagtggcaccacgagcgtgagcactacaacctcagcgccgccacctgcagcccaggccagatgtgcggccactacacgcaggtggtatgggccaagacagagaggatcggctgtggttcccacttctgtgagaagctccagggtgttgaggagaccaacatcgaattactggtgtgcaactatgagcctccggggaacgtgaaggggaaacggccctaccaggaggggactccgtgctcccaatgtccctctggctaccactgcaagaactccctctgtgaacccatcggaagcccggaagatgctcaggatttgccttacctggtaactgaggccccatccttccgggcgactgaagcatcagactctaggaaaatgggtactccttcttccctagcaacggggattccggctttcttggtaacagaggtctcaggctccctggcaaccaaggctctgcctgctgtggaaacccaggccccaacttccttagcaacgaaagacccgccctccatggcaacagaggctccaccttgcgtaacaactgaggtcccttccattttggcagctcacagcctgccctccttggatgaggagccagttaccttccccaaatcgacccatgttcctatcccaaaatcagcagacaaagtgacagacaaaacaaaagtgccctctaggagcccagagaactctctggaccccaagatgtccctgacaggggcaagggaactcctaccccatgcccaggaggaggctgaggctgaggctgagttgcctccttccagtgaggtcttggcctcagtttttccagcccaggacaagccaggtgagctgcaggccacactggaccacacggggcacacctcctccaagtccctgcccaatttccccaatacctctgccaccgctaatgccacgggtgggcgtgccctggctctgcagtcgtccttgccaggtgcagagggccctgacaagcctagcgtcgtgtcagggctgaactcgggccctggtcatgtgtggggccctctcctgggactactgctcctgcctcctctggtgttggctggaatcttctgaaggggataccactcaaagggtgaagaggtcagctgtcctcctgtcatcttccccaccctgtccccagcccctaaacaagatacttcttggttaaggccctccggaagggaaaggctacggggcatgtgcctcatcacaccatccatcctggaggcacaaggcctggctggctgcgagctcaggaggccgcctgaggactgcacaccgggcccacacctctcctgcccctccctcctgagtcctgggggtgggaggatttgagggagctcactgcctacctggcctggggctgtctgcccacacagcatgtgcgctctccctgagtgcctgtgtagctggggatggggattcctaggggcagatgaaggacaagccccactggagtggggttctttgagtgggggaggcagggacgagggaaggaaagtaactcctgactctccaataaaaacctgtccaacctgtggcaaaaaaaaaaaaaaaaa

SEQ ID NO: 21 - protein, 325 aa, Homo sapiens
  MISC_FEATURE: Genbank Accession No. NP_060502 for SEQ ID NO. 21.
MSAQAQMRALLDQLMGTARDGDETRQRVKFTDDRVCKSHLLDCCPHDILAGTRMDLGECTKIHDLALRADYEIASKERDLFFELDAMDHLESFIAECDRRTELAKKRLAETQEEISAEVSAKAEKVHELNEEIGKLLAKAEQLGAEGNVDESQKILMEVEKVRAKKKEAEEEYRNSMPASSFQQQKLRVCEVCSAYLGLHDNDRRLADHFGGKLHLGFIQIREKLDQLRKTVAEKQEKRNQDRLRRREEREREERLSRRSGSRTRDRRRSRSRDRRRRRSRSTSRERRKLSRSRSRDRHRRHRSRSRSHSRGHRRASRDRSAKYK

SEQ ID NO: 22 - DNA, 2082 bp, Homo sapiens
  misc_feature: Genbank Accession No. NM_018032 for SEQ ID NO. 22.
gaaagagccgagtgggctcgaggccgacgcgaccatcgtttgtcgacgccgctgccaccgcctgcctgagagaagtcgtcgcggccgaccccgtcgcctccgccggctaccatgtccgcccaggcgcagatgcgggccctgctggaccagctcatgggcacggctcgggacggagacgaaaccagacagagggtcaagtttacagatgaccgtgtctgcaagagtcaccttctggactgctgcccccatgacatcctggctgggacgcgcatggatttaggagaatgtaccaaaatccacgacttggccctccgagcagattatgagattgcaagtaaagaaagagacctgttttttgaattagatgcaatggatcacttggagtcctttattgctgaatgtgatcggagaactgagctcgccaagaagcggctggcagaaacacaggaggaaatcagtgcggaagtttctgcaaaggcagaaaaagtacatgagttaaatgaagaaataggaaaactccttgctaaagccgaacagctaggggctgaaggtaatgtggatgaatcccagaagattcttatggaagtggaaaaagttcgtgcgaagaaaaaagaagctgaggaagaatacagaaattccatgcctgcatccagttttcagcagcaaaagctgcgtgtctgcgaggtctgttcagcctaccttggtctccatgacaatgaccgtcgcctggcagaccacttcggtggcaagttacacttggggttcattcagatccgagagaagcttgatcagttgaggaaaactgtcgctgaaaagcaggagaagagaaatcaggatcgcttgaggaggagagaggagagggaacgggaggagcgtctgagcaggaggtcgggatcaagaaccagagatcgcaggaggtcacgctcccgggatcggcgtcggaggcggtcaagatctacctcccgagagcgacggaaattgtcccggtcccggtcccgagatagacatcggcgccaccgcagccgttcccggagccacagccggggacatcgtcgggcttcccgggaccgaagtgcgaaatacaagtaactactctgactccttcggtagctgcaaccaggagtgagcccttctctgtgttcccagggtctgctgagggccgtgtctggtggggatggggctgggctcaccctcaggagtagggctggggagtcgtgaacgggactcaggtgtgggaagaggcgagagggctgtggaggagctcgcacggcgccaggtgatgggctgcacaggcactgtcccctgcctgcgtcctggggcctgtgcactgttgcgtccatgctcagagtggctgagacttgtgtcctgaccaggccctgcttacctctgttttggtttttgtttttgatatttttttttccattgtgtttttacgtagtgtcatgttctgtgcatatagtgttgtattctcctttgcactgtttatgttacagtgaaggctctccttattaaaaatcttcgcaaaggtcactttttaatggctatctaacactccatatgtggtgggcaagtctggttggcctccggggggtttccaggtataggggatgtagggccttgcctggcctggtctgcgggtccatcgtcagtgcctgagcggccagcagaaggggggcagcagcctccactgagcctctggttcccatttcccaggttctccagagagcgggcatccagagaggagtcctgggagagcgggcggagcgagcgagggcccccggactggaggcttgagagctccaacgggaagatggcttcacggaggtcagaagagaaggaggccggcgagatctgaacccgtctcccgggtgctgtaaatagtctgataaacgttcacacagtctaaaattaccctttatatttgctgaatacaactcatcttttgtagtttaaaatttctattgttttggagctagctgtgagtttctagaagtgtacagagttgctcctgtgttcccgggtcatgttgagtaggaataaataaatctgatgctgcctcctgaggctgcggggggtttctgc

SEQ ID NO: 23 - protein, 1362 aa, Homo sapiens
  MISC_FEATURE: Genbank Accession No. NP_490597 for SEQ ID NO. 23.
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 24 - DNA, 5198 bp, Homo sapiens
  misc_feature: Genbank Accession No. NM_058243 for SEQ ID NO. 24.
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaagaaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggta

SEQ ID NO: 25 - protein, 371 aa, Homo sapiens
  MISC_FEATURE: Genback Accession No. NP_958815 for SEQ ID NO. 25.
MSAQAQMRALLDQLMGTARDGDETRQRVKFTDDRVCKSHLLDCCPHDILAGTRMDLGECTKIHDLALRADYEIASKERDLFFELDAMDHLESFIAECDRRTELAKKRLAETQEEISAEVSAKAEKVHELNEEIGKLLAKAEQLGAEGNVDESQKILMEVEKVRAKKKEAEEEYRNSMPASSFQQQKLRVCEVCSAYLGLHDNDRRLADHFGGKLHLGFIQIREKLDQLRKTVAEKQEKRNQDRLRRREEREREERLSRRSGSRTRDRRRSRSRDRRRRRSRSTSRERRKLSRSRSRDRHRRHRSRSRSHSRGHRRASRDRSAKYKFSRERASREESWESGRSERGPPDWRLESSNGKMASRRSEEKEAGEI

SEQ ID NO: 26 - DNA, 1452 bp, Homo sapiens
  misc_feature: Genbank Accession No. NM_201412 for SEQ ID NO. 26.
gaaagagccgagtgggctcgaggccgacgcgaccatcgtttgtcgacgccgctgccaccgcctgcctgagagaagtcgtcgcggccgaccccgtcgcctccgccggctaccatgtccgcccaggcgcagatgcgggccctgctggaccagctcatgggcacggctcgggacggagacgaaaccagacagagggtcaagtttacagatgaccgtgtctgcaagagtcaccttctggactgctgcccccatgacatcctggctgggacgcgcatggatttaggagaatgtaccaaaatccacgacttggccctccgagcagattatgagattgcaagtaaagaaagagacctgttttttgaattagatgcaatggatcacttggagtcctttattgctgaatgtgatcggagaactgagctcgccaagaagcggctggcagaaacacaggaggaaatcagtgcggaagtttctgcaaaggcagaaaaagtacatgagttaaatgaagaaataggaaaactccttgctaaagccgaacagctaggggctgaaggtaatgtggatgaatcccagaagattcttatggaagtggaaaaagttcgtgcgaagaaaaaagaagctgaggaagaatacagaaattccatgcctgcatccagttttcagcagcaaaagctgcgtgtctgcgaggtctgttcagcctaccttggtctccatgacaatgaccgtcgcctggcagaccacttcggtggcaagttacacttggggttcattcagatccgagagaagcttgatcagttgaggaaaactgtcgctgaaaagcaggagaagagaaatcaggatcgcttgaggaggagagaggagagggaacgggaggagcgtctgagcaggaggtcgggatcaagaaccagagatcgcaggaggtcacgctcccgggatcggcgtcggaggcggtcaagatctacctcccgagagcgacggaaattgtcccggtcccggtcccgagatagacatcggcgccaccgcagccgttcccggagccacagccggggacatcgtcgggcttcccgggaccgaagtgcgaaatacaagttctccagagagcgggcatccagagaggagtcctgggagagcgggcggagcgagcgagggcccccggactggaggcttgagagctccaacgggaagatggcttcacggaggtcagaagagaaggaggccggcgagatctgaacccgtctcccgggtgctgtaaatagtctgataaacgttcacacagtctaaaattaccctttatatttgctgaatacaactcatcttttgtagtttaaaatttctattgttttggagctagctgtgagtttctagaagtgtacagagttgctcctgtgttcccgggtcatgttgagtaggaataaataaatctgatgctgcctcctgaggctgcggggggtttaa

SEQ ID NO: 27 - DNA, 23 bp, Artificial
  Synthetic
ggagatggcacaggaggaaagag

SEQ ID NO: 28 - DNA, 22 bp, Artificial
  Synthetic
tgtgagccattggtgtctttgc

SEQ ID NO: 29 - DNA, 25 bp, Artificial
  Synthetic
ggccactacactcaggtagtgtgga

SEQ ID NO: 30 - DNA, 22 bp, Artificial
  Synthetic
aggctcatagttgcacaccagc

SEQ ID NO: 31 - DNA, 20 bp, Artificial
  Synthetic
acgcagagcgacacaggaag

SEQ ID NO: 32 - DNA, 18 bp, Artificial
  Synthetic
gctcgtcctgcacccaca

SEQ ID NO: 33 - DNA, 25 bp, Artificial
  Synthetic
gaaggaaatgtggacgaatcccaga

SEQ ID NO: 34 - DNA, 25 bp, Artificial
  Synthetic
gctgaacaaacctcgcaaacacgta

SEQ ID NO: 35 - DNA, 19 bp, Artificial
  Synthetic
gctgaacctccctgattac

SEQ ID NO: 36 - DNA, 20 bp, Artificial
  Synthetic
cattcctgagcattccagta

SEQ ID NO: 37 - DNA, 21 bp, Artificial
  Synthetic
gtggtacgtgttggtgaagga

SEQ ID NO: 38 - DNA, 20 bp, Artificial
  Synthetic
gtagctgcccatgacctctt

SEQ ID NO: 39 - DNA, 18 bp, Artificial
  Synthetic
tcaccatgtctggaaacc

SEQ ID NO: 40 - DNA, 23 bp, Artificial
  Synthetic
ggagatggcacaggaggaaagag

SEQ ID NO: 41 - DNA, 22 bp, Artificial
  Synthetic
tgtgagccattggtgtctttgc

SEQ ID NO: 42 - DNA, 17 bp, Artificial
  Synthetic
tctatggtgagcgcttc

SEQ ID NO: 43 - DNA, 21 bp, Artificial
  Synthetic
ccggaggaccttatccatgtt

SEQ ID NO: 44 - DNA, 22 bp, Artificial
  Synthetic
gctcatcttgctcttccacaga

SEQ ID NO: 45 - DNA, 18 bp, Artificial
  Synthetic
tttccaatgaagtcaccc

SEQ ID NO: 46 - DNA, 18 bp, Artificial
  Synthetic
agtatgttcccgcttgtg

SEQ ID NO: 47 - DNA, 21 bp, Artificial
  Synthetic
acttgactctgtaagtcctgc

SEQ ID NO: 48 - DNA, 19 bp, Artificial
  Synthetic
ggtcctggcttccttccat

SEQ ID NO: 49 - DNA, 18 bp, Artificial
  Synthetic
ggctgacgacagcacagg

SEQ ID NO: 50 - DNA, 18 bp, Artificial
  Synthetic
aaagagcacggcggtaag

SEQ ID NO: 51 - DNA, 18 bp, Artificial
  Synthetic
tttcttgcgtctgcctgg

SEQ ID NO: 52 - DNA, 19 bp, Artificial
  Synthetic
ggaacattagccattagca

SEQ ID NO: 53 - DNA, 20 bp, Artificial
  Synthetic
tgaaatgacgagagcaatag

SEQ ID NO: 54 - DNA, 23 bp, Artificial
  Synthetic
gcttagagttacacatttgctaa

SEQ ID NO: 55 - DNA, 21 bp, Artificial
  Synthetic
agagtaacctgaatgtggaga

SEQ ID NO: 56 - DNA, 18 bp, Artificial
  Synthetic
gtaaggacgctcatcatc

SEQ ID NO: 57 - DNA, 18 bp, Artificial
  Synthetic
aaaagtgccaggtaagtg

SEQ ID NO: 58 - DNA, 20 bp, Artificial
  Synthetic
tttgttgggcagagtctatg

SEQ ID NO: 59 - DNA, 19 bp, Artificial
  Synthetic
caggcgtaggtcagtcaat

SEQ ID NO: 60 - DNA, 19 bp, Artificial
  Synthetic
tcttctcttgggacctcac

SEQ ID NO: 61 - DNA, 21 bp, Artificial
  Synthetic
gcagttctgtctacaagtcca

SEQ ID NO: 62 - DNA, 18 bp, Artificial
  Synthetic
tctgaccagttggtgctt

SEQ ID NO: 63 - DNA, 18 bp, Artificial
  Synthetic
gaatgggtgctccttaca

SEQ ID NO: 64 - DNA, 20 bp, Artificial
  Synthetic
tgaatcttgagtggacctgc

SEQ ID NO: 65 - DNA, 18 bp, Artificial
  Synthetic
tcttccagggcaatgagg

SEQ ID NO: 66 - DNA, 19 bp, Artificial
  Synthetic
gtgttctccctggtaatgg

SEQ ID NO: 67 - DNA, 20 bp, Artificial
  Synthetic
cctttcaactgtgtctccaa

SEQ ID NO: 68 - DNA, 19 bp, Artificial
  Synthetic
ctcctcaggcagttcttct

SEQ ID NO: 69 - DNA, 20 bp, Artificial
  Synthetic
gcaagagcacacatacacag

SEQ ID NO: 70 - DNA, 18 bp, Artificial
  Synthetic
tggaggagagagtgagca

SEQ ID NO: 71 - DNA, 19 bp, Artificial
  Synthetic
cttaggtgaacgcaatgag

SEQ ID NO: 72 - DNA, 18 bp, Artificial
  Synthetic
gacagtggcaggtagtgc

SEQ ID NO: 73 - DNA, 19 bp, Artificial
  Synthetic
aacctgggctatgtgagac

SEQ ID NO: 74 - DNA, 18 bp, Artificial
  Synthetic
cggcagactttagaccag

SEQ ID NO: 75 - DNA, 19 bp, Artificial
  Synthetic
gccctcagtttcttctttc

SEQ ID NO: 76 - DNA, 18 bp, Artificial
  Synthetic
gcaagcgtgtgtgactga

SEQ ID NO: 77 - DNA, 18 bp, Artificial
  Synthetic
ggtgctggatgctgtctt

SEQ ID NO: 78 - DNA, 18 bp, Artificial
  Synthetic
tgtcagtgggcattctca

SEQ ID NO: 79 - DNA, 20 bp, Artificial
  Synthetic
gagattggaacctgtcattg

SEQ ID NO: 80 - DNA, 19 bp, Artificial
  Synthetic
gcagagttcctgacagagc

SEQ ID NO: 81 - DNA, 18 bp, Artificial
  Synthetic
tgatgtggtgtttgagcc

SEQ ID NO: 82 - DNA, 20 bp, Artificial
  Synthetic
attagcctttgtgtgtgtgc

SEQ ID NO: 83 - DNA, 21 bp, Artificial
  Synthetic
tgcctaactgactaatctgga

SEQ ID NO: 84 - DNA, 21 bp, Artificial
  Synthetic
tgtatcttaggtgtctcctgc

SEQ ID NO: 85 - DNA, 19 bp, Artificial
  Synthetic
accaacagcactcagtcct

SEQ ID NO: 86 - DNA, 24 bp, Artificial
  Synthetic
attgggaaagatttggatgtgctc

SEQ ID NO: 87 - DNA, 26 bp, Artificial
  Synthetic
gtaccttatgatgatgatgagttgtt

SEQ ID NO: 88 - DNA, 24 bp, Artificial
  Synthetic
cactttacattcttccttgtttga

SEQ ID NO: 89 - DNA, 21 bp, Artificial
  Synthetic
caggtccttactttgttccga

SEQ ID NO: 90 - DNA, 19 bp, Artificial
  Synthetic
cttctggcttcccaacacg

SEQ ID NO: 91 - DNA, 19 bp, Artificial
  Synthetic
gggcatacggttgttgagc

SEQ ID NO: 92 - DNA, 21 bp, Artificial
  Synthetic
gtgaaggaccagaagaggatg

SEQ ID NO: 93 - DNA, 18 bp, Artificial
  Synthetic
caagattagcctcccgca

SEQ ID NO: 94 - DNA, 22 bp, Artificial
  Synthetic
aggaagataatcaccgaggagt

SEQ ID NO: 95 - DNA, 25 bp, Artificial
  Synthetic
cagtcccatacaaagaacaagatac

SEQ ID NO: 96 - DNA, 22 bp, Artificial
  Synthetic
tgtgctgtgctaaacttgtgaa

SEQ ID NO: 97 - DNA, 23 bp, Artificial
  Synthetic
attctgctaaagtgtccatcaaa

SEQ ID NO: 98 - DNA, 18 bp, Artificial
  Synthetic
tggacgtggcctctgcac

SEQ ID NO: 99 - DNA, 19 bp, Artificial
  Synthetic
caccacctgcagcctgaaa

SEQ ID NO: 100 - DNA, 18 bp, Artificial
  Synthetic
agggctttcagcccagag

SEQ ID NO: 101 - DNA, 16 bp, Artificial
  Synthetic
agggctttcggcccag

SEQ ID NO: 102 - DNA, 24 bp, Artificial
  Synthetic
gaaatcaccaagatgcaaatcatg

SEQ ID NO: 103 - DNA, 20 bp, Artificial
  Synthetic
ggcctcctccagagcttctc

SEQ ID NO: 104 - DNA, 17 bp, Artificial
  Synthetic
agaaagacccccaggcc

SEQ ID NO: 105 - DNA, 20 bp, Artificial
  Synthetic
ttaagaaagatccccaggcc

SEQ ID NO: 106 - DNA, 19 bp, Artificial
  Synthetic
gctgaacctccctgattac

SEQ ID NO: 107 - DNA, 20 bp, Artificial
  Synthetic
cattcctgagcattccagta

SEQ ID NO: 108 - DNA, 5198 bp, Homo sapiens; identical to SEQ ID NO: 24
  misc_feature: homo sapiens BRD4 (long transcript)

SEQ ID NO: 109 - protein, 1362 aa, Homo sapiens; identical to SEQ ID NO: 23
  misc_feature: BRD4 isoform long [Homo sapiens]

SEQ ID NO: 110 - DNA, 3149 bp, Homo sapiens; identical to SEQ ID NO: 18
  misc_feature: Homo sapiens BRD4, transcript variant short, mRNA

SEQ ID NO: 111 - protein, 722 aa, Homo sapiens; identical to SEQ ID NO: 17
  misc_feature: BRD4 isoform short [Homo sapiens]

SEQ ID NO: 112 - DNA, 34723 bp, Homo sapiens
  misc_feature: BRD4 gene - noncoding strand (homo sapiens)
tcagaaaagattttcttcaaatattgacaatagatcactctggaaattcatgtcaatggtagctgccatctggaggagaaaggaaggagggagtcaggaggatgacctagccaccctgcagctacaagccctcatacccgctaccagcagtcagccccgtagccctccccgtggctgacccctcatagcgctcaccccgtccacacagcactcagggccctacagctgagtccagaggaccacatgccgaccagcagggacggggctcccccgctgcccctccctgtccaggctccagtccccctttcccagctccctcaggagctaatccttagaccagggtccccaccaggcctccagactcacggcttcccggcgtcttcgctcctgctcccgcttccgggccaactccctctgctggtccagcatggactggggctgggagctctgggcctgtggggtggcggcggcagccaccgcagctgcttgctgttgctgctgctgctgttgctcctggcgctgctgctgctgctgctcctggcgccgacgtgcctcctcatgggcccgccgggcctgctccagcgcatcctcgtcctctcggctcctgggcagagggtcccagtcagcctggggactggtgtggccccaagagtccccatgccccacccagacacccgcccacctcatgcgctcctgccgcagccgctccttctccttctcagcgtgctcggcctgagccttcagggccttctcacgctcctctttctcccgagcggcgcggcggaactgctcgaagctgtcgctggatgacttggctgtggaggagggggtggtcggatgcttctgcactaggctggcccaggagcccatgttcttgattttcaggtcctgcagaacagagaggttggggtgggtgaggggtctgctgtgcctaaagggcatagctgggggtgtgcccagcatggcaccttccagggccaaggggcaagcgacacccatggacctccatcccaacctttttgggcgcaactggagtcttcggctcctgtttctgtttgtccttgtcaggggcccctggtggcggtgcgttctgctctgggggccggatcacaggcctcccgacatccacaggcttcatttccggccctggaacataaacagccggtgggccctggcccacctcaccccagtggggcatggtccaccagccccacagcaagcttatgtccaacacgggcctcggggggcctgagccctggctgtgggcagggagagcactcacgctggggcaggtggacgggggccttgatgctctccgggtgcttggggggctccggccgcagcgaggggctgaagggctcgctgcggatgatgggtgagtggatcttctcctccttcaccaccacgaggggctggggctggaccacggaggcagcacgcagctcctgggatggcacaggcacagcggccggtgaggtgggcaggcacccccggccctagcccacaggactatggcccagccctgccagttacctgtttcttaggctggacgttttgctggggtggagactggtgggtcaggctctggaactgtgacatctggggggaatgtatcataagcggggagggggcttcgcggaggtgacctaggagaagggacaaggaatgtgtcaaggggctggcttagaactgctggccctgagagaattgtcgggaaggaaaacgtgggctgtatggagaaactgcatgtgccgcccaggccctgggacaagggtccattagcccagctcaaaaagggtgaagacccacacaagtgactgggcctggtgaggacatgacccctccccgaaagggctgagtgccctgtgtccccctgcatgtggggcataactcagtgtgcagcagagctttctagggccctgtggcaccagtcctggtgtttgaccctcctgtgtgcatcagaatcatctgggatactgtggaaaacacagatcctgacacatgtgggcttggcagggcctgggccctaatactttttaaacccacaaagctcagcctgggttcctgccaactagcctggcccacctggatgattgcagggggtgcaagacccagtcctgggggtggggatgctcaggaccctccccacagtcctattccccactcagcctgcacgcccccacggaagagagaagcctggaattggcctccacctgcgccgagtggggagcaggcggaaaagggcctggagctgtctcttaggaaacaggccagcctgacacctgcatccaacccaaggctgggaaggatccagcagccacagggactactgggccctcaatcacagtgagccagcacctcgggactgtgcagctggagcagctgagtcctacccatccaacatgtggcctctctctgctggaatccctgcaggcaaggggcctcaccgctctttggctagctgcagctcgggacaagccacactgtccacagaggaggctgcccctctgcaacgcttttgctcccagcctagcctcatgcccatctccttccactgtctgtcagcacaggcacccttgggttctccaggagccctgctggctggaggcatctattttcataggcctggagcaaaccacaatgccccaggtgtggtagatcctctctagagccaggacccactcctaacgccagaagacactgcagctgcttggagcccacatggtggccaggcccatcctgtccccgcagcccactgctgtgtgtggctggttttcctggcgttgacatctcctccaattctagcacatcgggaccactttgggcactgccaattccattcagtgctggagggccccaggtctggcctgccagggatccacaggcacatgactgcaacagcctcccacccaactagggcagtgccttcagcacctgctgaggctgacctgggcaacggtaggctccaggggctgtggtttatagaaaccacctgcctgctggtgggttcagaggatggctcacggatgactcctgaaagctggctgggggcccagggatgcctgttaagtctctttgggagtctaacaacttggcttttggcctacaaagcccctagcaggatcccaatccacactgctcttgatgccccatcctagcctgctggggcttgggtcacggccagggacctgaagtagagctggctggagaagacctgcctcagccccgataggtgcctggtgcacccgagtgctggctcttctaaatgccttgcatcccctgtctatcacccccaacaggagtccagccctgcttcgcatgagaacaggcacagccgcacagctgccaggcccaacaccagtatttcccagggaggacaggcagcttgttcttggctcagggcatgcagagatccacttggcacctgactttttttttttttttttttttttttgagatagagtctccatctgttgcccagactggagtgcagtggcacgatctcagctcactgcaacctccgcctcccagactcaagcgattctcccgcctcagcctcccaagtggctgggattacaggtgcctgccaccatgcctggataatttttgtatttttagtacagatagggtttcaccatcttggtcaggctggtcttgaaactcctgacatcaggtgatccacctgccttggcctcccaaagtgctgggattacaggcgtgagccaccgcgcccggcctgcatccagcagcttttgctgggttaggagtaatcacagccctgcctcgttgcttgatttgcaggcagcacccggtacacacccaagacaggcagcaccatctacactgctgaggccaggaccctccaccctaaccctgcagcaccacatcaggaggcactgaccagaacatcccctgccggagccagccaggacattctggttactgacacttaagcgcccaggaaaagtgaaagggtgtggcctgaccaatgctgtcaatgggaaaagggtggcgtggactgggctggaggctggtaacaggcgatgaggacaggtcctgggcgggctctgagctgccttttgagcttccagctcagtgctccccacaccatggttctggctctccctgaacttcattcttcactctgctaagttagctgctacctgtctgcgatcactgcctcttggaagcatatcctgaccccctgaggtgcatcaggtacctcccctgggctcctagcttccccacctagcaagaagtcacctagcattgtcgtgctcctgactcaagagagggaccagaggggccttattctgagtgatctcacctcacaagcaagagagacacctgataaacacacactgtaggaagcaccaatgacccttccaggtccccctccaagctgaggctcagctctgaacccactgcagcctgaagcatgagttaacccttctggcttcctgaggacaaaactataggcccagcaccagcctccccagagtctacgggtgaggaccacttaccggttgagtaggggtccgacttgtggtgccggggtgaatggtggtgctggatgacttgctgaggcttggcaggctgcggcgggggtggctgctggcctgggggcggatgggggggctgctggccctggggtggcgggggctgttggatgtgggtggaaaactgcatgggctgcaagtgcacgggccgtggagggggctgatgctgctgctggggtggaggctggggctggggtggtgggggtggtggcggctgctgctgcagctgctgctgcacagaggggtggggtgggggcggcagggggggtgggggctgggactgcaccttcacggaagggagtagcggcgtagggggctgcaccttctgcagctgctgcaggtacagctgcatctgcatggaggtgaggggtggggcaggtggctcttcatcctccagcagcacttgggggggttgggccatggggggctgtgggagcaggggtgtttgggtcaaggctggtgacacggctgggggccgggcgggcttgggaggcagggcagcggctcggttgctgggccgtgatggctgctggggtagtgcgttgtgcaaagctggaagaacacaacaccgaggcggtgaggcctgagcacctgtggccccagcctggcctttctgctccatcctgacctcatctggactccagtgctctcctactggcctccctcaaactggcactccctccccacctaccgtggcctcccacaaactccagagcagtaaacagcttccagagagagctttagggcggtgtcatcctgccctatgctctgtgtccccagctcctgctccccatgctcacaatggcccttcacgcctcagtgcagaactcggtctctcacttgcccggccattccccaggcccccaggaaggtggccctgtccccatatcccccaggtgagtgctgtccaggttccatgggaggaatcccatctcatgagtgcatcccccaacccctgatgagaaaagctgcagaggggtcccagcttttcccatctaacatgacgcctggcccagcagcagaaataagtcaaaatcatctagattgttactcaaatgatgtcagtgttttagaaacaaaaatcttctgaatcaacgtgttaataactccatgcatgacctgtcttgaggcatctcctttaacttccaagatggcctcgagaagccacagatcttccctctagagaccagagcagctgaggctaagaagctgcccagccagagtgctcggacaccacatgggtaaaccgaggcaggaaggggtctcaacccacactggggcaggccacggctcacctggaggagagaccactgcgtgctggttgagatggggtggagtgctgtgctcaggcggctggggcaggtgagggggcagctcaggctgcggcaggtgcaggatgggctgggtgaagtggccgatggggtcaaagacgctgcctgggagctggggctccaggacgggcacctgggtggcaatgaagggtgggggcgaggacttcatcgccggggctgcctgctgcggcatggagggtgggggaggcgggggtggcggctgctgttgctgctgcggaggtggaggcggtgggggctgctggggaggcgggggcggctgctggggcacaggagccggggcctgctgcatctgctgatggtggtgatgatggtgctgcagacagagagacagacagacagacaggctgatgtcaggcaggcagaactggcccgggccagacccaacgtcccacctaatgaaggatgcccctgagcccatgtaccttcttctgctccctcccggggtgccccttcttttttgacttcggagccatctctgcagaggaaaagagaaggtagtgaggctctgggggagaaggtgagtgagctggccttggatgaagaaagacgagaaaacagggcactttcaggagaaggaccagtgaccctgagaaagggcagccgagttcaatgagggatgagttggtcatcacgggagagggagagacatgcgaggcatcacctacccacttgcctgctgcttttcctagcagggactgtgcctgaaaaagcctcccctgccaagcttcagatcaccacggttgcaccggaagctgagagcttgcccaacgcctcccactactcaagggtcagtcactcactatagtgactgagtgcatggcctggacagcaacctgctcagttttccaacaacctgtgatggttgcaatggtcacattcatcttacagctgagaaaagtggggcttcagaagggaggggacttgtccagggtcacatggctaaaaagcagcttctcgggggcctccaactgcatggccctctaaggcccactcctttccatcctccaaagggaagcagagaggctcagctggggctgagagccacaggacggtgaggaacatatcggcaagggaaaacacagagagcccactccgggagaaaaaaaaggagcaggctggcctcctgggaatgaccagctactaaagaggtagatggacctcagaagtccctgctttgggacacagtcaccatgctgcagacagaaggcagcatacgcccccactcgtgtgggccgacggcttggcaccacagtagggtggatgggtccagggtgtgtctgcccctgagaggtgtactcgagagccagcgtgcccacctctgtcctcaccccagagatttacactaagggacttttgccagttggatgtcccctcaccaaacatggatggtgtacaagccccacagggggacatgagccctggccagtgctcatgggctggcaggtcacagtcgcctgccaccagatgggaggccgggcccatggtatggcgccttttggattcctgtcactgagaagcacctcatggcactgcctggcttcgcctgagctccttgtcagggtgctgcctcttgctgctggtgttggaggacacatgctttgcccaaatccaggctccttccacttggatctgcttcgtggagggccacgctaagcttctgtgaggcacgtggccctcagtgcagcatgccaccaaaagggatggggaaggaccttgtgccctgtaagagctcaccactccacgccagacaagacacagacacagacctcagcagctacagacacacgtcctcgtacaaatgaccaggggctgcaactggggacacgggggaacctggggccccgcctaaaacctgcatacagggctcaggacgctctgggtcagagtatgacatctgatgagagagatactgttgtgggtgagacatgccagaagggaaagcttgagtagatttgcagtgtcagggcaacagggagtctgactgctgtggtggtggggggaggagcgctggctgcagaggttggcagccacaggaccagggggaccctgcacactagggaactgggaagaaaaggccagggcttctctcctggcacaaggaggtgatggagccgcgctgtctgggggcatgctgggcacaccttggaggggacccggggcagaaggcggaaggtagggggaagcagcagggccaccgtgtgaagtccgactaggagaagcctcagggatgtgtccagatatagagttgagggaaaaagtgtgtgtgcaagggaaggatatgctggggctcgcctgttgtctcgtggccttggaagaaaccttgtttttttggttgtaaaagagggatggtcactctaacccctcacagcctgcaggctgtgagcagaggcccataggagaagatgtagggacaaagcgcagcagggcctgtggggggttccagggggcatgtctcagctgccttggaaaggccacagggaatatcctgaggacttgtctccagggccccttgcaagtgctggggcccccaggcaaggcaggcctaaaaagtgggggagagaaggcggcatgtgcacggcagacccccaggaccagatgcctaaaggggctggtggtagaggtacccccttctggcccatgtggtctgagccttgtgtcagagttaccagtaaaagacaagacaggaagtgcacagcttcacaacacaccagccccaagacacccccacacaaaactctctagaaagggttacatgaagatggcctcaagtacggtgtgatctttattccatgacaatcttcaactcaacacaaatttcatctttacataggatggtaattgtacaaagaaataaaatgttttttaaaaatccgattaaaacactgattccacccaccccccagccccactccagtgaaggaggtaatggagggtaaggtcagggcagaggagaggagtccccatctgcactgagcggccgcctcccgcccctcctcaggacccacagccagctggtgcgtcgaggcccggctggcacttcctgcacaggaggctgccaacaagaagtgagtgggcactgtcctctctggctgttcctggggcaccaagcacaggtgacagcttaagtctaatgagctttcaagttcaacaagccctgaacgttctctgccagatggaagccccacgggcaagaaggacagggtccctggacagcgaggttgaaaggaccctggaggagagctggcttgtggctatggcccatgtgcacacgtgtgtgcgcgtgcgtgcgtgtgtcgggggcagcatgcaggcccgacactggtaatcaggctgccatttggtccaagaaagcagaatcttcccagaaagacaatgccagcagctccccaattgtcctgtgggaatctgggcatgaaggctctcctgcctgctcctgtctcaggcaggcgggactgcaacaggaacacatcaggcgcaggccaaagggcagctgagaatagtttgtttggcaaacagagcttaactggaagctggatgcccagaaaggatcaagagcagcccaggaactcctgagcatcaatgcagggccccgggaggccctggtgaggccagggctagcccctgcttccacagcaaatctggggcagttgcagctgctcaagcccaaggagctgaggagagctgccagagttcccaggcaggtggggagcctgcacccaagggcttgcccatggtgatagcccacttccagttccagaagaaggcaggcctctaggaagcacccacaatctacactttaaaaaaggagaaaaaatgaaaagcccctgaaacgcagatgcagttcctcaggggacttttcatgtcaggcagttttctgctgagatgctggatttgtgaccaaaaaagggctggtatttatttttagagcaaaaaagcctatttgtgtctctgggagggcttggatgatgtggccacactcgcctgccccaaccccaggggctgtcgtcattctaagggactgaagacggggaaaatgttcacactgttaacaccagtcagaggtgtccatggggcagtccctgagggtctgtgacaccctctctggtccaccagcccagactcaccccactagaggaggcgggtggggtgaggtggggttggggggatgataggcccaaggcccttcaccagtgcctgcctggcaggtcctgcatactgcagcctggttccaggaggtactttccctatgtgacttgagaagacggaggccaaagaaactggggaaatgcccaccaggcaacttaagcttttggaaatgcacgggaacaatggagacgaaggaggaaagactccaaaaggctcagagtcagcggtggctcgagccttacagggcaccagtgagaagcatgctgtgggaagagcctcactgagccttccagggtctagaggccttgcctctaaagctcccactccttatctaggacagctctagaggtcagagtgttcttccgcacactgaacgagagctctcctggactgatgtcactgcaaggttcaggagggaagtagttagggtccaaagtcccagaaaaagagggatgggacaaagagagaaaacatacttggggtctggctttgcacacagagtagcaccacatgaatgcatgtgttggggattgtcatcaccctccaggagatgccacccacccagaggaccccagagaaccaggaaggctgggcaggacagccaggcagtctttacacagagaggcctgggcggctggccaggcaggcagccttcccgaaggcgggactaggcgtgtgctgctggacatgactaatccaccccgggggacaggcccagggctctgaggaattccataacttgatggagtcctgtccctttcacggaagaaaatggacttaagctatagcttgctgggaaggaatctggaactgaagaccgttttattaagagtccgtgtccaatgattaggcaggacctacgtagagggagagagaaaccagtgtgagagaaatggtgtggaatgtaccatttaagtcacaagaacagaagaaccgcagactgagccaacctcctgcgccctggtggctgatgggcacaagaacggcactggagactggagccctgccccgtgcctggctaaccctgggtagggacagacccaccgcccgcttagaaaggtcacagaggcagaatgcatggatgccttggaaaccgcccccagcctgctcagcttctagagagtggccctggttcccactagcaagctccacgtgtgccctgcctgagcgcacgccggcatttcctttactgccctcaagtggcttcttttgaatttgagtgatgcccctgccctgtcccaccagccaggtgtggcaagcccaccaggcccctgctgactctgagctcatctcacccaggggctgtgcactgtctagtctcaggaaggcgatgccagcccttcctcctgggtctacaagaaaggaggtaccagaatgcaggacccccagtggaggggaaagagcaaacagcctcatctctgcattcctccagatcccccaaacaaaaagctcaaactctaaaaagcagaacctattatatacactgttgaggaggagaaaaaaaagaaaaagaaacaggctaaggaataaagtcttgtcctttctttccttttttggggtatccaacatggctccctggaccagggaggggcactgggcttgttcccacccacgtagcagagcggatgcctctgctggagtcacggatgcaaccgacgtgctactccctggcccctgagtgcgggcatgggaaaaacccgcccactggcggcctctcactctgggccacattccacaaggctcgccctcacccgcccatgaagccccagaatccctccagctgcaaatgctggctgtttgtggggggagtcacctggctgtgcagacagaaaggctactgccctatcttcaagtctcttcctactcccccacctccagggtcctcaccattcccctaggtaagcgccacactctggagcaggcaagcagcaggcctggagatggactaagtccctcaaagaccctgggggtcaggaagctgaggctggttgtgtggtaagaacgccccatggcctctgtctgcacttcccagctcaaatctttccaaaacatcaagcattcctttttcccctggagctattaagaaacacccacatgctccaatcctgggaaaaggaagccaggaaactaaaatagctggactgcgctcccgttccaactagacgaccagaccaaagcccaggaggatctctcacacctccctgagaccctgtcccagcccaggtagctgaggttaaacgggtattcctaagagggacataagctgtggggacacttctgctcacaaagacctgtgcatgacacttgggaagaaccagatgtcagctatgtgacaagtgaaagctagaagattcctaggacatgccctgccccatgtgcttggcagacctaaccccagtgctaatgacagcaaagccacagtactgtccacagcccacctgggacagtgtcttcctccagcctgctccacccaggtctgcagatgccctaggggtggtgggagagtggcatttgtgccctacccctgcctgtctcaatacagaatcctcaagcagctccaggctttagctggaatcacagctcaatggaagaccacagcccaagcaatagctggaatcagaggaagtggtagggctggcccactgcactgcatgctgctggggcagagggtggggaatacatggtggcctgggacacagctgggaaagcagcacccacccccgccatcctcctcctgcctgtgttccgttgctccctgaactagagcctggccccagaggaccaccccacgcctgcatcagtccacagcatcccggtctatggatagctaccaagaggggtgttcatgtatcacaccccccagcctcaatctagtgactccattcgttttcttctggtgattcatgtctcagggccgttgtcagcggaactaaccccgtagtataagtgacaaaaataggccctttcaggaatttcggctccaatccaaatggaaacacaagaacgggggggggggggggggcgggggcgcgggcctgcaaatcaactaggcaccaaaagaccaaagggaaataatgttcccccaccctgacccactcccgttaaggcagagctgcctgtaacgggccatggacatcagggctgccgggattcctggacccaggagagcagctgctgaaggagctacgccccctctgccccccgccctgagagtgcagagtcctgtgcgggacagggcagcagaaggctgtatttactggtgccagggtgtgggttcccatttcgggatctgccgtacgaaaccctcgcagcggcccctcctgagaggagaagcaccctgccggcccacgacagagctcactcagtgcacacagaaggcaaaggaaagcccctgctggccaggattgggaagacaaaagggagccaggcaaaacccaaggtggggagggcgcctgggaagaggggaaggccagagcccagatagctgcatgagcggccagggctggaccgccagagcctgggagcctgggagccagcggctctgagactgtaaatctcgccctccttcccgccaggacagctgttcccaggactctcagattacttgttgcacaatctttccacttttttttaaagtgggggagtgaaaaaataataaaatctcggcagcggcaggaggggtgtggggcactcgttcaggaaaccaggagcattcccactcctctcccaaagcagaggagcccagaagagggacgcagaggccaagggcaaaggccatgatcacacactggaaatcaggatgtgatggccacagccctctaaggtgtcttgtcttgtgagggctaaagcagcacactgccacgaccacgtcatgtcattttagttaatcaggtcccctgacaatttcagcatctggggtttccacttggtcactcagatcctcagatgagaagactacaaaacccctggtaggagaagtcaactagaaaacaggaggtgcaaaaacccagaggaaggcaagctggaggcttggctggcatggaaagtgaaaagtcagcggccaccaggaatgctaattggatccaagaatacagccattcaccccagtcactctgctgcgagtgggggccctagcttcgggtggcaaagccagcccttcctgggcagccatggggtgcagtgcctcctgctggctgatggattacaaaagcagtgtggggagagtaacggttagagatgaaagggtgaaggatggcgacagggaaggccaggaggccagtgagctgcccacggcattctgcatgcagttgtggtagagaatgtgggcatctggagtcctggagggtccttcagcccacttcaaggcaagtctcctcactcacctgggaagcagagggcaccaggctgtccaagggaagaggaatactgacaaggcccgaggtgccacctacccccaccccatctgtgcacacacacctcagagcaacatggctctccccagctgccgtgaagggtcctcaatcttccttggcacgatcaagttatttacatgcactgattattgagggttagcatttatttctagttagaataaaatcccagggatgggacaggcccgtgggagaacatgcaggagtctactgccaaaacacaggtttcccactgtggatccccactccccagtctgcctgagaccctagggccagctctcaggtggtctgcagcacgccagctcactgacagctgcaccagcctagcggcaaccccgttggccgtaaacacaggatgttagctccatctctcagcaggggctggtcgccaggccttctgggcaccatggctccctccctagcctctcccctgcgccagcctgagcatctgcgcccctgagaataatgaggaaagtgcacacttggaggagagcagtcccctctccactcccacctggcacccagcatcactacctgcctccacctcccactgaggctgctgctcagaaggtgggctctaatggggaacccagggtccaacgtgcagacccacggggcttgaaaagaagggactgttagttagaactgcaggagggacacgcaagtccaggtgcgtgtggagttagggcagattcaggagcagccaacacagcaacgagcacactctggggaggggtaggcaatggctggggcccaggtgatggcagggccagcagcagactggcgatgcggctggccagctgcagtctgctccacatccaccagaaaccagcgaagcatctccctgaagcggccgcactgcacgtgactgtgatacggggaaggccctggggacacgaagtctccactggtgcagaaagctgggtgtggtcacatcaaggtcaacagcacagcctggacccccacgcccacagtcctcatggcccagcttccccgcccaccatccagggctccgcagtgtcaacgcccttggccattcatcctccatgaccagaccctggcagggagagcataacggccaggccgagagaatggacactactgcacagagtgcagggctattcatggccccagggagacagttaacaaagagacagacagacagaggaacccagcaagttctgggagtgccgtctctgggctctagcatcctggacacaggaccgagctagtgccaggcacaggtgggaagagtttggtaagacacgtagaacacaagtcacctaacctgtttcggagtcttcgctgtcagaggagctggactcactggagctctccgactctgaggacgagaagcccttcatcttggaggagccggcaatcacatcaactttctcagctgcaatccaagcaatgggagctggtcaggcagggctgtggcccaggaagccgctctaagccatgggcacaccccatccatctggaggaaggaccagtgaggagcctgtgcacggtgggcccaggggctgctcccaactgccagctgccactcccagaacccacgtggtcccacagttcccatgggtagactgcttacaatggaatttcaaggttccttcaatctctcaaaaagtctcctttggggggtttatcaaggattcaagagaccaggcagaaggataatttcctaacacaaggtaccgctgggcataaggcttggattctcccagttactcttctagcaggggaagaagaagatccaaaaggaacatgctgccaaaaggtaacgcaaggcagggagagacccccgggggtctctcagcacagctgttcagacgcacgctggaatcatcatgtggtgtgccccaggtaggggcccaaggaccccaggagctagtcagggctggggacaaagactgtgtgcaatgccctgacaccgatcctcagaggaaagccgtaggccaggatgagctgtgtgacacaaaagagctgcctggaagagtccagaaaggatgccacaccgaactgtccctgctagagagggacagaggctggctcagtgctgcataacgtatcggtacacaggtcctgggaccctgcagacccgcctttcctccttccccaaagaggccggaggaagactcagcagcaggtagcctgggttgaaagcagctccggcttagaacacagctgcaggcaacaaggtgcagagaggccccagacaccaccagtcagatatgtagaaaggcactgacagcctaggggtgagtgcaatgtcacaacctttcggaggtttctgtgtcaagaacacagatattataattggaaaaaaaaaagggggggggcgcagaaagagtggactgagcaaggagggaaaagttactctgagggtgcccacagaaggaaccccatgcccagggggcccaagcacaccttgaggtttccttttcttccgcaaacaggaggtgacatagcgctccagctcacgcagtgtggacggcttcagggtctcaaagtcgatttcaatctcgtcggggttggaattcttcagggagggctcccgtgactggatgatgtgcaccacgcggcccagcttctcgccggggagcttgttgatgtccaagctgagctgccgcttctcctcataggacataggcttgcacttgtcctcttcctccgactcatacgtgggagggggcttgctcttcatgggcgctggctccttcttgctacgaagggacgatgcagacaccatcaagaacgggccccctgaggaagccaggcactcattcctccacatgtatgttggggggaaggggtgcccttcccataccccccacccaccagccttcacaggaaacggggcatgggcaactcaatctctgggagcacccagtggagaccgaagcaaactgtgactggaggctgcccaagtgggggatgcactacagccccatccttgccgcctatcctccccaaccaaacatggtgagctccgaaggctcatcctacagaaagaggcagagacagtgcagctaagtcctgtgtgcaagtggccaccagcctggcctgtgaagccacggctgcagaggggcagcctccgcacccaatgaggacagggaggggcagtggcccacacagtctcagagcaagccctggaaggagggtccccacccaggacaaattcaggggacacagacctcacattgctgttgctgctattatttttcttcgtctttttaggaggaggttccttggctttgctttttttattctcttccacttcctctttccttttgtgcttttcttttttcttttccttcttgtctttctcctttttctttggtttgttctgctggggctgagagagggctgcaagctgctcgtgcacggctttgagctgtagaccagacaggcaagacacacactcagggctgaaaccaccttcctgtcacccaagacccaggcgtctgctccaaaaaacatgcgacagcatgcacctggggcatcagggtgtgggcataggggaggcaggggaagggaaggcccccagctttgcgcctgtcatgttaaccagattcctaacaagactatctccacccattcactcaactccaaagactgccagtgtcttcctcaccagaggagctgatcaagccgacagacaccaagtctcacggccatcctgatcgcagaaggagggcccctaagaatctaagtaagagcacacggacacgtcaggcacgaagggcctgatgactggctatagggagaggagcaaacaggtagccccaggagagacgaacatcctggctcaacctcctccttccagatactgaaaggatcaagccacctcctctctgcccagaaaatggccatgtccctccccatccccaaccgcaagctcaacacatatatcatacatacgtgtatgatcaacatgcatttgacctagcaaaggggaaaaggccacaggtggtccagtgggaattatgctcccttgggaactcagaaccaagaacatctcttagaaacttctggggaggccaccctggtccacggactctggggattaagaatggagccaccaatgccctcacctgctcctggagctcagccagccgctgggctcgctcctcctcagagtcatcagtcgaactgtcactgtccgaggagctatcgctgctgctgtcgctggatgagggcggggccacaaccttggtgggagggggcactgccggggaggacacggccaccactggctcctcaggctcgtccggcatcttggcaaagcgcatttcgaacacatcctggtgagggaaagacatgctgtgacggctgctgggtacccaggccgcggcctagcatcacctgccctcattggctgctggggccaactcaacagaaagcaaccgagggcgaaagaggatggtgatcctgtctctttgctgccgagacaggggcaagggccagccaagggcctctgacaactcttgcaacactcttctctcaaatctcactttgaagaacaaggtggtccagagatagcggggcactcactccctggaggacaccgcccaggcaaggacatgagacccaggccactaagacagccagctcactgaagtctgagatggtactggaccaagggcatcctcaaaccccaaaacagttagccgaggctcccaccagatgcgtgcgggggagcaggagttgtctgtggtttgagagggcattccaggcagaggacacagcacacgcagaggtccccggtgggaatgtacctgccacaagcgcacctcctctaggggactaggctctgctgtagcagacacggttctaagtcggtctcctcattcctcagatagcccctgcagcacttaaccaagcagggctgcaccactggaggaggcatctccagcacacacaggacaaggtaagagaggtttaggcagcaggtgtggggcactgaggacaggctacaggagggagccactgtgcgcaggagaccctaagacctttcaaccctaagatccagcaatcagatggaaccacgtggggtcatggagctcagggaacagcagccatgcatggacactggggtctggccacacatactgccagccagctgtcaagtccctgccctaccccgggggcgctgtgctcaatccagtgatggctcaagacagaggcagaagcacaggcctggcttcccagcaggattctgggacccgcttcaaaccaggaataaactgatcatcgtggaaacacaccatggcaaagaccttcccccaaatgtgtgtcagtgcttcagagaggaccacggccctggaatcccctcctgatggctcaggaaacactccttgaggcttcctctcccacgctgctggagcaggcatcccatataactgtggtgttgattccctcccctccaggacacccggagtatgacttcttttagccaataactttttttttggagacagtcttcacccaggctggagtgcagtggcgcaatctcagctcactgtaacctctgtctcccaagtagctggaattacaggtgcacaccaccatgcccaactaatttttgtatttttagcagaggcagcgttctaccatgttggccaggctggtcttaaactcctgacctcaacagatccacttgccacagcctcccaaagggttgggatgaccagcgtgagccactgcgcctggcccaataacttttttctttctttcttcttcttcttctttttggagatagggccttgctctgttgcccagggtggaatgcaatcaatggctcaatcatggctcactacagcctcatcctcctaggctcaagagatcctcctgcctcagcctccttgagaactgagaccaccatgcccagctaaatttttgtgcagacagggttttgacatgttgcccaggctggtctccaactcctgggatcaagggatcctcccacaaggtggtctacaatgaaaagtgccaggattacaggcatgagccactgcacccagcaaatacctttattcagaaataactccttgagcaggtctgagacaaggaagaagatacacaacaagggacgaccagaggccatcaccagttagcctcccaggagtggggcccagggaggaaaaggtggggggcttcacatcagagtggaggccccagtgagagagtaagacagatccctagccctgcagcacccggctacgtggcaggtgggtagtgccctggatgtgggtgggctcaggtcccaaaatgactccaagaggcagctggggccacatgagcttctggaagagccaggcctgctgccaagcagcaggaagcaggttagagaagtgggcctcagaagtgccaggacatcatgaagggagataatcagctatgcctcccaggacaaagggagtgccctctactgagtggtagctacctcagcgaactggtctgtaagcctgggccagcttcaagacttgaggatgtccttgttcctccccaccaggtgatgactgttgggtaaatgctggggtgggggtgtgtggcattcactctgaggcctccttccattccctgggtgctcatggccctggctctccaaaggccccaacaacaaagagcaaaaaatacaagacacaaagtcagctaagatgagaaactaacattggtgggactgggaactgccagcttcctggcgtgtccagttgtgtgaccctgtcatccttccctgtctcttggatgtgagaactcttgttgtagcctgagtggggggcatcctgggaaacccacaagccagtgggcccagggctgctgtaggaaaaccttgctgtgtggcttcaacagtcacaagtctccaagtaccagcttgatggtagacaggaatgacgagctccctgttcaaggagctcagtctagctggaaacagaaaggtgaatgaacacatgggcagcaaggggttgccatgaactggaagtgtgctgagacctaagagtgaaaagaacacagtcaggggtgctatggggccagcaagaggacagtaaagcccaattcaagatgcaccgaggcaagcctctcacaagggaggatggtgatggtgaggggtagcaggcaccagcacccgccagggaacaccgagtgcaggcattctgttgggctgacagtctcttgagacccagctgaggcccagccccctgcccggcatcccacagacctacacctcactcctctggaggtctgggtctgaggtcatgcttctcagtttcagcacacctcactggtttcagacaagtatctcagctgtcaggctgtggaacacaggttttctcaatgacccgagaatttagaggctcagagagaaaaataaagtggctccttttccaagtaactttccagataatcccacttcttgggcccagccaaattaagagacccaattaagcaggtcccaatagctcctgaagaagtgagcaagggctggaagctttgtccctccccttccctgatggagaatttgctctctacctgctgatgcagagaaagtagagagaagtaagaaaaacaaagcctgaaatgccacaaacacaactgcccactgaagagaagcctgctctggaaccctccaccatcaactctgttgctcaaacagaaacacctctaccaagcactggcagagaggtgtccctcccaagggcaggcgaaacaggctgggccagaggcagcccacctacagcaccccccaccccttgcctgcactaggcccccttgagattgaggcctgtgtccactccagtccggcacctggagcccgtgtgtaggaagaaactctactgctgcagtgtctgtggtgtgacagacatccagtggccttggtaaactctacattcaggcaggcttactcccctctcagaaggagaaaaatgcaaaaaaaaaaaaaaaaaccacttaaaacccaaaaatctttgttggagaaaatctgcgaaaaggaggtggctccagggtgtcaggagaggacagaaaaaatgagacaaaaatgaggaaggaagggctagaacacatcagagggtgctttttcttttctttctcttagtaaagtagctcaaaacagtcacctttggtgctggaagagaatttgtttctgctgagttcttcctatgagagtagatgaagggcatgggtatcaatgtgagcagtgtgggcaaaagctgcgcccctgtcttctcaagagagtgggagagagggagcggtggaggagttgaaggcaccaatctgagggcgccactgccaccgggtggccctgaacaaaaactgcattgaaattaaaaagtctggccgggcacggtggctcacgcctgtaattccagcactttgggaggcccaggcaggcagatcatgaggtcagaagttcaagaccagcctgaccaatcggcaaaaccccctctctactaaaaaacaaaaattagctgggcgtggtggcgtgcgcctgtaatcccagctactcaggaggctgaggcaggagaactgcttgaaccaggaggcggaggttgcagtgagctgaggtcgtgccactgcactccagcctgggcgacagaaagagactccatctcaaaaaaaaaagaaagaaagaaagaaagaaattaaaaagcctgaactccttcaaggatgggaaatgcaaacatgaatgatgaaggggcaatgttgtttgtgaagaaggcacacagacaacaccaaagtgaagagagggcaacaattccccaagtcacagaaaataaaactaagaaacaggccaggtgccgtggctcatgcctgtaatcctggtagtatgggaggctgaagcaggtggatcacctaagcccaggagtttgagaccagcctgggcaatacagtgacatgccatctctaccaaaatacaaaaattagccaggcatggtggcacacgcttgctgtcctacctgagaggctcaattgagcacagcaggttgaggctacagtaagcagaatgacaccccgtttccaaaaataaataaataaaaaaaagtagaaataaaaagctccaagcctgagcatgcatttaaataacaagagccacatccacactagtaagaaagagaccaacaaacttcacaagggaagtgtttatcctacctcaagagtttgagcacatgtcctctgaacccaaggcagcccctgatccacagagaataggagagacagtgccttaggtccagtgggagcaggtacgggctgctatgctggggagtctaggacaacatgagaactagtgcagctgggaggcaggcaggcagtggggctctgcctggcaccgatccacaggtcaccaaaggctttccaagtcctcacttctgccctggaagcagatggctgcgacagccaggtgtgcagaagcccgctgaggcctacaatgagcacatcagcacctcatgatggctctgaccctgagaacagaagcaacccatggccttggggagaagatgctgggaatttttatgatcatttatctacagtcttaaagaatttaaaaaagaagatgagtctctaatcccagcactctgagagtccgaggcaggtggatcacttaagcccaggggttccagaccagcctgtgcaacatggcaaaacccatctctttaaaaaaaaaaaaaaaaaagaaaaaaaaaaagaaaataaacgaggcaaaattagctgggcatggtggtgcaagcctgtagtcctagatactcgggaggctgaggtgggaggatcgcttgaacctggaaggcagaggctgccgtgagctatgactgtgccactacactccagcctgggtgacagaacgagaccctgtctccaataattaaaacaaaaaaaatagaaaagatgatgatgatagtactaaatattgactttattctccaaaatttttaacccaatcttattattcaaagaaagtcattttttaaacttaggtatttttccttttttaaaaaaattaagtttacacatctatgctgtaacctcccctccccgcccacacgtgtacttcacactgtgcaccacccaggctctcaacttgaaggaagtgacccaggacccccaatcagaattgtggccatgtggaaacaaccactctgcagctagaaggctcttacggctctggtggagggatgtccacagtgcagatgtcctcagctcacgactgtgacagcgacttctcggcaagagtgccccccacccccaacccacttgagacaggtgtgttttgtagaagagactgggccatgcacagcacacacccggtcttccagccaaaagcaaatgagtgctggtggagggggcttgcagtggaacacccactctgctttaattgaataaccccaacttaaaatgtatgcactaggctgactttaggcacttttctaaagcaacatggcattatcccaaggaaagtgacagaaaaaaaaactctgccaaggcccacagaagtctgctcccacgaggacctcagcctgctctgctgagggtggctgcgccctcccaagcctcacctggagcttgcgggccatggccaccacctcatggtcaggagggttgtacttatagcagttggagaacatcaatcggacgtcagcaccaaactcctgagcatcacggtactcacgggcctccagtttagactggaaaacaagacaagtccctgttagctgtgtctgcccatgtgaccatggagaagtggctggcagcagacgaaagggatgcctagaagagcaagttggtttcttggctctcagtttggtcaagactctagtggggggacaggccatcaccccagttctcagcctcagggctgggactggccctgccaacatcccagctcacctgcctggctcgcaggcaggtggactcagaacccacaggaggcggagcctgagccagaaatgacggggagaactgatggagagggctgggcccagctcatcgtgatgacacagtacaggagctggtcaaagggacacagcactgttccctgctctgcctgccccatgcttgggactccactcccagaagaagaaacacgtaacaagtcttaaaactaaaaaagcctcaaaacactatggtgaaaatgtcggcacttacagcgtgcctccaacacagaggagacagcaaggccttctccaccacaaagaggctggcaaggccgccaagccccaactcccatggagcatctactgtgtgggcaaggtacctggggcactgggcccccaaggccagagggcttgtcttgcttttcctcagactaggggaaggggcagcacaggcatctgtagccacaatggccaagcaccaggtctcagagcacgtcccacagcagacagcagggggcgctgagtttcttcgagttggcgggaaaaatccacgcagccaccgttccagggcctgggcttcctcttggactaaaaggtctaggaaggttctgatgtggagggacagcctgcccaccttgtcccttccctcaggcacatcccgctaaccttgattgtgctcatgtccatggggtgcttgatgatgtcacagtagtcgtgtaggcccagtgcctccacgtccacaggcttgtagaagggccaggcgtaggcggcgtgcttcttggcaaacatctccttgaggatgccgctgcagcacttgagctgctccgagaccttgctgctcttctctggtgctgggtgctgctgagagtcgggcacgtccttctttggaggtttcacaggccggctgctctcccgccgctggcccagcttggtggtcttgggctccgggggcagcgagggtggctcgtgaatggggtcaatggtggtgggggtggtggtgtctgctttcctcttcactcccttctttgtctgccaagaacacggacgccaacaggcacagtcagaagtggcagccggcacagctgccctcagggtcacccccaaagccaggccctgtcttggggcccatcgctcacacagaatggacccaaagataattgcacaggcaaagggccaaggacaggcagatcgtgtcccaccactctgctctgcagctgggacaggacctgcctgtggcaggagaaagcataaaactgtgtgctcacttggaagccaagggacctctggaggctgcttagtacctaacaccagggcctctccccaagtgcccaggggaggcacaattcagagactatttagatggcccatctgcaaatatgcacagtctaaataaaagaggtgcagggggcaggaacccactaggaggtgtgacacaattatgggacctttgagtcagtggggcggctgtttctgggctgcaatttcaacacagcaagatctccccagaaacacccaccagtgcccgggacccaggacaggctctgtgtaaagcacgggcaaggacagggccgctctcctccctggtgaagcagccctccagagtccaggagactcaccttcacaggctgtggggtggccgcgatgatgggtgggtggctctgtacgggctggggagctggagcgggtgggggttgtggctgggggggcactggcgggggcgtctgcagtggctggggaggcaccactgtcatgacaggggtctggacgatgaggtccggggtgacggcagggaaggggtgaggcgtggcctgcacaggaggaggattcggctgaggggtctgggtctgcggaggagtcgatgcttgagttgtgtttggtaccgtggaaacgccaggttttgctgtccctacaaatcataataagacggcgagttagagaccatgctgacatccacatgctggctgacctcgtggggacatacaccacacacagtgaacgcacattcgcgtgttgcatttgcctgagagacgaacatcccagactccactctgccaaacccttccacctgtccctggggctcaaagaacagcagaaaaccgcacttcccatttcctagggcgtcctggctgcaactcccaactcaggcacaaagaccccttcatgaagcctggaggcccaggctcaaggagcaggaagcacatgctgacaggggacagagagcgggaaaacactcctcagagcttccaggcacagctgcccttgttcccagaagcactgccttctgagtaagagcttctggcagggagccggcagtagccgcccttgccaaggtttctggaaactggcaggcccccacctcctggaaccacagggggcagctggactgctgcccttcatgactggctcgagcgctgcctcccagcagctgtgcaggcagtccgaacacacaatggtcacgtggcacacccaaccagctgcaaaaagtgaggggttatggccagagctctaacaaaactgggtggaccagggtgagaatgtctaggagcataacctgggtcaccacatccttcgtgtgcatgctggctgccatgaaaatggacaaagacacccagtgccttcatctatcagctcaagcagcacccatgttccctgtgcatctcaaggagcaagggtgagattctgctgagtttcccaattctccttcctcaggcacaactcctgggcttctcccagactggaataggagatgccaccacgtctgtctgcccagcgtgcagcccaaacccctggtcccagggagtgaggaccaatatcctcatgtaaggataagggatgtgacttacatgaggatcaggaatgtgatctgataggtacaagagccacagggtgacacgtgagactagttctccttggttacctttgggtgcagcaggggatgtagcaaccccctttctgccaccacttcagaacagcatgggaggccaggtgaaaggctgctgctcagagaagcaaacaactcacactcagggaccacctcgcaaatgtcacccttctgcagcatggagttgcaaaggacaagatgtagccagtagctgctggagagtttcctagacagaatcatttcctagagtgggcagctctgccagctggcccctggaaccctgtgtcagttgggggtggtgacaggcagagatggatgaggagggctttcacttcagatgactgagcatcagaagcagggataactctgacaccagtgcattaaggtaattccaatcacaaacctcaaagtttagaactgagatgttagttaaaatgatttctggtcatgttaatgtccttaaaatggaaaagtaaaaaccacctatgatactggcagggtgaagtgttcaacaaacgtcaaagtgctttgagaagttacactttccttttaccctaagtccatcacctaccctcagaaaaggcagggcccaattatttctgcttagaagaggccacagggccacatgaccctatgggatggatccaacaccaaacccaatctcacatgtccaaacaccaacacggcatgcagtatataatgtcacatcctcctgccactcccagccccccaccaaacttggaaaagggacaaaggtcggggagaaagccaatttacttgctatttcagtactctacctgtttctttcctcccacgtcctcttccttttgcctggactatcatgatctcggtttcttctgtgggtagctcatttattttttgcaagaagagcttttccagagcttctgccattaagactatgtcatctccaggctggataaggagacacaggggtagagtcagagggccctcccctccccacctctgtagacagggcaccccatgccacccaccccctgcccccaaaacatgaagcgtcgtattccgggtccttccccgatctgcacccaaaggagccacaatggctacactaccaggtatcacagaggcttgtgttcccacaggtgtgctgagcctacacttgtctacctgcacctccctcatcttttaagctctggcctatctgagaagccatcctaatgccttaagcatctaccaagctcagccaatcactgaaaggggccctcctttgacacaattcaatttcaccagaaatacgtgaatgacacaggatttcttgccatgatacattagcagggccccagcaatgctccccaacgtacccccatggcagctcaccaggactctgggcactgcacatctcacactagttgaggggtcccactctgtcacctcatctataacatgctgttcccactcagttctaaactgagagcctggtctgcacattttgctcaaacctaagtggtgagagctgagactttaaaaaacacagtaaacttcaagaaactcacctacccatctcttctgggcatttttacccactacaccagcagaggtcaaaagctgagttctgtatagtgtctccctccagagtcgttctatttgaaagggttgattttaatcacatctgtggtctgaactgtaaacaaatgatctggcttcatggcacagccgagccgaaaggtttggctacggggttaggaagtgaccttcaggtctacatgtaggatcggggggacacttcatctatggccaagagttattgacagtcatgtccacagttccagcagaccatcacccacagagcaactgagctctgcgccggccccatcacaccctctctcacgtggaggtctctgtttgggcacacctctgcacaactacattttagcaggctctttcctaacaatttacatcacccaaatggactttgtcatgagtcgccactattctcctcacaccttctctactagacatgctcaggaaccaaaatcccaagggcaccagaagcacatgcttcaggctaacaaggacagcttgcaagttcaacatggccagcaccctcctcccagcaaaaaagcccacacccagtcacaggaacaaccacaggtctcattacctctacctccaagggtctcctcttgccagactccaagcccagccacacccaacacccctgcccacgggctcctgacatgcccactgccgtcgcctcccctcctctctccccagggcagctggacacccacccctacatctcaccttgttgtagatgtaacaatttgtaaacatagtgttgaagtcctggatacattcctgagcattccagtaatagttgttttccaagcgcttctttattgttcccatatccataggcgttttaatgatcttatagtaatcctggagagcagagagcaaaagtccagtgtcacctaggcagccaggcagcacaaacgctgggctggccaggctggcctggggacctcacccctggctgctccacaggtaaatccacgggagccacagctcctactggcatcagcaagtgatcttgagggggaacaccaaaataaagtggagcggggaagggttcctggatgctggggagtaattcagagggaaacgtacaagtttccaaccactctatcttgaaataggctgccttcattgaaatgcacattttttctttttatggatactaaaatattctttcacttttaataaataagggggaaaccaggcagccttttaaaaagcaatcctcacctagtaagacaacccatggcaggtcctacttgtcccatttgaaattttacatgtccataaaatgcaaattcccctggcgccaatgctggagtccacttgtaacccttgaccctggcactcctgcctccttgtcaccatgaagagctgcggagcataagtttccacccctagtcaaattcactggacttaacgtttctttcatttgcacacagtctaccactaaggattctttagtatccagatactgctgactgaccaggttaactgccaaatgacaaatctggacagccttcacaatccttagtttaaaaaaataaaaataaaaaatccaccatcccaaccctcaaactttcagatcttactaagtgccaaacagaaggagccagataacagatgtaaacgtcttaaagtgcccgaaatcaaaccacgttattctcacaacttcttctctaaatctaaacctattctaaaactaagttcatttttaaaaacaaccacagaaggaatgtgcggacagatcaagcagtgcctgggtttccaggtgtgaggatcacagagggccaggcagggggctgcacccagacagcaagtccccaatgactagaccagctcaggctgttggcctgacccaccgatagccaaactttctgagtcatcactctgactgaggacaggtgctcctgtaattaagttcaagtggtgtatcagaaccaaatccaagtggagtcccacggtagaggcacaggctgcaggtggagaggctgttgcagggagaagagagcaggtatactaacagcaagcactgaggccctgctcgggatggtagtggacagcgttggaaaggccagcacagggtatgggtgtggggtttttgagatgttctgacatccacgagaaagagtcaagggccacctgcttacatgtaaggttttggcacagctcatggaactgccctgggggtgagggctacaagtgcacacccaggcaccttaggcaagtggtcaagaggggagactggaagcagactatcggtgccactttctatgctctgacctcctttcccaagagaaccaagtctgaatagatctatcccagtggaaatttaaattttattctatggccaaaggccactgaattggccacagtgtgtcacatgtctcaatggccaacagaatagtgaagaggaattggggtgaaggcagagagaacggtgggggaagaggaactgccagcctggatagtacggtttggggccatgataggagatcaaaatgcgggtgaagacctagaaggcatcagaaaacagacaaggaatactttttagggtggccttattaacatctcctgaagaaggaagtccccagaatacagatttagcatgatgatgctcctgatcaagcatcccctttggctcagcccccaccaaatgccctgagaccactctcactcagcaagctccttcctcagctcctcactctcccatctctgaagacatcagatcagggccaccatggatcagtcccttcttcttatgtcagagctaatgagggcgacgtcaacagtcatagggacaggtagattcacactctgcctggaccaagaacgtcctggccactctccagcctgcacttcagagactgaccttgaactcccaaaccccatggcctaaccaatcacccatacttgtctgtcccgctggccagcactgttccctcaacctcaactgggatgctgctgcttctcggcaatctcaagtcccccagcccttccttctcctgtcttcaccttctcagccctccctgcttcagttcccactgcgttttcttgtaacaagctgacccctctgctattctggcactgatgcgtttcccttcaaagccaagcttcttgaaatggtggcttcacatcctcaccttctcattcacaccgtcgcaatctggtttctacttctgtgtcacgtcttcctaaggtcaccaatgccctcctaaaggtcaacaggatgatcaggtcggaatcctcatctctttgacctccctgtagcttctgatgttggaatctggggaaatgctctcttctgtaggccttctgccttccctccaacccctaagggcctcacaacgctctgggttccactccagtccccagttccttctctcctggttctgtgcatctgctctgagaatttcactctcaactatggaccctgtccagcccagctagagattggtgcagtgagctcccccaacagtctccctcccgcaggccaacacagctgactgcctccactcctcatcttggtcaatggagagcgtggcgcctcttctgcctccatacctttcctgagccatttcaagacccagacacgtactgccctgttctgattatcttcccccgagtgcttgacacgtattcagtgaagcaatagcaccagacagttcttacagacacaatgactttaaacttgggagtaaggatgcctggcagaggctaaggccgctcctacacaccagtggctctggtgtgcacacaccagtggctctggtgtgcacacaccagtctgggtcagtggctgcaaccacctaatgcacttgccagggggtgtacaggacttggcaaacagcaaaggacaatttaaagacaattagtctttggtgctttaaagaaaaaaaaactgaagcatttattccatctgctccctgaggttcagctggagatacaggtttgcttgcagaacagtttgctagggacatgggcatatggaaaactctccagggaaagagaagaggggcgggctcacaaaagcacaagagcaagtgtgctgtagccagagggagggagggcaacaccacctgtgcagctgcagccacagccacctttctccaactggggacgcctccatgtacttgcactccctgcgaggtaatcccactatctttccccaccgtgtgctccgggtgacagtgacgatgatgctaggtgaccactactgcagcaagccaattcccctctagcatagtttgatcaactacgtctacttatccttgatcaactatgtccacacctgagaaatgaaggggctaggatggccaagtgctctgacttcaactcaccatggaactggaagtagatctgtgggccttcctttctcccactgcttcaacctctacacaattttccccaggattccaatatctaagaaaccagttattgttaagccaattagctcattctttctccctgatttcaactgatcagaactgctgaaaccactcactcaagggccctgcagctctcaccatgattccaaatgcatctcctaccctccccccaggaactgccctgaccaggagacatgcaggagacgacctccaggacggccaccctgggccctgcccacactactcacagggaggttcagcttgacggcatccacaggctgctggaaaggccatgcaaactggtgtttccatagtgtcttgagcaccactctgagcaggtattgcagttggttggtctgcctcttgggcttgttagggttggaggtctctgggggcggggggttggtgctggctgcgttggctggctggggttgggcctgggcctgtgttgtagacatttgggaagtttctagtccatcccccattactggcagatttctcaatctcgtcccagggccgctctccgcagacat

SEQ ID NO: 113 - DNA, 20 bp, Artificial
  Synthetic
agcccaaagttagacgcttt

SEQ ID NO: 114 - DNA, 20 bp, Artificial
  Synthetic
aggtaggctgaggcagaagg

SEQ ID NO: 115 - DNA, 20 bp, Artificial
  Synthetic
tgcctcagcctacctttttc

SEQ ID NO: 116 - DNA, 20 bp, Artificial
  Synthetic
ccttcttgtctcagccttcc

SEQ ID NO: 117 - DNA, 20 bp, Artificial
  Synthetic
atgctgggagctgacttacg

SEQ ID NO: 118 - DNA, 20 bp, Artificial
  Synthetic
agggaaggaaccttgcagat

SEQ ID NO: 119 - DNA, 20 bp, Artificial
  Synthetic
gctcagtggtagagcgcttg

SEQ ID NO: 120 - DNA, 19 bp, Artificial
  Synthetic
ctcacctgagacgctaggc

SEQ ID NO: 121 - DNA, 20 bp, Artificial
  Synthetic
ggctgtttgttctgctctcc

SEQ ID NO: 122 - DNA, 20 bp, Artificial
  Synthetic
cctcctcctcctcctcactt

SEQ ID NO: 123 - DNA, 18 bp, Artificial
  Synthetic
cggagcctggtgcttctc

SEQ ID NO: 124 - DNA, 20 bp, Artificial
  Synthetic
gagtacccagctgacggaag

SEQ ID NO: 125 - DNA, 20 bp, Artificial
  Synthetic
gcagttgggagctgaggtag

SEQ ID NO: 126 - DNA, 20 bp, Artificial
  Synthetic
ctctggccacactgaaacaa

SEQ ID NO: 127 - DNA, 20 bp, Artificial
  Synthetic
tcttggttcagcaggtctca

SEQ ID NO: 128 - DNA, 20 bp, Artificial
  Synthetic
ggtgtgatgacacaaaccac

SEQ ID NO: 129 - DNA, 20 bp, Artificial
  Synthetic
gccaagactggctttgatct

SEQ ID NO: 130 - DNA, 20 bp, Artificial
  Synthetic
tgcctgttctgtaccctcaa

SEQ ID NO: 131 - DNA, 20 bp, Artificial
  Synthetic
gagagggtgggggtgattat

SEQ ID NO: 132 - DNA, 20 bp, Artificial
  Synthetic
gctgtggacaatctgaagca

SEQ ID NO: 133 - DNA, 20 bp, Artificial
  Synthetic
taccagtggagcccaatctt

SEQ ID NO: 134 - DNA, 20 bp, Artificial
  Synthetic
ccctgtccagatggctactc

SEQ ID NO: 135 - DNA, 20 bp, Artificial
  Synthetic
acgtctttggctgtggagtt

SEQ ID NO: 136 - DNA, 20 bp, Artificial
  Synthetic
acacccaatcctatgcacaa

SEQ ID NO: 137 - DNA, 20 bp, Artificial
  Synthetic
ggccataaaatccagtgtcc

SEQ ID NO: 138 - DNA, 20 bp, Artificial
  Synthetic
ctgtccccgttcagctctaa

SEQ ID NO: 139 - DNA, 20 bp, Artificial
  Synthetic
ctccatgtattggagcatgg

SEQ ID NO: 140 - DNA, 20 bp, Artificial
  Synthetic
catgggacttcctaggagca

SEQ ID NO: 141 - DNA, 21 bp, Artificial
  Synthetic
cctgaagtgttccagatggtc

SEQ ID NO: 142 - DNA, 19 bp, Artificial
  Synthetic
gtctctggtggcagcaatc

SEQ ID NO: 143 - DNA, 20 bp, Artificial
  Synthetic
gggcttgtcctgagtattgg

SEQ ID NO: 144 - DNA, 21 bp, Artificial
  Synthetic
cccagaacgttgttggattag

SEQ ID NO: 145 - DNA, 20 bp, Artificial
  Synthetic
ggagtgatggcctgttgttc

SEQ ID NO: 146 - DNA, 21 bp, Artificial
  Synthetic
agaaccagccactcacattta

SEQ ID NO: 147 - DNA, 20 bp, Artificial
  Synthetic
ggtcttgctcatggcctaac

SEQ ID NO: 148 - DNA, 20 bp, Artificial
  Synthetic
aagaggaaatgccacaagga

SEQ ID NO: 149 - DNA, 22 bp, Artificial
  Synthetic
ggaagggattgattgtagacct

SEQ ID NO: 150 - DNA, 20 bp, Artificial
  Synthetic
agggggaaggaacagctaag

SEQ ID NO: 151 - DNA, 21 bp, Artificial
  Synthetic
tgaagtttttgtcagggaacc

SEQ ID NO: 152 - DNA, 23 bp, Artificial
  Synthetic
cgcatagaattcataacttcctc

SEQ ID NO: 153 - DNA, 20 bp, Artificial
  Synthetic
ctgggttggtagttgggaat

SEQ ID NO: 154 - DNA, 20 bp, Artificial
  Synthetic
caacacctgcagtcctcaag

SEQ ID NO: 155 - DNA, 20 bp, Artificial
  Synthetic
gcccagtctgcaattcttct

SEQ ID NO: 156 - DNA, 20 bp, Artificial
  Synthetic
gatcaggctttgcacacaga

SEQ ID NO: 157 - DNA, 20 bp, Artificial
  Synthetic
ttgtcctaaatgccccatgt

SEQ ID NO: 158 - DNA, 18 bp, Artificial
  Synthetic
cctgggcagtgatgaagg

SEQ ID NO: 159 - DNA, 19 bp, Artificial
  Synthetic
ctccatgccacagcagact

SEQ ID NO: 160 - DNA, 21 bp, Artificial
  Synthetic
tcagcttgccaagagagtaaa

SEQ ID NO: 161 - DNA, 20 bp, Artificial
  Synthetic
agacagaaacgccaatccag

SEQ ID NO: 162 - DNA, 20 bp, Artificial
  Synthetic
caagtgaactggtcgtggtg

SEQ ID NO: 163 - DNA, 18 bp, Artificial
  Synthetic
cagcagctccagccacag

SEQ ID NO: 164 - DNA, 22 bp, Artificial
  Synthetic
tgcttgtgaacaagacaaacag

SEQ ID NO: 165 - DNA, 20 bp, Artificial
  Synthetic
agcttgtttggaccacatga

SEQ ID NO: 166 - DNA, 19 bp, Artificial
  Synthetic
aggcagggaggacactcac

SEQ ID NO: 167 - DNA, 20 bp, Artificial
  Synthetic
cagcccctggtggtagtaaa

SEQ ID NO: 168 - DNA, 20 bp, Artificial
  Synthetic
acttgaggacttggctgtgg

SEQ ID NO: 169 - DNA, 20 bp, Artificial
  Synthetic
tcacctgcctcttgaccttt

SEQ ID NO: 170 - DNA, 19 bp, Artificial
  Synthetic
ccaactccctctgctggtc

SEQ ID NO: 171 - DNA, 20 bp, Artificial
  Synthetic
gagccgagaggatgaagatg

SEQ ID NO: 172 - DNA, 20 bp, Artificial
  Synthetic
gctgcccctaacactatgga

SEQ ID NO: 173 - DNA, 21 bp, Artificial
  Synthetic
tggcagctacaattgacatga

SEQ ID NO: 174 - DNA, 19 bp, Artificial
  Synthetic
ctgctccagtccacacagg

SEQ ID NO: 175 - DNA, 20 bp, Artificial
  Synthetic
acgtttgtgacgtcctaccc

SEQ ID NO: 176 - DNA, 20 bp, Artificial
  Synthetic
gccacagtcacacactaccc

SEQ ID NO: 177 - DNA, 22 bp, Artificial
  Synthetic
ctcttctcctcagacacagtgg

SEQ ID NO: 178 - DNA, 20 bp, Artificial
  Synthetic
ggggctccaatttaaaaaca

SEQ ID NO: 179 - DNA, 20 bp, Artificial
  Synthetic
gaaagggagagcctgaggag

SEQ ID NO: 180 - DNA, 18 bp, Artificial
  Synthetic
ccaggccagggagttaca